SEQUENCE LISTING (734 sequences)

SEQ ID NO: 1 - protein, 9 aa, Artificial sequence
  synthetic
IMGFIGFFV

SEQ ID NO: 2 - protein, 9 aa, Artificial sequence
  synthetic
MGFIGFFVK

SEQ ID NO: 3 - protein, 9 aa, Artificial sequence
  synthetic
GFIGFFVKL

SEQ ID NO: 4 - protein, 9 aa, Artificial sequence
  synthetic
FIGFFVKLI

SEQ ID NO: 5 - protein, 9 aa, Artificial sequence
  synthetic
IGFFVKLIH

SEQ ID NO: 6 - protein, 9 aa, Artificial sequence
  synthetic
GFFVKLIHI

SEQ ID NO: 7 - protein, 9 aa, Artificial sequence
  synthetic
FFVKLIHIP

SEQ ID NO: 8 - protein, 9 aa, Artificial sequence
  synthetic
FVKLIHIPI

SEQ ID NO: 9 - protein, 9 aa, Artificial sequence
  synthetic
VKLIHIPIN

SEQ ID NO: 10 - protein, 9 aa, Artificial sequence
  synthetic
KLIHIPINN

SEQ ID NO: 11 - protein, 9 aa, Artificial sequence
  synthetic
LIHIPINNI

SEQ ID NO: 12 - protein, 9 aa, Artificial sequence
  synthetic
IHIPINNII

SEQ ID NO: 13 - protein, 5 aa, Artificial sequence
  synthetic
FIGFF

SEQ ID NO: 14 - protein, 5 aa, Artificial sequence
  synthetic
IGFFV

SEQ ID NO: 15 - protein, 5 aa, Artificial sequence
  synthetic
GFFVK

SEQ ID NO: 16 - protein, 5 aa, Artificial sequence
  synthetic
FFVKL

SEQ ID NO: 17 - protein, 5 aa, Artificial sequence
  synthetic
FVKLI

SEQ ID NO: 18 - protein, 5 aa, Artificial sequence [sequence omitted]

SEQ ID NO: 19 - protein, 5 aa, Artificial sequence
  synthetic
KLIHI

SEQ ID NO: 20 - protein, 5 aa, Artificial sequence
  synthetic
LIHIP

SEQ ID NO: 21 - protein, 5 aa, Artificial sequence
  synthetic
IHIPI

SEQ ID NO: 22 - protein, 5 aa, Artificial sequence
  synthetic
HIPIN

SEQ ID NO: 23 - protein, 5 aa, Artificial sequence
  synthetic
IPINN

SEQ ID NO: 24 - protein, 5 aa, Artificial sequence
  synthetic
PINNI

SEQ ID NO: 25 - protein, 15 aa, Artificial sequence
  synthetic
IGFAIMGFIGFFVKL

SEQ ID NO: 26 - protein, 15 aa, Artificial sequence
  synthetic
GFAIMGFIGFFVKLI

SEQ ID NO: 27 - protein, 15 aa, Artificial sequence
  synthetic
FAIMGFIGFFVKLIH

SEQ ID NO: 28 - protein, 15 aa, Artificial sequence
  synthetic
AIMGFIGFFVKLIHI

SEQ ID NO: 29 - protein, 15 aa, Artificial sequence
  synthetic
IMGFIGFFVKLIHIP

SEQ ID NO: 30 - protein, 15 aa, Artificial sequence
  synthetic
MGFIGFFVKLIHIPI

SEQ ID NO: 31 - protein, 15 aa, Artificial sequence
  synthetic
GFIGFFVKLIHIPIN

SEQ ID NO: 32 - protein, 15 aa, Artificial sequence
  synthetic
FIGFFVKLIHIPINN

SEQ ID NO: 33 - protein, 15 aa, Artificial sequence
  synthetic
IGFFVKLIHIPINNI

SEQ ID NO: 34 - protein, 15 aa, Artificial sequence
  synthetic
GFFVKLIHIPINNII

SEQ ID NO: 35 - protein, 15 aa, Artificial sequence
  synthetic
FFVKLIHIPINNIIV

SEQ ID NO: 36 - protein, 15 aa, Artificial sequence
  synthetic
FVKLIHIPINNIIVG

SEQ ID NO: 37 - protein, 5 aa, Artificial sequence
  synthetic
IMFGF

SEQ ID NO: 38 - protein, 5 aa, Artificial sequence
  synthetic
MGIFF

SEQ ID NO: 39 - protein, 5 aa, Artificial sequence
  synthetic
GFGFV

SEQ ID NO: 40 - protein, 5 aa, Artificial sequence
  synthetic
FIFVK

SEQ ID NO: 41 - protein, 5 aa, Artificial sequence
  synthetic
IGFKL

SEQ ID NO: 42 - protein, 5 aa, Artificial sequence
  synthetic
GFVLI

SEQ ID NO: 43 - protein, 5 aa, Artificial sequence
  synthetic
FFKIH

SEQ ID NO: 44 - protein, 5 aa, Artificial sequence
  synthetic
FVLHI

SEQ ID NO: 45 - protein, 5 aa, Artificial sequence
  synthetic
VKIIP

SEQ ID NO: 46 - protein, 5 aa, Artificial sequence
  synthetic
KLHPI

SEQ ID NO: 47 - protein, 5 aa, Artificial sequence
  synthetic
LIIIN

SEQ ID NO: 48 - protein, 5 aa, Artificial sequence
  synthetic
IHPNN

SEQ ID NO: 49 - protein, 9 aa, Artificial sequence
  synthetic
SGRFVKLIK

SEQ ID NO: 50 - protein, 9 aa, Artificial sequence
  synthetic
TTGFVKLIR

SEQ ID NO: 51 - protein, 9 aa, Artificial sequence
  synthetic
DAEFVKLIK

SEQ ID NO: 52 - protein, 9 aa, Artificial sequence
  synthetic
GSNFVKLIR

SEQ ID NO: 53 - protein, 9 aa, Artificial sequence
  synthetic
SSNGFFVKR

SEQ ID NO: 54 - protein, 9 aa, Artificial sequence
  synthetic
RIEGFFVKR

SEQ ID NO: 55 - protein, 9 aa, Artificial sequence
  synthetic
SGYGFFVKR

SEQ ID NO: 56 - protein, 9 aa, Artificial sequence
  synthetic
KSAGFFVKP

SEQ ID NO: 57 - protein, 9 aa, Artificial sequence
  synthetic
SSVKLIHIP

SEQ ID NO: 58 - protein, 9 aa, Artificial sequence
  synthetic
EITKLIHIK

SEQ ID NO: 59 - protein, 9 aa, Artificial sequence
  synthetic
ESPKLIHIK

SEQ ID NO: 60 - protein, 9 aa, Artificial sequence
  synthetic
AIQKLIHIR

SEQ ID NO: 61 - protein, 9 aa, Artificial sequence
  synthetic
RQRFFVKLF

SEQ ID NO: 62 - protein, 9 aa, Artificial sequence
  synthetic
RKYFFVKLI

SEQ ID NO: 63 - protein, 9 aa, Artificial sequence
  synthetic
KGQFFVKLF

SEQ ID NO: 64 - protein, 9 aa, Artificial sequence
  synthetic
RTQFFVKLI

SEQ ID NO: 65 - protein, 9 aa, Artificial sequence
  synthetic
KNAGFFVKF

SEQ ID NO: 66 - protein, 9 aa, Artificial sequence
  synthetic
KGAGFFVKI

SEQ ID NO: 67 - protein, 9 aa, Artificial sequence
  synthetic
KARGFFVKW

SEQ ID NO: 68 - protein, 9 aa, Artificial sequence
  synthetic
EAQGFFVKF

SEQ ID NO: 69 - protein, 9 aa, Artificial sequence
  synthetic
RAMVKLIHL

SEQ ID NO: 70 - protein, 9 aa, Artificial sequence
  synthetic
KGFVKLIHF

SEQ ID NO: 71 - protein, 9 aa, Artificial sequence
  synthetic
RMMVKLIHL

SEQ ID NO: 72 - protein, 9 aa, Artificial sequence
  synthetic
RPMVKLIHF

SEQ ID NO: 73 - protein, 5 aa, Artificial sequence
  synthetic
FVKLI

SEQ ID NO: 74 - protein, 5 aa, Artificial sequence
  synthetic
FVKLI

SEQ ID NO: 75 - protein, 5 aa, Artificial sequence
  synthetic
FVKLI

SEQ ID NO: 76 - protein, 5 aa, Artificial sequence
  synthetic
FVKLI

SEQ ID NO: 77 - protein, 5 aa, Artificial sequence
  synthetic
GFFVK

SEQ ID NO: 78 - protein, 5 aa, Artificial sequence
  synthetic
GFFVK

SEQ ID NO: 79 - protein, 5 aa, Artificial sequence
  synthetic
GFFVK

SEQ ID NO: 80 - protein, 5 aa, Artificial sequence
  synthetic
GFFVK

SEQ ID NO: 81 - protein, 5 aa, Artificial sequence
  synthetic
KLIHI

SEQ ID NO: 82 - protein, 5 aa, Artificial sequence
  synthetic
KLIHI

SEQ ID NO: 83 - protein, 5 aa, Artificial sequence
  synthetic
KLIHI

SEQ ID NO: 84 - protein, 5 aa, Artificial sequence
  synthetic
KLIHI

SEQ ID NO: 85 - protein, 5 aa, Artificial sequence
  synthetic
FFVKL

SEQ ID NO: 86 - protein, 5 aa, Artificial sequence
  synthetic
FFVKL

SEQ ID NO: 87 - protein, 5 aa, Artificial sequence
  synthetic
FFVKL

SEQ ID NO: 88 - protein, 5 aa, Artificial sequence
  synthetic
FFVKL

SEQ ID NO: 89 - protein, 5 aa, Artificial sequence
  synthetic
GFFVK

SEQ ID NO: 90 - protein, 5 aa, Artificial sequence
  synthetic
GFFVK

SEQ ID NO: 91 - protein, 5 aa, Artificial sequence
  synthetic
GFFVK

SEQ ID NO: 92 - protein, 5 aa, Artificial sequence
  synthetic
GFFVK

SEQ ID NO: 93 - protein, 5 aa, Artificial sequence [sequence omitted]

SEQ ID NO: 94 - protein, 5 aa, Artificial sequence [sequence omitted]

SEQ ID NO: 95 - protein, 5 aa, Artificial sequence [sequence omitted]

SEQ ID NO: 96 - protein, 5 aa, Artificial sequence [sequence omitted]

SEQ ID NO: 97 - protein, 9 aa, Artificial sequence
  synthetic
HCWYFEGLY

SEQ ID NO: 98 - protein, 9 aa, Artificial sequence
  synthetic
CWYFEGLYP

SEQ ID NO: 99 - protein, 9 aa, Artificial sequence
  synthetic
WYFEGLYPT

SEQ ID NO: 100 - protein, 9 aa, Artificial sequence
  synthetic
YFEGLYPTY

SEQ ID NO: 101 - protein, 9 aa, Artificial sequence
  synthetic
FWFLLMMGV

SEQ ID NO: 102 - protein, 9 aa, Artificial sequence
  synthetic
WFLLMMGVL

SEQ ID NO: 103 - protein, 9 aa, Artificial sequence
  synthetic
FLLMMGVLF

SEQ ID NO: 104 - protein, 9 aa, Artificial sequence
  synthetic
LLMMGVLFC

SEQ ID NO: 105 - protein, 9 aa, Artificial sequence
  synthetic
LMMGVLFCC

SEQ ID NO: 106 - protein, 9 aa, Artificial sequence
  synthetic
MMGVLFCCG

SEQ ID NO: 107 - protein, 9 aa, Artificial sequence
  synthetic
MGVLFCCGA

SEQ ID NO: 108 - protein, 9 aa, Artificial sequence
  synthetic
GVLFCCGAG

SEQ ID NO: 109 - protein, 9 aa, Artificial sequence
  synthetic
VLFCCGAGF

SEQ ID NO: 110 - protein, 9 aa, Artificial sequence
  synthetic
LFCCGAGFF

SEQ ID NO: 111 - protein, 9 aa, Artificial sequence
  synthetic
FCCGAGFFI

SEQ ID NO: 112 - protein, 9 aa, Artificial sequence
  synthetic
HCWYFEGLY

SEQ ID NO: 113 - protein, 9 aa, Artificial sequence
  synthetic
CWYFEGLYP

SEQ ID NO: 114 - protein, 9 aa, Artificial sequence
  synthetic
WYFEGLYPT

SEQ ID NO: 115 - protein, 9 aa, Artificial sequence
  synthetic
YFEGLYPTY

SEQ ID NO: 116 - protein, 9 aa, Artificial sequence
  synthetic
FWFLLMMGV

SEQ ID NO: 117 - protein, 9 aa, Artificial sequence
  synthetic
WFLLMMGVL

SEQ ID NO: 118 - protein, 9 aa, Artificial sequence
  synthetic
FLLMMGVLF

SEQ ID NO: 119 - protein, 9 aa, Artificial sequence
  synthetic
LLMMGVLFC

SEQ ID NO: 120 - protein, 9 aa, Artificial sequence
  synthetic
LMMGVLFCC

SEQ ID NO: 121 - protein, 9 aa, Artificial sequence
  synthetic
MMGVLFCCG

SEQ ID NO: 122 - protein, 9 aa, Artificial sequence
  synthetic
MGVLFCCGA

SEQ ID NO: 123 - protein, 9 aa, Artificial sequence
  synthetic
GVLFCCGAG

SEQ ID NO: 124 - protein, 9 aa, Artificial sequence
  synthetic
VLFCCGAGF

SEQ ID NO: 125 - protein, 9 aa, Artificial sequence
  synthetic
LFCCGAGFF

SEQ ID NO: 126 - protein, 9 aa, Artificial sequence
  synthetic
FCCGAGFFI

SEQ ID NO: 127 - protein, 5 aa, Artificial sequence
  synthetic
YFEGL

SEQ ID NO: 128 - protein, 5 aa, Artificial sequence
  synthetic
FEGLY

SEQ ID NO: 129 - protein, 5 aa, Artificial sequence
  synthetic
EGLYP

SEQ ID NO: 130 - protein, 5 aa, Artificial sequence
  synthetic
GLYPT

SEQ ID NO: 131 - protein, 5 aa, Artificial sequence
  synthetic
LLMMG

SEQ ID NO: 132 - protein, 5 aa, Artificial sequence
  synthetic
LMMGV

SEQ ID NO: 133 - protein, 5 aa, Artificial sequence
  synthetic
MMGVL

SEQ ID NO: 134 - protein, 5 aa, Artificial sequence
  synthetic
MGVLF

SEQ ID NO: 135 - protein, 5 aa, Artificial sequence
  synthetic
GVLFC

SEQ ID NO: 136 - protein, 5 aa, Artificial sequence
  synthetic
VLFCC

SEQ ID NO: 137 - protein, 5 aa, Artificial sequence
  synthetic
LFCCG

SEQ ID NO: 138 - protein, 5 aa, Artificial sequence
  synthetic
FCCGA

SEQ ID NO: 139 - protein, 5 aa, Artificial sequence
  synthetic
CCGAG

SEQ ID NO: 140 - protein, 5 aa, Artificial sequence
  synthetic
CGAGF

SEQ ID NO: 141 - protein, 5 aa, Artificial sequence
  synthetic
GAGFF

SEQ ID NO: 142 - protein, 5 aa, Artificial sequence
  synthetic
YFEGL

SEQ ID NO: 143 - protein, 5 aa, Artificial sequence
  synthetic
FEGLY

SEQ ID NO: 144 - protein, 5 aa, Artificial sequence
  synthetic
EGLYP

SEQ ID NO: 145 - protein, 5 aa, Artificial sequence
  synthetic
GLYPT

SEQ ID NO: 146 - protein, 5 aa, Artificial sequence
  synthetic
LLMMG

SEQ ID NO: 147 - protein, 5 aa, Artificial sequence
  synthetic
LMMGV

SEQ ID NO: 148 - protein, 5 aa, Artificial sequence
  synthetic
MMGVL

SEQ ID NO: 149 - protein, 5 aa, Artificial sequence
  synthetic
MGVLF

SEQ ID NO: 150 - protein, 5 aa, Artificial sequence
  synthetic
GVLFC

SEQ ID NO: 151 - protein, 5 aa, Artificial sequence
  synthetic
VLFCC

SEQ ID NO: 152 - protein, 5 aa, Artificial sequence
  synthetic
LFCCG

SEQ ID NO: 153 - protein, 5 aa, Artificial sequence
  synthetic
FCCGA

SEQ ID NO: 154 - protein, 5 aa, Artificial sequence
  synthetic
CCGAG

SEQ ID NO: 155 - protein, 5 aa, Artificial sequence
  synthetic
CGAGF

SEQ ID NO: 156 - protein, 5 aa, Artificial sequence
  synthetic
GAGFF

SEQ ID NO: 157 - protein, 15 aa, Artificial sequence
  synthetic
KKHCWYFEGLYPTYY

SEQ ID NO: 158 - protein, 15 aa, Artificial sequence
  synthetic
KHCWYFEGLYPTYYI

SEQ ID NO: 159 - protein, 15 aa, Artificial sequence
  synthetic
HCWYFEGLYPTYYIC

SEQ ID NO: 160 - protein, 15 aa, Artificial sequence
  synthetic
RALSIQRLWYFWFLL

SEQ ID NO: 161 - protein, 15 aa, Artificial sequence
  synthetic
ALSIQRLWYFWFLLM

SEQ ID NO: 162 - protein, 15 aa, Artificial sequence
  synthetic
LSIQRLWYFWFLLMM

SEQ ID NO: 163 - protein, 15 aa, Artificial sequence
  synthetic
SIQRLWYFWFLLMMG

SEQ ID NO: 164 - protein, 15 aa, Artificial sequence
  synthetic
IQRLWYFWFLLMMGV

SEQ ID NO: 165 - protein, 15 aa, Artificial sequence
  synthetic
QRLWYFWFLLMMGVL

SEQ ID NO: 166 - protein, 15 aa, Artificial sequence
  synthetic
RLWYFWFLLMMGVLF

SEQ ID NO: 167 - protein, 15 aa, Artificial sequence
  synthetic
LWYFWFLLMMGVLFC

SEQ ID NO: 168 - protein, 15 aa, Artificial sequence
  synthetic
WYFWFLLMMGVLFCC

SEQ ID NO: 169 - protein, 15 aa, Artificial sequence
  synthetic
YFWFLLMMGVLFCCG

SEQ ID NO: 170 - protein, 5 aa, Artificial sequence
  synthetic
WYELY

SEQ ID NO: 171 - protein, 5 aa, Artificial sequence
  synthetic
YFGYP

SEQ ID NO: 172 - protein, 5 aa, Artificial sequence
  synthetic
FELPT

SEQ ID NO: 173 - protein, 5 aa, Artificial sequence
  synthetic
IQLYF

SEQ ID NO: 174 - protein, 5 aa, Artificial sequence
  synthetic
QRWFW

SEQ ID NO: 175 - protein, 5 aa, Artificial sequence
  synthetic
RLYWF

SEQ ID NO: 176 - protein, 5 aa, Artificial sequence
  synthetic
LWFFL

SEQ ID NO: 177 - protein, 5 aa, Artificial sequence
  synthetic
WYWLL

SEQ ID NO: 178 - protein, 5 aa, Artificial sequence
  synthetic
YFFLM

SEQ ID NO: 179 - protein, 5 aa, Artificial sequence
  synthetic
FWLMM

SEQ ID NO: 180 - protein, 5 aa, Artificial sequence
  synthetic
WFLMG

SEQ ID NO: 181 - protein, 5 aa, Artificial sequence
  synthetic
FLMGV

SEQ ID NO: 182 - protein, 5 aa, Artificial sequence
  synthetic
LLMVL

SEQ ID NO: 183 - protein, 9 aa, Artificial sequence
  synthetic
GGGFEGLYK

SEQ ID NO: 184 - protein, 9 aa, Artificial sequence
  synthetic
ESHFEGLYK

SEQ ID NO: 185 - protein, 9 aa, Artificial sequence
  synthetic
KQWFEGLYR

SEQ ID NO: 186 - protein, 9 aa, Artificial sequence
  synthetic
KNLFEGLYR

SEQ ID NO: 187 - protein, 9 aa, Artificial sequence
  synthetic
RNMMMGVLR

SEQ ID NO: 188 - protein, 9 aa, Artificial sequence
  synthetic
DAKMMGVLK

SEQ ID NO: 189 - protein, 9 aa, Artificial sequence
  synthetic
RQPMMGVLK

SEQ ID NO: 190 - protein, 9 aa, Artificial sequence
  synthetic
NGSMMGVLK

SEQ ID NO: 191 - protein, 9 aa, Artificial sequence
  synthetic
RVVYFEGLR

SEQ ID NO: 192 - protein, 9 aa, Artificial sequence
  synthetic
GAEYFEGLK

SEQ ID NO: 193 - protein, 9 aa, Artificial sequence
  synthetic
GDMYFEGLK

SEQ ID NO: 194 - protein, 9 aa, Artificial sequence
  synthetic
SNLYFEGLR

SEQ ID NO: 195 - protein, 9 aa, Artificial sequence
  synthetic
PIRYFEGLK

SEQ ID NO: 196 - protein, 9 aa, Artificial sequence
  synthetic
AGQYFEGLK

SEQ ID NO: 197 - protein, 9 aa, Artificial sequence
  synthetic
KVRFCCGAA

SEQ ID NO: 198 - protein, 9 aa, Artificial sequence
  synthetic
RGRFCCGAS

SEQ ID NO: 199 - protein, 9 aa, Artificial sequence
  synthetic
KVKFCCGAG

SEQ ID NO: 200 - protein, 9 aa, Artificial sequence
  synthetic
KGPYFEGLS

SEQ ID NO: 201 - protein, 9 aa, Artificial sequence
  synthetic
RDKYFEGLG

SEQ ID NO: 202 - protein, 9 aa, Artificial sequence
  synthetic
TNRYFEGLG

SEQ ID NO: 203 - protein, 5 aa, Artificial sequence
  synthetic
FEGLY

SEQ ID NO: 204 - protein, 5 aa, Artificial sequence
  synthetic
MMGVL

SEQ ID NO: 205 - protein, 5 aa, Artificial sequence
  synthetic
YFEGL

SEQ ID NO: 206 - protein, 9 aa, Artificial sequence
  synthetic
AYTGWTGIA

SEQ ID NO: 207 - protein, 9 aa, Artificial sequence
  synthetic
YTGWTGIAL

SEQ ID NO: 208 - protein, 9 aa, Artificial sequence
  synthetic
TGWTGIALL

SEQ ID NO: 209 - protein, 9 aa, Artificial sequence
  synthetic
GWTGIALLY

SEQ ID NO: 210 - protein, 9 aa, Artificial sequence
  synthetic
WTGIALLYL

SEQ ID NO: 211 - protein, 9 aa, Artificial sequence
  synthetic
TGIALLYLQ

SEQ ID NO: 212 - protein, 9 aa, Artificial sequence
  synthetic
GIALLYLQL

SEQ ID NO: 213 - protein, 9 aa, Artificial sequence
  synthetic
IALLYLQLY

SEQ ID NO: 214 - protein, 9 aa, Artificial sequence
  synthetic
ALLYLQLYR

SEQ ID NO: 215 - protein, 9 aa, Artificial sequence
  synthetic
LLYLQLYRV

SEQ ID NO: 216 - protein, 9 aa, Artificial sequence
  synthetic
DLPDELLYG

SEQ ID NO: 217 - protein, 9 aa, Artificial sequence
  synthetic
LPDELLYGR

SEQ ID NO: 218 - protein, 9 aa, Artificial sequence
  synthetic
PDELLYGRA

SEQ ID NO: 219 - protein, 9 aa, Artificial sequence
  synthetic
DELLYGRAG

SEQ ID NO: 220 - protein, 9 aa, Artificial sequence
  synthetic
ELLYGRAGY

SEQ ID NO: 221 - protein, 9 aa, Artificial sequence
  synthetic
LLYGRAGYL

SEQ ID NO: 222 - protein, 9 aa, Artificial sequence
  synthetic
ALLYLNTEI

SEQ ID NO: 223 - protein, 9 aa, Artificial sequence
  synthetic
LLYLNTEIG

SEQ ID NO: 224 - protein, 9 aa, Artificial sequence
  synthetic
LYLNTEIGP

SEQ ID NO: 225 - protein, 9 aa, Artificial sequence
  synthetic
YLNTEIGPG

SEQ ID NO: 226 - protein, 9 aa, Artificial sequence
  synthetic
HGMAGIYYM

SEQ ID NO: 227 - protein, 9 aa, Artificial sequence
  synthetic
GMAGIYYML

SEQ ID NO: 228 - protein, 9 aa, Artificial sequence
  synthetic
MAGIYYMLM

SEQ ID NO: 229 - protein, 9 aa, Artificial sequence
  synthetic
AGIYYMLMQ

SEQ ID NO: 230 - protein, 9 aa, Artificial sequence
  synthetic
GIYYMLMQP

SEQ ID NO: 231 - protein, 9 aa, Artificial sequence
  synthetic
IYYMLMQPA

SEQ ID NO: 232 - protein, 9 aa, Artificial sequence
  synthetic
YYMLMQPAA

SEQ ID NO: 233 - protein, 9 aa, Artificial sequence
  synthetic
YMLMQPAAK

SEQ ID NO: 234 - protein, 9 aa, Artificial sequence
  synthetic
MLMQPAAKV

SEQ ID NO: 235 - protein, 9 aa, Artificial sequence
  synthetic
RLVHWCHGA

SEQ ID NO: 236 - protein, 9 aa, Artificial sequence
  synthetic
LVHWCHGAP

SEQ ID NO: 237 - protein, 9 aa, Artificial sequence
  synthetic
VHWCHGAPG

SEQ ID NO: 238 - protein, 9 aa, Artificial sequence
  synthetic
HWCHGAPGV

SEQ ID NO: 239 - protein, 9 aa, Artificial sequence
  synthetic
PGVIHMLMQ

SEQ ID NO: 240 - protein, 9 aa, Artificial sequence
  synthetic
GVIHMLMQA

SEQ ID NO: 241 - protein, 9 aa, Artificial sequence
  synthetic
VIHMLMQAY

SEQ ID NO: 242 - protein, 9 aa, Artificial sequence
  synthetic
IHMLMQAYK

SEQ ID NO: 243 - protein, 9 aa, Artificial sequence
  synthetic
RIPDRPYSL

SEQ ID NO: 244 - protein, 9 aa, Artificial sequence
  synthetic
IPDRPYSLF

SEQ ID NO: 245 - protein, 9 aa, Artificial sequence
  synthetic
PDRPYSLFE

SEQ ID NO: 246 - protein, 9 aa, Artificial sequence
  synthetic
DRPYSLFEG

SEQ ID NO: 247 - protein, 9 aa, Artificial sequence
  synthetic
RPYSLFEGM

SEQ ID NO: 248 - protein, 9 aa, Artificial sequence
  synthetic
PYSLFEGMA

SEQ ID NO: 249 - protein, 9 aa, Artificial sequence
  synthetic
YSLFEGMAG

SEQ ID NO: 250 - protein, 9 aa, Artificial sequence
  synthetic
SLFEGMAGA

SEQ ID NO: 251 - protein, 9 aa, Artificial sequence
  synthetic
LFEGMAGAI

SEQ ID NO: 252 - protein, 9 aa, Artificial sequence
  synthetic
FEGMAGAIH

SEQ ID NO: 253 - protein, 9 aa, Artificial sequence
  synthetic
EGMAGAIHF

SEQ ID NO: 254 - protein, 9 aa, Artificial sequence
  synthetic
GMAGAIHFL

SEQ ID NO: 255 - protein, 9 aa, Artificial sequence
  synthetic
MAGAIHFLS

SEQ ID NO: 256 - protein, 9 aa, Artificial sequence
  synthetic
AGAIHFLSD

SEQ ID NO: 257 - protein, 5 aa, Artificial sequence
  synthetic
GWTGI

SEQ ID NO: 258 - protein, 5 aa, Artificial sequence
  synthetic
WTGIA

SEQ ID NO: 259 - protein, 5 aa, Artificial sequence
  synthetic
TGIAL

SEQ ID NO: 260 - protein, 5 aa, Artificial sequence
  synthetic
GIALL

SEQ ID NO: 261 - protein, 5 aa, Artificial sequence
  synthetic
IALLY

SEQ ID NO: 262 - protein, 5 aa, Artificial sequence
  synthetic
ALLYL

SEQ ID NO: 263 - protein, 5 aa, Artificial sequence
  synthetic
LLYLQ

SEQ ID NO: 264 - protein, 5 aa, Artificial sequence
  synthetic
LYLQL

SEQ ID NO: 265 - protein, 5 aa, Artificial sequence
  synthetic
YLQLY

SEQ ID NO: 266 - protein, 5 aa, Artificial sequence
  synthetic
LQLYR

SEQ ID NO: 267 - protein, 5 aa, Artificial sequence
  synthetic
DELLY

SEQ ID NO: 268 - protein, 5 aa, Artificial sequence
  synthetic
ELLYG

SEQ ID NO: 269 - protein, 5 aa, Artificial sequence
  synthetic
LLYGR

SEQ ID NO: 270 - protein, 5 aa, Artificial sequence
  synthetic
LYGRA

SEQ ID NO: 271 - protein, 5 aa, Artificial sequence
  synthetic
YGRAG

SEQ ID NO: 272 - protein, 5 aa, Artificial sequence
  synthetic
GRAGY

SEQ ID NO: 273 - protein, 5 aa, Artificial sequence
  synthetic
YLNTE

SEQ ID NO: 274 - protein, 5 aa, Artificial sequence
  synthetic
LNTEI

SEQ ID NO: 275 - protein, 5 aa, Artificial sequence
  synthetic
NTEIG

SEQ ID NO: 276 - protein, 5 aa, Artificial sequence
  synthetic
TEIGP

SEQ ID NO: 277 - protein, 5 aa, Artificial sequence
  synthetic
AGIYY

SEQ ID NO: 278 - protein, 5 aa, Artificial sequence
  synthetic
GIYYM

SEQ ID NO: 279 - protein, 5 aa, Artificial sequence
  synthetic
IYYML

SEQ ID NO: 280 - protein, 5 aa, Artificial sequence
  synthetic
YYMLM

SEQ ID NO: 281 - protein, 5 aa, Artificial sequence
  synthetic
YMLMQ

SEQ ID NO: 282 - protein, 5 aa, Artificial sequence
  synthetic
MLMQP

SEQ ID NO: 283 - protein, 5 aa, Artificial sequence
  synthetic
LMQPA

SEQ ID NO: 284 - protein, 5 aa, Artificial sequence
  synthetic
MQPAA

SEQ ID NO: 285 - protein, 5 aa, Artificial sequence
  synthetic
QPAAK

SEQ ID NO: 286 - protein, 5 aa, Artificial sequence
  synthetic
HWCHG

SEQ ID NO: 287 - protein, 5 aa, Artificial sequence
  synthetic
WCHGA

SEQ ID NO: 288 - protein, 5 aa, Artificial sequence
  synthetic
CHGAP

SEQ ID NO: 289 - protein, 5 aa, Artificial sequence
  synthetic
HGAPG

SEQ ID NO: 290 - protein, 5 aa, Artificial sequence
  synthetic
IHMLM

SEQ ID NO: 291 - protein, 5 aa, Artificial sequence
  synthetic
HMLMQ

SEQ ID NO: 292 - protein, 5 aa, Artificial sequence
  synthetic
MLMQA

SEQ ID NO: 293 - protein, 5 aa, Artificial sequence
  synthetic
LMQAY

SEQ ID NO: 294 - protein, 5 aa, Artificial sequence
  synthetic
DRPYS

SEQ ID NO: 295 - protein, 5 aa, Artificial sequence
  synthetic
RPYSL

SEQ ID NO: 296 - protein, 5 aa, Artificial sequence
  synthetic
PYSLF

SEQ ID NO: 297 - protein, 5 aa, Artificial sequence
  synthetic
YSLFE

SEQ ID NO: 298 - protein, 5 aa, Artificial sequence
  synthetic
SLFEG

SEQ ID NO: 299 - protein, 5 aa, Artificial sequence
  synthetic
LFEGM

SEQ ID NO: 300 - protein, 5 aa, Artificial sequence
  synthetic
FEGMA

SEQ ID NO: 301 - protein, 5 aa, Artificial sequence
  synthetic
EGMAG

SEQ ID NO: 302 - protein, 5 aa, Artificial sequence
  synthetic
GMAGA

SEQ ID NO: 303 - protein, 5 aa, Artificial sequence
  synthetic
MAGAI

SEQ ID NO: 304 - protein, 5 aa, Artificial sequence
  synthetic
AGAIH

SEQ ID NO: 305 - protein, 5 aa, Artificial sequence
  synthetic
GAIHF

SEQ ID NO: 306 - protein, 5 aa, Artificial sequence
  synthetic
AIHFL

SEQ ID NO: 307 - protein, 5 aa, Artificial sequence
  synthetic
IHFLS

SEQ ID NO: 308 - protein, 15 aa, Artificial sequence
  synthetic
SAYTGWTGIALLYLQ

SEQ ID NO: 309 - protein, 15 aa, Artificial sequence
  synthetic
AYTGWTGIALLYLQL

SEQ ID NO: 310 - protein, 15 aa, Artificial sequence
  synthetic
YTGWTGIALLYLQLY

SEQ ID NO: 311 - protein, 15 aa, Artificial sequence
  synthetic
TGWTGIALLYLQLYR

SEQ ID NO: 312 - protein, 15 aa, Artificial sequence
  synthetic
GWTGIALLYLQLYRV

SEQ ID NO: 313 - protein, 15 aa, Artificial sequence
  synthetic
WTGIALLYLQLYRVT

SEQ ID NO: 314 - protein, 15 aa, Artificial sequence
  synthetic
TGIALLYLQLYRVTC

SEQ ID NO: 315 - protein, 15 aa, Artificial sequence
  synthetic
GIALLYLQLYRVTCD

SEQ ID NO: 316 - protein, 15 aa, Artificial sequence
  synthetic
IALLYLQLYRVTCDQ

SEQ ID NO: 317 - protein, 15 aa, Artificial sequence
  synthetic
ALLYLQLYRVTCDQT

SEQ ID NO: 318 - protein, 15 aa, Artificial sequence
  synthetic
LLYLQLYRVTCDQTY

SEQ ID NO: 319 - protein, 15 aa, Artificial sequence
  synthetic
LYLQLYRVTCDQTYL

SEQ ID NO: 320 - protein, 15 aa, Artificial sequence
  synthetic
YLQLYRVTCDQTYLL

SEQ ID NO: 321 - protein, 15 aa, Artificial sequence
  synthetic
LQLYRVTCDQTYLLR

SEQ ID NO: 322 - protein, 15 aa, Artificial sequence
  synthetic
LPDELLYGRAGYLYA

SEQ ID NO: 323 - protein, 15 aa, Artificial sequence
  synthetic
PDELLYGRAGYLYAL

SEQ ID NO: 324 - protein, 15 aa, Artificial sequence
  synthetic
DELLYGRAGYLYALL

SEQ ID NO: 325 - protein, 15 aa, Artificial sequence
  synthetic
ELLYGRAGYLYALLY

SEQ ID NO: 326 - protein, 15 aa, Artificial sequence
  synthetic
LLYGRAGYLYALLYL

SEQ ID NO: 327 - protein, 15 aa, Artificial sequence
  synthetic
LYGRAGYLYALLYLN

SEQ ID NO: 328 - protein, 15 aa, Artificial sequence
  synthetic
YGRAGYLYALLYLNT

SEQ ID NO: 329 - protein, 15 aa, Artificial sequence
  synthetic
GRAGYLYALLYLNTE

SEQ ID NO: 330 - protein, 15 aa, Artificial sequence
  synthetic
RAGYLYALLYLNTEI

SEQ ID NO: 331 - protein, 15 aa, Artificial sequence
  synthetic
AGYLYALLYLNTEIG

SEQ ID NO: 332 - protein, 15 aa, Artificial sequence
  synthetic
GYLYALLYLNTEIGP

SEQ ID NO: 333 - protein, 15 aa, Artificial sequence
  synthetic
YLYALLYLNTEIGPG

SEQ ID NO: 334 - protein, 15 aa, Artificial sequence
  synthetic
LYALLYLNTEIGPGT

SEQ ID NO: 335 - protein, 15 aa, Artificial sequence
  synthetic
YALLYLNTEIGPGTV

SEQ ID NO: 336 - protein, 15 aa, Artificial sequence
  synthetic
ALLYLNTEIGPGTVC

SEQ ID NO: 337 - protein, 15 aa, Artificial sequence
  synthetic
LLYLNTEIGPGTVCE

SEQ ID NO: 338 - protein, 15 aa, Artificial sequence
  synthetic
AHGMAGIYYMLMQPA

SEQ ID NO: 339 - protein, 15 aa, Artificial sequence
  synthetic
HGMAGIYYMLMQPAA

SEQ ID NO: 340 - protein, 15 aa, Artificial sequence
  synthetic
GMAGIYYMLMQPAAK

SEQ ID NO: 341 - protein, 15 aa, Artificial sequence
  synthetic
MAGIYYMLMQPAAKV

SEQ ID NO: 342 - protein, 15 aa, Artificial sequence
  synthetic
AGIYYMLMQPAAKVD

SEQ ID NO: 343 - protein, 15 aa, Artificial sequence
  synthetic
GIYYMLMQPAAKVDQ

SEQ ID NO: 344 - protein, 15 aa, Artificial sequence
  synthetic
IYYMLMQPAAKVDQE

SEQ ID NO: 345 - protein, 15 aa, Artificial sequence
  synthetic
YYMLMQPAAKVDQET

SEQ ID NO: 346 - protein, 15 aa, Artificial sequence
  synthetic
RLVHWCHGAPGVIHM

SEQ ID NO: 347 - protein, 15 aa, Artificial sequence
  synthetic
LVHWCHGAPGVIHML

SEQ ID NO: 348 - protein, 15 aa, Artificial sequence
  synthetic
VHWCHGAPGVIHMLM

SEQ ID NO: 349 - protein, 15 aa, Artificial sequence
  synthetic
HWCHGAPGVIHMLMQ

SEQ ID NO: 350 - protein, 15 aa, Artificial sequence
  synthetic
WCHGAPGVIHMLMQA

SEQ ID NO: 351 - protein, 15 aa, Artificial sequence
  synthetic
CHGAPGVIHMLMQAY

SEQ ID NO: 352 - protein, 15 aa, Artificial sequence
  synthetic
HGAPGVIHMLMQAYK

SEQ ID NO: 353 - protein, 15 aa, Artificial sequence
  synthetic
GAPGVIHMLMQAYKV

SEQ ID NO: 354 - protein, 15 aa, Artificial sequence
  synthetic
APGVIHMLMQAYKVF

SEQ ID NO: 355 - protein, 15 aa, Artificial sequence
  synthetic
PGVIHMLMQAYKVFK

SEQ ID NO: 356 - protein, 15 aa, Artificial sequence
  synthetic
GVIHMLMQAYKVFKE

SEQ ID NO: 357 - protein, 15 aa, Artificial sequence
  synthetic
VIHMLMQAYKVFKEE

SEQ ID NO: 358 - protein, 15 aa, Artificial sequence
  synthetic
IHMLMQAYKVFKEEK

SEQ ID NO: 359 - protein, 15 aa, Artificial sequence
  synthetic
PDRPYSLFEGMAGAI

SEQ ID NO: 360 - protein, 15 aa, Artificial sequence
  synthetic
DRPYSLFEGMAGAIH

SEQ ID NO: 361 - protein, 15 aa, Artificial sequence
  synthetic
RPYSLFEGMAGAIHF

SEQ ID NO: 362 - protein, 15 aa, Artificial sequence
  synthetic
PYSLFEGMAGAIHFL

SEQ ID NO: 363 - protein, 15 aa, Artificial sequence
  synthetic
YSLFEGMAGAIHFLS

SEQ ID NO: 364 - protein, 15 aa, Artificial sequence
  synthetic
SLFEGMAGAIHFLSD

SEQ ID NO: 365 - protein, 15 aa, Artificial sequence
  synthetic
LFEGMAGAIHFLSDV

SEQ ID NO: 366 - protein, 15 aa, Artificial sequence
  synthetic
FEGMAGAIHFLSDVL

SEQ ID NO: 367 - protein, 15 aa, Artificial sequence
  synthetic
EGMAGAIHFLSDVLG

SEQ ID NO: 368 - protein, 15 aa, Artificial sequence
  synthetic
GMAGAIHFLSDVLGP

SEQ ID NO: 369 - protein, 15 aa, Artificial sequence
  synthetic
MAGAIHFLSDVLGPE

SEQ ID NO: 370 - protein, 15 aa, Artificial sequence
  synthetic
AGAIHFLSDVLGPET

SEQ ID NO: 371 - protein, 5 aa, Artificial sequence
  synthetic
GWGAL

SEQ ID NO: 372 - protein, 5 aa, Artificial sequence
  synthetic
WTILL

SEQ ID NO: 373 - protein, 5 aa, Artificial sequence
  synthetic
TGALY

SEQ ID NO: 374 - protein, 5 aa, Artificial sequence
  synthetic
GILYL

SEQ ID NO: 375 - protein, 5 aa, Artificial sequence
  synthetic
IALLQ

SEQ ID NO: 376 - protein, 5 aa, Artificial sequence
  synthetic
ALYQL

SEQ ID NO: 377 - protein, 5 aa, Artificial sequence
  synthetic
LLLLY

SEQ ID NO: 378 - protein, 5 aa, Artificial sequence
  synthetic
LYQYR

SEQ ID NO: 379 - protein, 5 aa, Artificial sequence
  synthetic
YLLRV

SEQ ID NO: 380 - protein, 5 aa, Artificial sequence
  synthetic
LQYVT

SEQ ID NO: 381 - protein, 5 aa, Artificial sequence
  synthetic
QLRTC

SEQ ID NO: 382 - protein, 5 aa, Artificial sequence
  synthetic
LYVCD

SEQ ID NO: 383 - protein, 5 aa, Artificial sequence
  synthetic
YRTDQ

SEQ ID NO: 384 - protein, 5 aa, Artificial sequence
  synthetic
RVCQT

SEQ ID NO: 385 - protein, 5 aa, Artificial sequence
  synthetic
LLGAG

SEQ ID NO: 386 - protein, 5 aa, Artificial sequence
  synthetic
LYRGY

SEQ ID NO: 387 - protein, 5 aa, Artificial sequence
  synthetic
YGAYL

SEQ ID NO: 388 - protein, 5 aa, Artificial sequence
  synthetic
GRGLY

SEQ ID NO: 389 - protein, 5 aa, Artificial sequence
  synthetic
RAYYA

SEQ ID NO: 390 - protein, 5 aa, Artificial sequence
  synthetic
AGLAL

SEQ ID NO: 391 - protein, 5 aa, Artificial sequence
  synthetic
GYYLL

SEQ ID NO: 392 - protein, 5 aa, Artificial sequence
  synthetic
YLALY

SEQ ID NO: 393 - protein, 5 aa, Artificial sequence
  synthetic
LYLYL

SEQ ID NO: 394 - protein, 5 aa, Artificial sequence
  synthetic
YALLN

SEQ ID NO: 395 - protein, 5 aa, Artificial sequence
  synthetic
ALYNT

SEQ ID NO: 396 - protein, 5 aa, Artificial sequence
  synthetic
LLLTE

SEQ ID NO: 397 - protein, 5 aa, Artificial sequence
  synthetic
LYNEI

SEQ ID NO: 398 - protein, 5 aa, Artificial sequence
  synthetic
YLTIG

SEQ ID NO: 399 - protein, 5 aa, Artificial sequence
  synthetic
LNEGP

SEQ ID NO: 400 - protein, 5 aa, Artificial sequence
  synthetic
NTIPG

SEQ ID NO: 401 - protein, 5 aa, Artificial sequence
  synthetic
AGYML

SEQ ID NO: 402 - protein, 5 aa, Artificial sequence
  synthetic
GIYLM

SEQ ID NO: 403 - protein, 5 aa, Artificial sequence
  synthetic
IYMMQ

SEQ ID NO: 404 - protein, 5 aa, Artificial sequence
  synthetic
YYLQP

SEQ ID NO: 405 - protein, 5 aa, Artificial sequence
  synthetic
YMMPA

SEQ ID NO: 406 - protein, 5 aa, Artificial sequence
  synthetic
MLQAA

SEQ ID NO: 407 - protein, 5 aa, Artificial sequence
  synthetic
LMPAK

SEQ ID NO: 408 - protein, 5 aa, Artificial sequence
  synthetic
MQAKV

SEQ ID NO: 409 - protein, 5 aa, Artificial sequence
  synthetic
WCGPG

SEQ ID NO: 410 - protein, 5 aa, Artificial sequence
  synthetic
CHAGV

SEQ ID NO: 411 - protein, 5 aa, Artificial sequence
  synthetic
HGPVI

SEQ ID NO: 412 - protein, 5 aa, Artificial sequence
  synthetic
GAGIH

SEQ ID NO: 413 - protein, 5 aa, Artificial sequence
  synthetic
APVHM

SEQ ID NO: 414 - protein, 5 aa, Artificial sequence
  synthetic
PGIML

SEQ ID NO: 415 - protein, 5 aa, Artificial sequence
  synthetic
GVHLM

SEQ ID NO: 416 - protein, 5 aa, Artificial sequence
  synthetic
VIMMQ

SEQ ID NO: 417 - protein, 5 aa, Artificial sequence
  synthetic
IHLQA

SEQ ID NO: 418 - protein, 5 aa, Artificial sequence
  synthetic
HMMAY

SEQ ID NO: 419 - protein, 5 aa, Artificial sequence
  synthetic
MLQYK

SEQ ID NO: 420 - protein, 5 aa, Artificial sequence
  synthetic
LMAKV

SEQ ID NO: 421 - protein, 5 aa, Artificial sequence
  synthetic
MQYVF

SEQ ID NO: 422 - protein, 5 aa, Artificial sequence
  synthetic
YSFGM

SEQ ID NO: 423 - protein, 5 aa, Artificial sequence
  synthetic
SLEMA

SEQ ID NO: 424 - protein, 5 aa, Artificial sequence
  synthetic
LFGAG

SEQ ID NO: 425 - protein, 5 aa, Artificial sequence
  synthetic
FEMGA

SEQ ID NO: 426 - protein, 5 aa, Artificial sequence
  synthetic
EGAAI

SEQ ID NO: 427 - protein, 5 aa, Artificial sequence
  synthetic
GMGIH

SEQ ID NO: 428 - protein, 5 aa, Artificial sequence
  synthetic
MAAHF

SEQ ID NO: 429 - protein, 5 aa, Artificial sequence
  synthetic
AGIFL

SEQ ID NO: 430 - protein, 5 aa, Artificial sequence
  synthetic
GAHLS

SEQ ID NO: 431 - protein, 5 aa, Artificial sequence
  synthetic
AIFSD

SEQ ID NO: 432 - protein, 5 aa, Artificial sequence
  synthetic
IHLDV

SEQ ID NO: 433 - protein, 5 aa, Artificial sequence
  synthetic
HFSVL

SEQ ID NO: 434 - protein, 9 aa, Artificial sequence
  synthetic
IYWGRAGYV

SEQ ID NO: 435 - protein, 9 aa, Artificial sequence
  synthetic
HLYGRAGYV

SEQ ID NO: 436 - protein, 9 aa, Artificial sequence
  synthetic
MGWGRAGYL

SEQ ID NO: 437 - protein, 9 aa, Artificial sequence
  synthetic
YPWGRAGYL

SEQ ID NO: 438 - protein, 9 aa, Artificial sequence
  synthetic
LGDGRAGYW

SEQ ID NO: 439 - protein, 9 aa, Artificial sequence
  synthetic
KGRGRAGYW

SEQ ID NO: 440 - protein, 9 aa, Artificial sequence
  synthetic
WVKGRAGYR

SEQ ID NO: 441 - protein, 9 aa, Artificial sequence
  synthetic
RVKGRAGYL

SEQ ID NO: 442 - protein, 9 aa, Artificial sequence
  synthetic
FAAEGMAGR

SEQ ID NO: 443 - protein, 9 aa, Artificial sequence
  synthetic
PVAEGMAGR

SEQ ID NO: 444 - protein, 9 aa, Artificial sequence
  synthetic
GIVEGMAGR

SEQ ID NO: 445 - protein, 9 aa, Artificial sequence
  synthetic
FVGEGMAGR

SEQ ID NO: 446 - protein, 9 aa, Artificial sequence
  synthetic
HMLEGMAGK

SEQ ID NO: 447 - protein, 9 aa, Artificial sequence
  synthetic
LFGEGMAGR

SEQ ID NO: 448 - protein, 9 aa, Artificial sequence
  synthetic
ALIEGMAGR

SEQ ID NO: 449 - protein, 9 aa, Artificial sequence
  synthetic
SFLEGMAGR

SEQ ID NO: 450 - protein, 9 aa, Artificial sequence
  synthetic
QEAFEGMAR

SEQ ID NO: 451 - protein, 9 aa, Artificial sequence
  synthetic
YDQFEGMAR

SEQ ID NO: 452 - protein, 9 aa, Artificial sequence
  synthetic
EYSFEGMAK

SEQ ID NO: 453 - protein, 9 aa, Artificial sequence
  synthetic
YQEFEGMAR

SEQ ID NO: 454 - protein, 5 aa, Artificial sequence
  synthetic
GRAGY

SEQ ID NO: 455 - protein, 5 aa, Artificial sequence
  synthetic
EGMAG

SEQ ID NO: 456 - protein, 5 aa, Artificial sequence
  synthetic
FEGMA

SEQ ID NO: 457 - protein, 9 aa, Artificial sequence
  synthetic
IRQGFTFNI

SEQ ID NO: 458 - protein, 9 aa, Artificial sequence
  synthetic
RQGFTFNIL

SEQ ID NO: 459 - protein, 9 aa, Artificial sequence
  synthetic
QGFTFNILC

SEQ ID NO: 460 - protein, 9 aa, Artificial sequence
  synthetic
GFTFNILCV

SEQ ID NO: 461 - protein, 9 aa, Artificial sequence
  synthetic
FTFNILCVG

SEQ ID NO: 462 - protein, 9 aa, Artificial sequence
  synthetic
TFNILCVGE

SEQ ID NO: 463 - protein, 9 aa, Artificial sequence
  synthetic
FNILCVGET

SEQ ID NO: 464 - protein, 9 aa, Artificial sequence
  synthetic
NILCVGETG

SEQ ID NO: 465 - protein, 9 aa, Artificial sequence
  synthetic
ILCVGETGI

SEQ ID NO: 466 - protein, 9 aa, Artificial sequence
  synthetic
LCVGETGIG

SEQ ID NO: 467 - protein, 9 aa, Artificial sequence
  synthetic
RVHVCLYFI

SEQ ID NO: 468 - protein, 9 aa, Artificial sequence
  synthetic
VHVCLYFIS

SEQ ID NO: 469 - protein, 9 aa, Artificial sequence
  synthetic
HVCLYFISP

SEQ ID NO: 470 - protein, 9 aa, Artificial sequence
  synthetic
VCLYFISPT

SEQ ID NO: 471 - protein, 9 aa, Artificial sequence
  synthetic
CLYFISPTG

SEQ ID NO: 472 - protein, 9 aa, Artificial sequence
  synthetic
LYFISPTGH

SEQ ID NO: 473 - protein, 9 aa, Artificial sequence
  synthetic
YFISPTGHS

SEQ ID NO: 474 - protein, 9 aa, Artificial sequence
  synthetic
FISPTGHSL

SEQ ID NO: 475 - protein, 9 aa, Artificial sequence
  synthetic
NLDSKVNII

SEQ ID NO: 476 - protein, 9 aa, Artificial sequence
  synthetic
LDSKVNIIP

SEQ ID NO: 477 - protein, 9 aa, Artificial sequence
  synthetic
DSKVNIIPL

SEQ ID NO: 478 - protein, 9 aa, Artificial sequence
  synthetic
SKVNIIPLI

SEQ ID NO: 479 - protein, 9 aa, Artificial sequence
  synthetic
KVNIIPLIA

SEQ ID NO: 480 - protein, 9 aa, Artificial sequence
  synthetic
VNIIPLIAK

SEQ ID NO: 481 - protein, 9 aa, Artificial sequence
  synthetic
NIIPLIAKA

SEQ ID NO: 482 - protein, 9 aa, Artificial sequence
  synthetic
IIPLIAKAD

SEQ ID NO: 483 - protein, 9 aa, Artificial sequence
  synthetic
IPLIAKADT

SEQ ID NO: 484 - protein, 9 aa, Artificial sequence
  synthetic
PLIAKADTI

SEQ ID NO: 485 - protein, 9 aa, Artificial sequence
  synthetic
LIAKADTIS

SEQ ID NO: 486 - protein, 9 aa, Artificial sequence
  synthetic
PLIAKADTI

SEQ ID NO: 487 - protein, 9 aa, Artificial sequence
  synthetic
LIAKADTIS

SEQ ID NO: 488 - protein, 5 aa, Artificial sequence
  synthetic
GFTFN

SEQ ID NO: 489 - protein, 5 aa, Artificial sequence
  synthetic
FTFNI

SEQ ID NO: 490 - protein, 5 aa, Artificial sequence
  synthetic
TFNIL

SEQ ID NO: 491 - protein, 5 aa, Artificial sequence
  synthetic
FNILC

SEQ ID NO: 492 - protein, 5 aa, Artificial sequence
  synthetic
NILCV

SEQ ID NO: 493 - protein, 5 aa, Artificial sequence
  synthetic
ILCVG

SEQ ID NO: 494 - protein, 5 aa, Artificial sequence
  synthetic
LCVGE

SEQ ID NO: 495 - protein, 5 aa, Artificial sequence
  synthetic
CVGET

SEQ ID NO: 496 - protein, 5 aa, Artificial sequence
  synthetic
VGETG

SEQ ID NO: 497 - protein, 5 aa, Artificial sequence
  synthetic
GETGI

SEQ ID NO: 498 - protein, 5 aa, Artificial sequence
  synthetic
VCLYF

SEQ ID NO: 499 - protein, 5 aa, Artificial sequence
  synthetic
CLYFI

SEQ ID NO: 500 - protein, 5 aa, Artificial sequence
  synthetic
LYFIS

SEQ ID NO: 501 - protein, 5 aa, Artificial sequence
  synthetic
YFISP

SEQ ID NO: 502 - protein, 5 aa, Artificial sequence
  synthetic
FISPT

SEQ ID NO: 503 - protein, 5 aa, Artificial sequence
  synthetic
ISPTG

SEQ ID NO: 504 - protein, 5 aa, Artificial sequence
  synthetic
SPTGH

SEQ ID NO: 505 - protein, 5 aa, Artificial sequence
  synthetic
PTGHS

SEQ ID NO: 506 - protein, 5 aa, Artificial sequence
  synthetic
SKVNI

SEQ ID NO: 507 - protein, 5 aa, Artificial sequence
  synthetic
KVNII

SEQ ID NO: 508 - protein, 5 aa, Artificial sequence
  synthetic
VNIIP

SEQ ID NO: 509 - protein, 5 aa, Artificial sequence
  synthetic
NIIPL

SEQ ID NO: 510 - protein, 5 aa, Artificial sequence
  synthetic
IIPLI

SEQ ID NO: 511 - protein, 5 aa, Artificial sequence
  synthetic
IPLIA

SEQ ID NO: 512 - protein, 5 aa, Artificial sequence
  synthetic
PLIAK

SEQ ID NO: 513 - protein, 5 aa, Artificial sequence
  synthetic
LIAKA

SEQ ID NO: 514 - protein, 5 aa, Artificial sequence
  synthetic
IAKAD

SEQ ID NO: 515 - protein, 5 aa, Artificial sequence
  synthetic
AKADT

SEQ ID NO: 516 - protein, 5 aa, Artificial sequence
  synthetic
KADTI

SEQ ID NO: 517 - protein, 5 aa, Artificial sequence
  synthetic
GFTFN

SEQ ID NO: 518 - protein, 5 aa, Artificial sequence
  synthetic
FTFNI

SEQ ID NO: 519 - protein, 5 aa, Artificial sequence
  synthetic
TFNIL

SEQ ID NO: 520 - protein, 5 aa, Artificial sequence
  synthetic
FNILC

SEQ ID NO: 521 - protein, 5 aa, Artificial sequence
  synthetic
NILCV

SEQ ID NO: 522 - protein, 5 aa, Artificial sequence
  synthetic
ILCVG

SEQ ID NO: 523 - protein, 5 aa, Artificial sequence
  synthetic
LCVGE

SEQ ID NO: 524 - protein, 5 aa, Artificial sequence
  synthetic
CVGET

SEQ ID NO: 525 - protein, 5 aa, Artificial sequence
  synthetic
VGETG

SEQ ID NO: 526 - protein, 5 aa, Artificial sequence
  synthetic
GETGI

SEQ ID NO: 527 - protein, 5 aa, Artificial sequence
  synthetic
VCLYF

SEQ ID NO: 528 - protein, 5 aa, Artificial sequence
  synthetic
CLYFI

SEQ ID NO: 529 - protein, 5 aa, Artificial sequence
  synthetic
LYFIS

SEQ ID NO: 530 - protein, 5 aa, Artificial sequence
  synthetic
YFISP

SEQ ID NO: 531 - protein, 5 aa, Artificial sequence
  synthetic
FISPT

SEQ ID NO: 532 - protein, 5 aa, Artificial sequence
  synthetic
ISPTG

SEQ ID NO: 533 - protein, 5 aa, Artificial sequence
  synthetic
SPTGH

SEQ ID NO: 534 - protein, 5 aa, Artificial sequence
  synthetic
PTGHS

SEQ ID NO: 535 - protein, 5 aa, Artificial sequence
  synthetic
SKVNI

SEQ ID NO: 536 - protein, 5 aa, Artificial sequence
  synthetic
KVNII

SEQ ID NO: 537 - protein, 5 aa, Artificial sequence
  synthetic
VNIIP

SEQ ID NO: 538 - protein, 5 aa, Artificial sequence
  synthetic
NIIPL

SEQ ID NO: 539 - protein, 5 aa, Artificial sequence
  synthetic
IIPLI

SEQ ID NO: 540 - protein, 5 aa, Artificial sequence
  synthetic
IPLIA

SEQ ID NO: 541 - protein, 5 aa, Artificial sequence
  synthetic
PLIAK

SEQ ID NO: 542 - protein, 5 aa, Artificial sequence
  synthetic
LIAKA

SEQ ID NO: 543 - protein, 5 aa, Artificial sequence
  synthetic
IAKAD

SEQ ID NO: 544 - protein, 5 aa, Artificial sequence
  synthetic
AKADT

SEQ ID NO: 545 - protein, 5 aa, Artificial sequence
  synthetic
KADTI

SEQ ID NO: 546 - protein, 15 aa, Artificial sequence
  synthetic
QLVSRSIRQGFTFNI

SEQ ID NO: 547 - protein, 15 aa, Artificial sequence
  synthetic
LVSRSIRQGFTFNIL

SEQ ID NO: 548 - protein, 15 aa, Artificial sequence
  synthetic
VSRSIRQGFTFNILC

SEQ ID NO: 549 - protein, 15 aa, Artificial sequence
  synthetic
SRSIRQGFTFNILCV

SEQ ID NO: 550 - protein, 15 aa, Artificial sequence
  synthetic
RSIRQGFTFNILCVG

SEQ ID NO: 551 - protein, 15 aa, Artificial sequence
  synthetic
SIRQGFTFNILCVGE

SEQ ID NO: 552 - protein, 15 aa, Artificial sequence
  synthetic
IRQGFTFNILCVGET

SEQ ID NO: 553 - protein, 15 aa, Artificial sequence
  synthetic
RQGFTFNILCVGETG

SEQ ID NO: 554 - protein, 15 aa, Artificial sequence
  synthetic
QGFTFNILCVGETGI

SEQ ID NO: 555 - protein, 15 aa, Artificial sequence
  synthetic
GFTFNILCVGETGIG

SEQ ID NO: 556 - protein, 15 aa, Artificial sequence
  synthetic
FTFNILCVGETGIGK

SEQ ID NO: 557 - protein, 15 aa, Artificial sequence
  synthetic
TFNILCVGETGIGKS

SEQ ID NO: 558 - protein, 15 aa, Artificial sequence
  synthetic
HDSRVHVCLYFISPT

SEQ ID NO: 559 - protein, 15 aa, Artificial sequence
  synthetic
DSRVHVCLYFISPTG

SEQ ID NO: 560 - protein, 15 aa, Artificial sequence
  synthetic
SRVHVCLYFISPTGH

SEQ ID NO: 561 - protein, 15 aa, Artificial sequence
  synthetic
RVHVCLYFISPTGHS

SEQ ID NO: 562 - protein, 15 aa, Artificial sequence
  synthetic
VHVCLYFISPTGHSL

SEQ ID NO: 563 - protein, 15 aa, Artificial sequence
  synthetic
HVCLYFISPTGHSLK

SEQ ID NO: 564 - protein, 15 aa, Artificial sequence
  synthetic
VCLYFISPTGHSLKS

SEQ ID NO: 565 - protein, 15 aa, Artificial sequence
  synthetic
CLYFISPTGHSLKSL

SEQ ID NO: 566 - protein, 15 aa, Artificial sequence
  synthetic
LYFISPTGHSLKSLD

SEQ ID NO: 567 - protein, 15 aa, Artificial sequence
  synthetic
NLDSKVNIIPLIAKA

SEQ ID NO: 568 - protein, 15 aa, Artificial sequence
  synthetic
LDSKVNIIPLIAKAD

SEQ ID NO: 569 - protein, 15 aa, Artificial sequence
  synthetic
DSKVNIIPLIAKADT

SEQ ID NO: 570 - protein, 15 aa, Artificial sequence
  synthetic
SKVNIIPLIAKADTI

SEQ ID NO: 571 - protein, 15 aa, Artificial sequence
  synthetic
KVNIIPLIAKADTIS

SEQ ID NO: 572 - protein, 15 aa, Artificial sequence
  synthetic
VNIIPLIAKADTISK

SEQ ID NO: 573 - protein, 15 aa, Artificial sequence
  synthetic
NIIPLIAKADTISKN

SEQ ID NO: 574 - protein, 15 aa, Artificial sequence
  synthetic
IIPLIAKADTISKND

SEQ ID NO: 575 - protein, 5 aa, Artificial sequence
  synthetic
RSRGF

SEQ ID NO: 576 - protein, 5 aa, Artificial sequence
  synthetic
SIQFT

SEQ ID NO: 577 - protein, 5 aa, Artificial sequence
  synthetic
IRGTF

SEQ ID NO: 578 - protein, 5 aa, Artificial sequence
  synthetic
RQFFN

SEQ ID NO: 579 - protein, 5 aa, Artificial sequence
  synthetic
QGTNI

SEQ ID NO: 580 - protein, 5 aa, Artificial sequence
  synthetic
GFFIL

SEQ ID NO: 581 - protein, 5 aa, Artificial sequence
  synthetic
FTNLC

SEQ ID NO: 582 - protein, 5 aa, Artificial sequence
  synthetic
TFICV

SEQ ID NO: 583 - protein, 5 aa, Artificial sequence
  synthetic
FNLVG

SEQ ID NO: 584 - protein, 5 aa, Artificial sequence
  synthetic
NICGE

SEQ ID NO: 585 - protein, 5 aa, Artificial sequence
  synthetic
ILVET

SEQ ID NO: 586 - protein, 5 aa, Artificial sequence
  synthetic
LCGTG

SEQ ID NO: 587 - protein, 5 aa, Artificial sequence
  synthetic
VHCYF

SEQ ID NO: 588 - protein, 5 aa, Artificial sequence
  synthetic
HVLFI

SEQ ID NO: 589 - protein, 5 aa, Artificial sequence
  synthetic
VCYIS

SEQ ID NO: 590 - protein, 5 aa, Artificial sequence
  synthetic
CLFSP

SEQ ID NO: 591 - protein, 5 aa, Artificial sequence
  synthetic
LYIPT

SEQ ID NO: 592 - protein, 5 aa, Artificial sequence
  synthetic
YFSTG

SEQ ID NO: 593 - protein, 5 aa, Artificial sequence
  synthetic
FIPGH

SEQ ID NO: 594 - protein, 5 aa, Artificial sequence
  synthetic
ISTHS

SEQ ID NO: 595 - protein, 5 aa, Artificial sequence
  synthetic
SPGSL

SEQ ID NO: 596 - protein, 5 aa, Artificial sequence
  synthetic
KVIPL

SEQ ID NO: 597 - protein, 5 aa, Artificial sequence
  synthetic
VNILI

SEQ ID NO: 598 - protein, 5 aa, Artificial sequence
  synthetic
NIPIA

SEQ ID NO: 599 - protein, 5 aa, Artificial sequence
  synthetic
IILAK

SEQ ID NO: 600 - protein, 5 aa, Artificial sequence
  synthetic
IPIKA

SEQ ID NO: 601 - protein, 5 aa, Artificial sequence
  synthetic
PLAAD

SEQ ID NO: 602 - protein, 5 aa, Artificial sequence
  synthetic
LIKDT

SEQ ID NO: 603 - protein, 5 aa, Artificial sequence
  synthetic
IAATI

SEQ ID NO: 604 - protein, 5 aa, Artificial sequence
  synthetic
YSFGD

SEQ ID NO: 605 - protein, 5 aa, Artificial sequence
  synthetic
SFGDT

SEQ ID NO: 606 - protein, 5 aa, Artificial sequence
  synthetic
FGDTC

SEQ ID NO: 607 - protein, 5 aa, Artificial sequence
  synthetic
GDTCV

SEQ ID NO: 608 - protein, 5 aa, Artificial sequence
  synthetic
DTCVK

SEQ ID NO: 609 - protein, 5 aa, Artificial sequence
  synthetic
YSFGV

SEQ ID NO: 610 - protein, 5 aa, Artificial sequence
  synthetic
SFGVT

SEQ ID NO: 611 - protein, 5 aa, Artificial sequence
  synthetic
FGVTC

SEQ ID NO: 612 - protein, 5 aa, Artificial sequence
  synthetic
GVTCV

SEQ ID NO: 613 - protein, 5 aa, Artificial sequence
  synthetic
VTCVK

SEQ ID NO: 614 - protein, 5 aa, Artificial sequence
  synthetic
YSFGT

SEQ ID NO: 615 - protein, 5 aa, Artificial sequence
  synthetic
SFGTT

SEQ ID NO: 616 - protein, 5 aa, Artificial sequence
  synthetic
FGTTC

SEQ ID NO: 617 - protein, 5 aa, Artificial sequence
  synthetic
GTTCV

SEQ ID NO: 618 - protein, 5 aa, Artificial sequence
  synthetic
TTCVK

SEQ ID NO: 619 - protein, 5 aa, Artificial sequence
  synthetic
TCPAD

SEQ ID NO: 620 - protein, 5 aa, Artificial sequence
  synthetic
CPADV

SEQ ID NO: 621 - protein, 5 aa, Artificial sequence
  synthetic
PADVM

SEQ ID NO: 622 - protein, 5 aa, Artificial sequence
  synthetic
ADVMG

SEQ ID NO: 623 - protein, 5 aa, Artificial sequence
  synthetic
DVMGE

SEQ ID NO: 624 - protein, 5 aa, Artificial sequence
  synthetic
TCPAV

SEQ ID NO: 625 - protein, 5 aa, Artificial sequence
  synthetic
CPAVV

SEQ ID NO: 626 - protein, 5 aa, Artificial sequence
  synthetic
PAVVM

SEQ ID NO: 627 - protein, 5 aa, Artificial sequence
  synthetic
AVVMG

SEQ ID NO: 628 - protein, 5 aa, Artificial sequence
  synthetic
VVMGE

SEQ ID NO: 629 - protein, 5 aa, Artificial sequence
  synthetic
TDFGR

SEQ ID NO: 630 - protein, 5 aa, Artificial sequence
  synthetic
DFGRA

SEQ ID NO: 631 - protein, 5 aa, Artificial sequence
  synthetic
FGRAK

SEQ ID NO: 632 - protein, 5 aa, Artificial sequence
  synthetic
GRAKL

SEQ ID NO: 633 - protein, 5 aa, Artificial sequence
  synthetic
RAKLL

SEQ ID NO: 634 - protein, 5 aa, Artificial sequence
  synthetic
EEKKG

SEQ ID NO: 635 - protein, 5 aa, Artificial sequence
  synthetic
EKKGN

SEQ ID NO: 636 - protein, 5 aa, Artificial sequence
  synthetic
KKGNY

SEQ ID NO: 637 - protein, 5 aa, Artificial sequence
  synthetic
KGNYV

SEQ ID NO: 638 - protein, 5 aa, Artificial sequence
  synthetic
GNYVV

SEQ ID NO: 639 - protein, 5 aa, Artificial sequence
  synthetic
GKSGD

SEQ ID NO: 640 - protein, 5 aa, Artificial sequence
  synthetic
KYFDT

SEQ ID NO: 641 - protein, 5 aa, Artificial sequence
  synthetic
SFDCV

SEQ ID NO: 642 - protein, 5 aa, Artificial sequence
  synthetic
GDCKK

SEQ ID NO: 643 - protein, 5 aa, Artificial sequence
  synthetic
DTVKC

SEQ ID NO: 644 - protein, 5 aa, Artificial sequence
  synthetic
GKSGV

SEQ ID NO: 645 - protein, 5 aa, Artificial sequence
  synthetic
KYFVT

SEQ ID NO: 646 - protein, 5 aa, Artificial sequence
  synthetic
SFVCV

SEQ ID NO: 647 - protein, 5 aa, Artificial sequence
  synthetic
GVCKK

SEQ ID NO: 648 - protein, 5 aa, Artificial sequence
  synthetic
VTVKC

SEQ ID NO: 649 - protein, 5 aa, Artificial sequence
  synthetic
GKSGT

SEQ ID NO: 650 - protein, 5 aa, Artificial sequence
  synthetic
KYFTT

SEQ ID NO: 651 - protein, 5 aa, Artificial sequence
  synthetic
SFTCV

SEQ ID NO: 652 - protein, 5 aa, Artificial sequence
  synthetic
GTCKK

SEQ ID NO: 653 - protein, 5 aa, Artificial sequence
  synthetic
TTVKC

SEQ ID NO: 654 - protein, 5 aa, Artificial sequence
  synthetic
VKCAD

SEQ ID NO: 655 - protein, 5 aa, Artificial sequence
  synthetic
KTPDV

SEQ ID NO: 656 - protein, 5 aa, Artificial sequence
  synthetic
CPDMG

SEQ ID NO: 657 - protein, 5 aa, Artificial sequence
  synthetic
ADMEN

SEQ ID NO: 658 - protein, 5 aa, Artificial sequence
  synthetic
DVGNN

SEQ ID NO: 659 - protein, 5 aa, Artificial sequence
  synthetic
VKCAV

SEQ ID NO: 660 - protein, 5 aa, Artificial sequence
  synthetic
KTPVV

SEQ ID NO: 661 - protein, 5 aa, Artificial sequence
  synthetic
CPVMG

SEQ ID NO: 662 - protein, 5 aa, Artificial sequence
  synthetic
AVMEN

SEQ ID NO: 663 - protein, 5 aa, Artificial sequence
  synthetic
VVGNN

SEQ ID NO: 664 - protein, 5 aa, Artificial sequence
  synthetic
KIDGR

SEQ ID NO: 665 - protein, 5 aa, Artificial sequence
  synthetic
ITFRA

SEQ ID NO: 666 - protein, 5 aa, Artificial sequence
  synthetic
DFRKL

SEQ ID NO: 667 - protein, 5 aa, Artificial sequence
  synthetic
GRKLG

SEQ ID NO: 668 - protein, 5 aa, Artificial sequence
  synthetic
RALGA

SEQ ID NO: 669 - protein, 5 aa, Artificial sequence
  synthetic
ALEKG

SEQ ID NO: 670 - protein, 5 aa, Artificial sequence
  synthetic
LEKGN

SEQ ID NO: 671 - protein, 5 aa, Artificial sequence
  synthetic
EKGYV

SEQ ID NO: 672 - protein, 5 aa, Artificial sequence
  synthetic
KGYVT

SEQ ID NO: 673 - protein, 5 aa, Artificial sequence
  synthetic
GNVTD

SEQ ID NO: 674 - protein, 5 aa, Artificial sequence
  synthetic
PKSGD

SEQ ID NO: 675 - protein, 5 aa, Artificial sequence
  synthetic
EYFDT

SEQ ID NO: 676 - protein, 5 aa, Artificial sequence
  synthetic
KFDCV

SEQ ID NO: 677 - protein, 5 aa, Artificial sequence
  synthetic
SDCKK

SEQ ID NO: 678 - protein, 5 aa, Artificial sequence
  synthetic
DVKPR

SEQ ID NO: 679 - protein, 5 aa, Artificial sequence
  synthetic
PKSGV

SEQ ID NO: 680 - protein, 5 aa, Artificial sequence
  synthetic
EYFVT

SEQ ID NO: 681 - protein, 5 aa, Artificial sequence
  synthetic
KFVCV

SEQ ID NO: 682 - protein, 5 aa, Artificial sequence
  synthetic
SVCKK

SEQ ID NO: 683 - protein, 5 aa, Artificial sequence
  synthetic
VVKPR

SEQ ID NO: 684 - protein, 5 aa, Artificial sequence
  synthetic
PKSGT

SEQ ID NO: 685 - protein, 5 aa, Artificial sequence
  synthetic
EYFTT

SEQ ID NO: 686 - protein, 5 aa, Artificial sequence
  synthetic
KFTCV

SEQ ID NO: 687 - protein, 5 aa, Artificial sequence
  synthetic
STCKK

SEQ ID NO: 688 - protein, 5 aa, Artificial sequence
  synthetic
TVKPR

SEQ ID NO: 689 - protein, 5 aa, Artificial sequence
  synthetic
HKCAD

SEQ ID NO: 690 - protein, 5 aa, Artificial sequence
  synthetic
CTPDV

SEQ ID NO: 691 - protein, 5 aa, Artificial sequence
  synthetic
KPDMG

SEQ ID NO: 692 - protein, 5 aa, Artificial sequence
  synthetic
CDMEN

SEQ ID NO: 693 - protein, 5 aa, Artificial sequence
  synthetic
DGNTL

SEQ ID NO: 694 - protein, 5 aa, Artificial sequence
  synthetic
HKCAV

SEQ ID NO: 695 - protein, 5 aa, Artificial sequence
  synthetic
CTPVV

SEQ ID NO: 696 - protein, 5 aa, Artificial sequence
  synthetic
KPVMG

SEQ ID NO: 697 - protein, 5 aa, Artificial sequence
  synthetic
CVMEN

SEQ ID NO: 698 - protein, 5 aa, Artificial sequence
  synthetic
VGNTL

SEQ ID NO: 699 - protein, 5 aa, Artificial sequence
  synthetic
HIDGR

SEQ ID NO: 700 - protein, 5 aa, Artificial sequence
  synthetic
VTFRA

SEQ ID NO: 701 - protein, 5 aa, Artificial sequence
  synthetic
IFRKL

SEQ ID NO: 702 - protein, 5 aa, Artificial sequence
  synthetic
DRKLG

SEQ ID NO: 703 - protein, 5 aa, Artificial sequence
  synthetic
RLGEE

SEQ ID NO: 704 - protein, 5 aa, Artificial sequence
  synthetic
SLEKG

SEQ ID NO: 705 - protein, 5 aa, Artificial sequence
  synthetic
REKGN

SEQ ID NO: 706 - protein, 5 aa, Artificial sequence
  synthetic
LKGYV

SEQ ID NO: 707 - protein, 5 aa, Artificial sequence
  synthetic
EGYVT

SEQ ID NO: 708 - protein, 5 aa, Artificial sequence
  synthetic
GVTHG

SEQ ID NO: 709 - protein, 5 aa, Artificial sequence
  synthetic
EEKKG

SEQ ID NO: 710 - protein, 5 aa, Artificial sequence
  synthetic
EKKGN

SEQ ID NO: 711 - protein, 5 aa, Artificial sequence
  synthetic
KKGNY

SEQ ID NO: 712 - protein, 5 aa, Artificial sequence
  synthetic
KGNYV

SEQ ID NO: 713 - protein, 9 aa, Artificial sequence
  synthetic
RALEEKKGN

SEQ ID NO: 714 - protein, 9 aa, Artificial sequence
  synthetic
ALEEKKGNY

SEQ ID NO: 715 - protein, 9 aa, Artificial sequence
  synthetic
LEEKKGNYV

SEQ ID NO: 716 - protein, 9 aa, Artificial sequence
  synthetic
EEKKGNYVV

SEQ ID NO: 717 - protein, 9 aa, Artificial sequence
  synthetic
RPAEEKKGK

SEQ ID NO: 718 - protein, 9 aa, Artificial sequence
  synthetic
RFAEEKKGK

SEQ ID NO: 719 - protein, 9 aa, Artificial sequence
  synthetic
FDSEEKKGW

SEQ ID NO: 720 - protein, 9 aa, Artificial sequence
  synthetic
WACEEKKGG

SEQ ID NO: 721 - protein, 9 aa, Artificial sequence
  synthetic
FTPEKKGNK

SEQ ID NO: 722 - protein, 9 aa, Artificial sequence
  synthetic
KSDEKKGNK

SEQ ID NO: 723 - protein, 9 aa, Artificial sequence
  synthetic
RLAEKKGNY

SEQ ID NO: 724 - protein, 9 aa, Artificial sequence
  synthetic
NVWEKKGNR

SEQ ID NO: 725 - protein, 9 aa, Artificial sequence
  synthetic
WSEEKKGNL

SEQ ID NO: 726 - protein, 9 aa, Artificial sequence
  synthetic
FVKEKKGNY

SEQ ID NO: 727 - protein, 9 aa, Artificial sequence
  synthetic
SFEEKKGNL

SEQ ID NO: 728 - protein, 9 aa, Artificial sequence
  synthetic
PFEEKKGNA

SEQ ID NO: 729 - protein, 9 aa, Artificial sequence
  synthetic
RCEKKGNYS

SEQ ID NO: 730 - protein, 9 aa, Artificial sequence
  synthetic
TEPKKGNYV

SEQ ID NO: 731 - protein, 9 aa, Artificial sequence
  synthetic
YCSKGNYVA

SEQ ID NO: 732 - protein, 9 aa, Artificial sequence
  synthetic
AESKGNYVK

SEQ ID NO: 733 - protein, 9 aa, Artificial sequence
  synthetic
LEPKGNYVS

SEQ ID NO: 734 - protein, 9 aa, Artificial sequence
  synthetic
WKYKGNYVG

(19) United States
(12) Patent Application Publication
(10) Pub. No.: US 2024/0016887 A1
(43) Pub. Date: Jan. 18, 2024

(54) BYSTANDER PROTEIN VACCINES

(71) Applicant: IOGENETICS, LLC, Madison, WI (US)

(72) Inventors: Jane Homan, Marshall, WI (US); Robert D. Bremel, Marshall, WI (US)

(21) Appl. No.: 18/256,241

(22) PCT Filed: Dec. 7, 2021
(86) PCT No.: PCT/US2021/062137

§ 371 Date: Jun. 7, 2023

Related U.S. Application Data
(60) Provisional application No. 63/122,191, filed on Dec. 7, 2020.

(51) Int. Cl. A61K 38/17; C12Q 1/6872; C07K 14/00

(52) U.S. Cl. CPC A61K 38/1709; C12Q 1/6872; C07K 14/001

(57) ABSTRACT

The present invention is related to T cell epitopes and methods of their use, in particular bystander proteins, and identification of peptides which may be used to stimulate a CD8+ cytotoxic T cell response, as well as peptides which stimulate a CD4+ helper T cell response to the cells carrying the proteins.

### FIELD OF THE INVENTION

The present invention is related to T cell epitopes and methods of their use, in particular bystander proteins, and identification of peptides which may be used to stimulate a CD8+ cytotoxic T cell response, as well as peptides which stimulate a CD4+ helper T cell response to the cells carrying the proteins.

### BACKGROUND OF THE INVENTION

There are a large number of well recognized oncogenes that play an important role in tumorigenesis as both drivers of tumor growth and as suppressors which may be silenced to enable tumor growth [1]. Focal amplification and gene rearrangements are characteristics of many cancer types [2, 3]. Sequence analysis of tumor biopsies in comparison to normal tissue identifies oncogenes that are upregulated, mutated, and increased in copy number in tumors.

There has been increasing interest in targeting these with neoepitope vaccines. In some instances, and particularly where oncogene amplification is the result of multiplication of extrachromosomal DNA, genes encoded in close proximity of oncogenes are also upregulated and their protein products expressed. While not mutated, the proteins derived from these bystander genes may be prognostic indicators. In the present invention we address the potential to target immune responses to the bystander gene products as a means to target and eliminate the tumor cell. Where bystander genes are carried on extrachromosomal DNA they may occur in different combinations, and may vary in relative level of expression between different clonal lines of a tumor. However, in so far as they are expressed as companions to the oncogene they provide markers of the cells in which the oncogene is upregulated.

### SUMMARY OF THE INVENTION

The present invention derives from the observation that upregulation of an oncogene may be accompanied by upregulation of proteins that are encoded in immediately adjacent or on the opposite DNA strand of sequences of the same chromosome and that such upregulated bystander proteins constitute targets to which a T cell response can be directed to eliminate the cancer cell. In some embodiments therefore this invention provides a method for sequencing the nucleic acids and proteins found in a tumor biopsy and comparing them to those in a normal tissue sample from the same subject, identifying those oncogenes which are increased in copy number and upregulated and determining which bystander proteins are associated with the oncogene having increased copy number and identifying the T cell epitopes in the bystander protein. In embodiments of the invention the predicted MHC binding affinity of peptides in the bystander protein is determined, as are the T cell exposed motifs comprised in such peptides. In some embodiments, peptides of a desired MHC binding affinity are selected and one or more such peptides are synthesized and administered to the subject. In further embodiments mutations in the oncogene are identified and peptides are selected to comprise the mutation in a T cell exposed position. In particular embodiments the copy number of the oncogene in the tumor tissue exceeds 5 fold that in normal tissue; in yet other embodiments copy number of the oncogene in the tumor tissue exceeds 10 fold that in normal tissue. In yet further embodiments, the amino acids in the MHC groove exposed positions of the selected peptides are changed to provide alternative peptides that change the predicted MHC binding affinity to a desired affinity. In some particular instances, the copy number of one or more of the bystander genes in the biopsy is also increased. In some embodiments the MHC binding is to an MHC I allele, in yet other instances the MHC binding is to a MHC II allele. In some embodiments the selected peptides are 9 or 10 amino acids in length; in yet other embodiments the selected peptides are from 13-20 amino acids long. In a further embodiment, the selected peptides may be from 8 to 30 amino acids long. In some embodiments the binding affinity of the peptide to the MHC allele is predicted to be is less than 20 nanomolar; in other embodiments it is less than 50 or 100 or 500 nanomolar.

In some embodiments of the present invention the subject from which the biopsy is obtained is suffering from cancer, which may be a cancer affecting the brain, liver, lung, breast, prostate, pancreas, genitourinary tract, gastrointestinal tract or may be a hematologic cancer, although these examples are not considered limiting. In some particular embodiments, the cancer of the brain is a glioblastoma, glioma, astrocytoma, meningioma, schwannoma, or may have arisen as a metastasis from another tissue.

The oncogene that is upregulated and increased in copy number may be any oncogene, but in particular embodiments is drawn from the list of oncogenes comprising: EGFR, PDGFA, ERRB2, MDM2, MYC, MYCN, or CDK4. Dysregulation of EGFR is a common occurrence in glioblastoma and bystander proteins encoded close to EGFR on chromosome 7 comprise SEC61G, VOPP, LANC2 and SEPT14. Thus, these are of particular interest as exemplar embodiments of the present invention. A number of T cell epitopes within these four bystander proteins are identified and the corresponding peptide, T cell exposed motifs and predicted MHC I and MHC II binding are identified. In some embodiments one or more peptides from one or more of the four bystander proteins to EGFR are comprised in a synthetic peptide array that is administered to the subject. In some particular embodiments the peptides are further distinguished by being more likely to be presented to T cells in vivo because of their higher probability of excision and processing by cathepsin endopeptidases enabling their presentation on MHC molecules. In yet other embodiments, the one or more synthetic peptides of the bystander proteins are co-administered with synthetic peptides derived from EGFR. In these particular embodiments, the peptides from EGFR encompass a T cell exposed motif that is tumor specific in that it exposes to the cognate T cell receptor an amino acid motif that is unique to the tumor and that is not found in normal EGFR. Such specificity may arise by mutation or by splice variant. In particular instances, certain common mutations of EGFR may be present in the T cell exposed motif. In yet other instances a mutation in EGFR may be unique to the individual subject. In yet other embodiments the tumor specific T cell exposed motif arises from a splice variant or deletion, such as the common variant EGFRvIII.

In preferred embodiments the peptides described above, that are selected from oncogenes and their bystander proteins based on the criteria described, are synthesized and incorporated into a vaccine which is applied to a subject. Because of the unique combination of peptides and the necessity to bind to the MHC alleles of the individual subject, such a vaccine may be designed specifically for the individual as a personal vaccine. The vaccine is prepared for administration, in some desired embodiments, by suspension in a pharmaceutically acceptable carrier which may in addition, in some embodiments, comprise an adjuvant. In some instances, such a vaccine is designed to be administered parenterally, whether intradermally or by other route selected by the clinician. In some cases, the intradermal vaccine may be administered by a microneedle array. In yet other embodiments a non parenteral route is preferred, which may include, but is not limited to oral delivery.

While the above embodiments refer to peptides as T cell stimulating epitopes, it will be well known to those skilled in the art that the one or more peptides may be encoded in a nucleic acid, either as a RNA or DNA or encoded in a gene delivery vector for application to the subject. In addition, in yet another embodiment, in lieu of administration of the peptide or encoding nucleic acid directly to the subject, these moieties may be contacted in vitro with antigen presenting cells drawn from the subject and the autologous cells later reinfused into the subject. In further embodiments of the invention, the peptides identified in the oncogene and bystander proteins may be applied in an in vitro assay, which is used to monitor the progress of the immune response of the subject. Such in vitro monitoring may be by implementation of an ELISPOT assay or other measurement of epitope specific T cell responses of the subject.

Accordingly, in some preferred embodiments, the present invention provides methods for treating cancer in a subject, comprising: designing a group of one or more T-cell stimulating peptides, or nucleic acids encoding T cell stimulating peptides, which have a desired predicted binding affinity for the MHC alleles of the subject, comprising the following steps: obtaining a biopsy of the subject's tumor; obtaining sequences for nucleic acids and proteins in the biopsy; comparing the copy number differential of genes encoding each protein between tumor and normal tissue; identifying proteins from the biopsy comprising an oncogene which is upregulated; identifying bystander proteins of the proteins that are transcribed; determining T cell exposed motifs in each of the bystander proteins; determining the predicted binding affinity to the subject's MHC alleles of peptides which comprises each of the T cell exposed motifs, or a subset thereof; selecting a group of one or more the peptides which have a desired predicted binding affinity for one or more of the subject's MHC alleles; synthesizing the group of one or more selected peptides, or nucleic acids encoding the selected peptides from the bystander proteins; and administering the selected peptides or nucleic acids to the subject.

In some preferred embodiments, the methods further comprise generating one or more alternative peptides not present in the tumor biopsy, wherein each alternative peptide comprises a T cell exposed motif identified in the bystander proteins, and in which the amino acids not within the T cell exposed motif are substituted to change the predicted binding affinity to the MHC alleles.

In some preferred embodiments, the oncogene is mutated in the tumor biopsy relative to the normal tissue.

In some preferred embodiments, the genes encoding the bystander proteins are present in increased copy number in the tumor biopsy. In some preferred embodiments, the copy number in the tumor biopsy of the oncogene is increased by more than five-fold over that in the normal tissue. In some preferred embodiments, the copy number in the tumor biopsy of the oncogene is increased by more than ten-fold over that in the normal tissue.

In some preferred embodiments, the MHC allele is an MHC I allele. In some preferred embodiments, the selected peptides are 9 or 10 amino acids long. In some preferred embodiments, the MHC allele is an MHC II allele. In some preferred embodiments, the selected peptides are 13 to 20 amino acids long. In some preferred embodiments, the selected peptides are from 8 to 30 amino acids long.

In some preferred embodiments, the predicted binding MHC affinity is to an MHC I allele carried by the subject. In some preferred embodiments, the predicted binding MHC affinity is to an MHC II allele carried by the subject. In some preferred embodiments, the desired predicted binding affinity of each selected peptide is less than 20 nanomolar. In some preferred embodiments, the desired predicted binding affinity of each selected peptide is less than 50 nanomolar. In some preferred embodiments, the desired predicted binding affinity of each selected peptide is less than 100 nanomolar. In some preferred embodiments, the desired predicted binding affinity of each selected peptide is less than 500 nanomolar.

In some preferred embodiments, the cancer with which the subject is afflicted with is selected from the group consisting of lung cancer, breast cancer, brain cancer, liver cancer, prostate cancer, pancreatic cancer, renal cancer, ovarian or uterine cancer, gastrointestinal tract cancer and a hematologic cancer. In some preferred embodiments, the brain cancer is selected from the group consisting of glioma, glioblastoma, meningioma, astrocytoma, medulloblastoma, schwannoma and a metastasis from an extracranial site.

In some preferred embodiments, the oncogene is selected from the group consisting of EGFR, PDGFA, ERRB2, MDM2, MYC, MYCN, and CDK4 and combinations thereof. In some preferred embodiments, the oncogene is encoded on chromosome 7. In some preferred embodiments, the oncogene is EGFR and bystander proteins are selected from the group consisting of SEC61G, VOPP1, LANC2, and SEPT14 and combinations thereof. In some preferred embodiments, the bystander protein is SEC61G and selected peptides are selected from the group consisting of SEQ ID NOs: 1-12 and 25-36 and combinations thereof. In some preferred embodiments, the bystander protein is VOPP1 and selected peptides are selected from the group consisting of SEQ ID NOs: 97-126 and 157-169 and combinations thereof. In some preferred embodiments, the bystander protein is LANC2 and selected peptides are selected from the group consisting of SEQ ID NOs: 206-256 and 308-370 and combinations thereof. In some preferred embodiments, the bystander protein is SEPT14 and selected peptides are selected from the group consisting of SEQ ID NOs: 457-487 and 546-574 and combinations thereof.

In some preferred embodiments, the peptides are excised by cathepsin S or cathepsin L.

In some preferred embodiments, the T cell exposed motif identified in the bystander proteins are selected from the group consisting of SEQ ID NOs: 13-24 and 37-48 and combinations thereof. In some preferred embodiments, the T cell exposed motif identified in the bystander proteins are selected from the group consisting of SEQ ID NOs: 127-156 and 170-182 and combinations thereof. In some preferred embodiments, the T cell exposed motif identified in the bystander proteins are selected from the group consisting of SEQ ID NOs: 257-307 and 371-433 and combinations thereof. In some preferred embodiments, the T cell exposed motif identified in the bystander proteins are selected from the group consisting of SEQ ID NOs: 488-545 and 575-603 and combinations thereof.

In some preferred embodiments, one or more of the selected peptides from the bystander protein is co-administered with a peptide comprising a T cell exposed motif of their adjacent oncogene. In some preferred embodiments, one or more of the peptides is co-administered with a peptide comprising a T cell exposed motif of EGFR. In some preferred embodiments, the T cell exposed motif of EGFR is selected from the group consisting of SEQ ID NOs: 604-708 and combinations thereof. In some preferred embodiments, one or more of the peptides is co-administered with a peptide comprising a T cell exposed motif of EGFR are selected from the group consisting of SEQ ID NOs: 717-734 and combinations thereof.

In some preferred embodiments, at least 2 peptides that bind to MHC I alleles or nucleic acids encoding the peptides that bind to MHC I alleles are selected for synthesis and/or coadministration to the subject. In some preferred embodiments, at least 5 peptides that bind to MHC I alleles or nucleic acids encoding the peptides that bind to MHC I alleles are selected for synthesis and/or coadministration to the subject. In some preferred embodiments, at least 10 peptides that bind to MHC I alleles or nucleic acids encoding the peptides that bind to MHC I alleles are selected for synthesis and/or coadministration to the subject. In some preferred embodiments, at least 15 peptides that bind to MHC I alleles or nucleic acids encoding the peptides that bind to MHC I alleles are selected for synthesis and/or coadministration to the subject. In some preferred embodiments, at least 20 peptides that bind to MHC I alleles or nucleic acids encoding the peptides that bind to MHC I alleles are selected for synthesis and/or coadministration to the subject. In some preferred embodiments, at least 2 peptides that bind to MHC II alleles or nucleic acids encoding the peptides that bind to MHC II alleles are selected for synthesis and/or coadministration to the subject. In some preferred embodiments, at least 5 peptides that bind to MHC II alleles or nucleic acids encoding the peptides that bind to MHC II alleles are selected for synthesis and/or coadministration to the subject. In some preferred embodiments, at least 10 peptides that bind to MHC II alleles or nucleic acids encoding the peptides that bind to MHC II alleles are selected for synthesis and/or coadministration to the subject. In some preferred embodiments, at least 15 peptides that bind to MHC II alleles or nucleic acids encoding the peptides that bind to MHC II alleles are selected for synthesis and/or coadministration to the subject. In some preferred embodiments, at least 20 peptides that bind to MHC II alleles or nucleic acids encoding the peptides that bind to MHC II alleles are selected for synthesis and/or coadministration to the subject. In some preferred embodiments, the peptides (ore nucleic acids encoding the peptides) selected for synthesis and/or coadministration to the subject comprise a combination of peptides that bind to MHC I alleles and MHC II alleles according to the foregoing ranges (e.g., at least 5 peptides that bind MHC I alleles and at least 5 peptides that bind MHC II alleles, and so on).

In some preferred embodiments, from 2 to 50 peptides that bind to MHC I alleles or nucleic acids encoding the peptides that bind to MHC I alleles are selected for synthesis and/or coadministration to the subject. In some preferred embodiments, from 5 to 50 peptides that bind to MHC I alleles or nucleic acids encoding the peptides that bind to MHC I alleles are selected for synthesis and/or coadministration to the subject. In some preferred embodiments, from 10 to 50 peptides that bind to MHC I alleles or nucleic acids encoding the peptides that bind to MHC I alleles are selected for synthesis and/or coadministration to the subject. In some preferred embodiments, from 15 to 50 peptides that bind to MHC I alleles or nucleic acids encoding the peptides that bind to MHC I alleles are selected for synthesis and/or coadministration to the subject. In some preferred embodiments, from 20 to 50 peptides that bind to MHC I alleles or nucleic acids encoding the peptides that bind to MHC I alleles are selected for synthesis and/or coadministration to the subject. In some preferred embodiments, from 2 to 50 peptides that bind to MHC II alleles or nucleic acids encoding the peptides that bind to MHC II alleles are selected for synthesis and/or coadministration to the subject. In some preferred embodiments, from 5 to 100 peptides that bind to MHC II alleles or nucleic acids encoding the peptides that bind to MHC II alleles are selected for synthesis and/or coadministration to the subject. In some preferred embodiments, from 10 to 50 peptides that bind to MHC II alleles or nucleic acids encoding the peptides that bind to MHC II alleles are selected for synthesis and/or coadministration to the subject. In some preferred embodiments, from 15 to 50 peptides that bind to MHC II alleles or nucleic acids encoding the peptides that bind to MHC II alleles are selected for synthesis and/or coadministration to the subject. In some preferred embodiments, from 20 to 50 peptides that bind to MHC II alleles or nucleic acids encoding the peptides that bind to MHC II alleles are selected for synthesis and/or coadministration to the subject. In some preferred embodiments, the peptides (ore nucleic acids encoding the peptides) selected for synthesis and/or coadministration to the subject comprise a combination of peptides that bind to MHC I alleles and MHC II alleles according to the foregoing ranges (e.g., from 5 to 50 peptides that bind MHC I alleles and from 5 to 50 peptides that bind MHC II alleles, and so on).

In some preferred embodiments, the group of one or more selected peptides is administered to a subject as a vaccine. In some preferred embodiments, the peptides in the group of one or more selected peptides are each encoded in nucleic acid which is administered to a subject as a vaccine. In some preferred embodiments, the nucleic acid is RNA. In some preferred embodiments, the nucleic acid is DNA. In some preferred embodiments, the nucleic acid is provided in a vector. In some preferred embodiments, the vaccine is administered in a pharmaceutically acceptable carrier. In some preferred embodiments, the vaccine also comprises an adjuvant.

In some preferred embodiments, the present invention provides a vaccine comprising one or more selected peptides identified as described above or a nucleic acid encoding one or more selected peptides identified as described above. In some preferred embodiments, the nucleic acid is RNA. In some preferred embodiments, the nucleic acid is DNA. In some preferred embodiments, the nucleic acid is provided in a vector. In some preferred embodiments, the vaccine is administered in a pharmaceutically acceptable carrier. In some preferred embodiments, the vaccine also comprises an adjuvant. In some preferred embodiments, the adjuvant and/or pharmaceutically acceptable carrier do not naturally occur with the peptide or nucleic acid. In some preferred embodiments, the adjuvant increases the immune response to the peptide and/or nucleic acid in the vaccine.

In some preferred embodiments, the present invention provides a vaccination regimen comprising administering a group of peptides, or nucleic acids encoding the same peptides, selected according to the methods as described above or a vaccine as a described above to a subject with cancer.

In some preferred embodiments, the present invention provides a vaccine comprising a peptide or nucleic acid as described above for use in treating a cancer or tumor. In some preferred embodiments, the cancer with which the subject is afflicted with is selected from the group consisting of lung cancer, breast cancer, brain cancer, liver cancer, prostate cancer, pancreatic cancer, renal cancer, ovarian or uterine cancer, gastrointestinal tract cancer and a hematologic cancer. In some preferred embodiments, the brain cancer is selected from the group consisting of glioma, glioblastoma, meningioma, astrocytoma, medulloblastoma, schwannoma and a metastasis from an extracranial site.

In some preferred embodiments, the vaccine is administered to a subject parenterally. In some preferred embodiments, the vaccine is administered to a subject intradermally. In some preferred embodiments, the vaccine is administered by microneedle array. In some preferred embodiments, the vaccine is administered to a subject non-parenterally. In some preferred embodiments, the vaccine is administered orally.

In some preferred embodiments, the present invention provides methods comprising administering a group of peptides, or nucleic acids encoding the same peptides, selected according to the methods as described above or a vaccine as described above in vitro to antigen presenting cells of the subject.

In some preferred embodiments, the present invention provides a diagnostic test (or kit for performing a diagnostic test) comprising a capture reagent(s) selected from the group consisting of one or more of the peptides identified by SEQ ID NO above. In some preferred embodiments, the test is applied to monitor the T cell responses of a subject affected by cancer.

### DESCRIPTION OF THE FIGURES

FIG. 1: Gene Track from the Integrated Genome Viewer showing a region of chromosome 7 in hg38 encoding EGFR. There are four other proteins encoded in the near vicinity of EGFR on chromosome 7. The unannotated transcripts are long non-coding RNAs.

FIG. 2: Shows the Lognormal distribution of exome data from tumor FPKM showing the effect of a log 10 transform.

FIG. 3: Histograms of log 10 FPKM data from a tumor and a normal exome dataset with different numbers of reads and fit with a SHASH distribution function.

FIG. 4: SHASH distribution transformed to a zero mean unit variance distribution. Line represents a normal distribution.

FIG. 5: Shows an example of copy number comparison between tumor and normal for a GBM patient (subject B) in which upregulated EGFR and coamplified SEC61G proteins are clearly observed compared to a comparison of tumor normal from a different GBM subject (Subject A) in which EGFR is not upregulated. Each datapoint represents the paired comparison of the tumor and normal copy number with the value being that of the normalized FPKM of one unique transcript (ENST) defined in GCRh38. In addition, in this graphic each point is colored based on the mRNA transcript enumeration in the tumor biopsy using the same normalization methodology (see scale on right side of EGFR upregulated subject).

FIG. 6: Annotated copy number comparison between tumor and normal in Subject B showing Sec61G along with EGFR transcripts.

FIG. 7: Subject C showing copy number comparison between tumor and normal by individual chromosome, showing EGFR and bystanders upregulated in chromosome 7.

FIG. 8: Epitope mapping of SEC61G. Background colors indicate extramembrane (yellow, transmembrane (green) and intramembrane (pink) domains. The X axis indicates the index position of sequential peptides with single amino acid displacement. The Y axis indicates predicted binding affinity of each peptide in standard deviation units for the protein. The red line shows the permuted average predicted MHC-IA and B (62 alleles) binding affinity of sequential 9-mer peptides with single amino acid displacement. The blue line shows the permuted average predicted MHC-II DRB allele (24 most common human alleles) binding affinity of sequential 15-mer peptides. Orange lines show the predicted probability of B-cell receptor binding for an amino acid centered in each sequential 9-mer peptide. Low numbers for MHC data represent high binding affinity, whereas low numbers equate to high B cell receptor contact probability. Ribbons (red: MHC-I, blue: MHC-II) indicate the 10% highest predicted MHC affinity binding. Orange ribbons indicate the top 25% predicted probability B-cell binding. Horizontal dotted lines demarcate the top 5% of binding affinity for the protein (red MHC I, blue MHC II).

FIG. 9: Epitope mapping of VOPP. Background colors indicate extramembrane (yellow, transmembrane (green) and intramembrane (pink) domains. The X axis indicates the index position of sequential peptides with single amino acid displacement. The Y axis indicates predicted binding affinity of each peptide in standard deviation units for the protein. The red line shows the permuted average predicted MHC-IA and B (62 alleles) binding affinity of sequential 9-mer peptides with single amino acid displacement. The blue line shows the permuted average predicted MHC-II DRB allele (24 most common human alleles) binding affinity of sequential 15-mer peptides. Orange lines show the predicted probability of B-cell receptor binding for an amino acid centered in each sequential 9-mer peptide. Low numbers for MHC data represent high binding affinity, whereas low numbers equate to high B cell receptor contact probability. Ribbons (red: MHC-I, blue: MHC-II) indicate the 10% highest predicted MHC affinity binding. Orange ribbons indicate the top 25% predicted probability B-cell binding. Horizontal dotted lines demarcate the top 5% of binding affinity for the protein (red MHC I, blue MHC II).

FIG. 10: Epitope mapping of LANC2. The X axis indicates the index position of sequential peptides with single amino acid displacement. The Y axis indicates predicted binding affinity of each peptide in standard deviation units for the protein. The red line shows the permuted average predicted MHC-IA and B (62 alleles) binding affinity of sequential 9-mer peptides with single amino acid displacement. The blue line shows the permuted average predicted MHC-II DRB allele (24 most common human alleles) binding affinity of sequential 15-mer peptides. Orange lines show the predicted probability of B-cell receptor binding for an amino acid centered in each sequential 9-mer peptide. Low numbers for MHC data represent high binding affinity, whereas low numbers equate to high B cell receptor contact probability. Ribbons (red: MHC-I, blue: MHC-II) indicate the 10% highest predicted MHC affinity binding. Orange ribbons indicate the top 25% predicted probability B-cell binding. Horizontal dotted lines demarcate the top 5% of binding affinity for the protein (red MHC I, blue MHC II).

FIG. 11: Epitope mapping of SEPT14. The X axis indicates the index position of sequential peptides with single amino acid displacement. The Y axis indicates predicted binding affinity of each peptide in standard deviation units for the protein. The red line shows the permuted average predicted MHC-IA and B (62 alleles) binding affinity of sequential 9-mer peptides with single amino acid displacement. The blue line shows the permuted average predicted MHC-II DRB allele (24 most common human alleles) binding affinity of sequential 15-mer peptides. Orange lines show the predicted probability of B-cell receptor binding for an amino acid centered in each sequential 9-mer peptide. Low numbers for MHC data represent high binding affinity, whereas low numbers equate to high B cell receptor contact probability. Ribbons (red: MHC-I, blue: MHC-II) indicate the 10% highest predicted MHC affinity binding. Orange ribbons indicate the top 25% predicted probability B-cell binding. Horizontal dotted lines demarcate the top 5% of binding affinity for the protein (red MHC I, blue MHC II).

FIG. 12. Gene Track from the Integrated Genome Viewer showing a region of chromosome 4 in hg38 encoding PDGFA. There are 2 other proteins encoded in the near vicinity of PDGFA on chromosome 4. The unannotated transcripts are long non-coding RNAs.

FIG. 13. Gene Track from the Integrated Genome Viewer showing a region of chromosome 17 in hg38 encoding ERBB2. There are seven other proteins encoded in the near vicinity of ERBB2 on chromosome 17. The unannotated transcripts are long non-coding RNAs.

FIG. 14. Gene Track from the Integrated Genome Viewer showing a region of chromosome 12 in hg38 encoding MDM2. There are four other proteins encoded in the near vicinity of MDM2 on chromosome 12. The unannotated transcripts are long non-coding RNAs.

FIG. 15. Gene Track from the Integrated Genome Viewer showing a region of chromosome 12 in hg38 encoding CDK4. There are four other proteins encoded in the near vicinity of CDK4 on chromosome 7. The unannotated transcripts are long non-coding RNAs.

FIG. 16. Gene Track from the Integrated Genome Viewer showing a region of chromosome 8 in hg38 encoding MYCR. There is one other proteins encoded in the near vicinity of MYC on chromosome 8. The unannotated transcripts are long non-coding RNAs.

FIG. 17. Gene Track from the Integrated Genome Viewer showing a region of chromosome 2 in hg38 encoding MYCN. There is one other proteins encoded in the near vicinity of MYCN on chromosome 2. The unannotated transcripts are long non-coding RNAs.

### DEFINITIONS

As used herein, the term “genome” refers to the genetic material (e.g., chromosomes) of an organism or a host cell.

As used herein, the term “proteome” refers to the entire set of proteins expressed by a genome, cell, tissue or organism. A “partial proteome” refers to a subset the entire set of proteins expressed by a genome, cell, tissue or organism. Examples of “partial proteomes” include, but are not limited to, transmembrane proteins, secreted proteins, and proteins with a membrane motif. Human proteome refers to all the proteins comprised in a human being. Multiple such sets of proteins have been sequenced and are accessible at the InterPro international repository (on the world wide web at ebi.ac.uk/interpro). Human proteome is also understood to include those proteins and antigens thereof which may be over-expressed in certain pathologies, or expressed in a different isoforms in certain pathologies. Hence, as used herein, tumor associated antigens are considered part of the human proteome. “Proteome” may also be used to describe a large compilation or collection of proteins, such as all the proteins in an immunoglobulin collection or a T cell receptor repertoire, or the proteins which comprise a collection such as the allergome, such that the collection is a proteome which may be subject to analysis. All the proteins in a bacteria or other microorganism are considered its proteome.

As used herein, the terms “protein,” “polypeptide,” and “peptide” refer to a molecule comprising amino acids joined via peptide bonds. In general “peptide” is used to refer to a sequence of 40 or less amino acids and “polypeptide” is used to refer to a sequence of greater than 40 amino acids.

As used herein, the term, “synthetic polypeptide,” “synthetic peptide” and “synthetic protein” refer to peptides, polypeptides, and proteins that are produced by a recombinant process (i.e., expression of exogenous nucleic acid encoding the peptide, polypeptide or protein in an organism, host cell, or cell-free system) or by chemical synthesis.

As used herein, the term “protein of interest” refers to a protein encoded by a nucleic acid of interest. It may be applied to any protein to which further analysis is applied or the properties of which are tested or examined. Similarly, as used herein, “target protein” may be used to describe a protein of interest that is subject to further analysis.

As used herein “peptidase” refers to an enzyme which cleaves a protein or peptide. The term peptidase may be used interchangeably with protease, proteinases, oligopeptidases, and proteolytic enzymes. Peptidases may be endopeptidases (endoproteases), or exopeptidases (exoproteases). The term peptidase would also include the proteasome which is a complex organelle containing different subunits each having a different type of characteristic scissile bond cleavage specificity. Similarly the term peptidase inhibitor may be used interchangeably with protease inhibitor or inhibitor of any of the other alternate terms for peptidase.

As used herein, the term “exopeptidase” refers to a peptidase that requires a free N-terminal amino group, C-terminal carboxyl group or both, and hydrolyses a bond not more than three residues from the terminus. The exopeptidases are further divided into aminopeptidases, carboxypeptidases, dipeptidyl-peptidases, peptidyl-dipeptidases, tripeptidyl-peptidases and dipeptidases.

As used herein, the term “endopeptidase” refers to a peptidase that hydrolyses internal, alpha-peptide bonds in a polypeptide chain, tending to act away from the N-terminus or C-terminus. Examples of endopeptidases are chymotrypsin, pepsin, papain and cathepsins. A very few endopeptidases act a fixed distance from one terminus of the substrate, an example being mitochondrial intermediate peptidase. Some endopeptidases act only on substrates smaller than proteins, and these are termed oligopeptidases. An example of an oligopeptidase is thimet oligopeptidase. Endopeptidases initiate the digestion of food proteins, generating new N- and C-termini that are substrates for the exopeptidases that complete the process. Endopeptidases also process proteins by limited proteolysis. Examples are the removal of signal peptides from secreted proteins (e.g. signal peptidase I) and the maturation of precursor proteins (e.g., enteropeptidase, furin). In the nomenclature of the Nomenclature Committee of the International Union of Biochemistry and Molecular Biology (NC-IUBMB) endopeptidases are allocated to sub-subclasses EC 3.4.21, EC 3.4.22, EC 3.4.23, EC 3.4.24 and EC 3.4.25 for serine-, cysteine-, aspartic-, metallo- and threonine-type endopeptidases, respectively. Endopeptidases of particular interest are the cathepsins, and especially cathepsin B, L and S known to be active in antigen presenting cells.

As used herein, the term “immunogen” refers to a molecule which stimulates a response from the adaptive immune system, which may include responses drawn from the group comprising an antibody response, a cytotoxic T cell response, a T helper response, and a T cell memory. An immunogen may stimulate an upregulation of the immune response with a resultant inflammatory response, or may result in down regulation or immunosuppression. Thus the T-cell response may be a T regulatory response. An immunogen also may stimulate a B-cell response and lead to an increase in antibody titer. Another term used herein to describe a molecule or combination of molecules which stimulate an immune response is “antigen”.

As used herein, the term “native” (or wild type) when used in reference to a protein refers to proteins encoded by the genome of a cell, tissue, or organism, other than one manipulated to produce synthetic proteins.

As used herein the term “epitope” refers to a peptide sequence which elicits an immune response, from either T cells or B cells or antibody

As used herein, the term “B-cell epitope” refers to a polypeptide sequence that is recognized and bound by a B-cell receptor. A B-cell epitope may be a linear peptide or may comprise several discontinuous sequences which together are folded to form a structural epitope. Such component sequences which together make up a B-cell epitope are referred to herein as B-cell epitope sequences. Hence, a B-cell epitope may comprise one or more B-cell epitope sequences. Hence, a B cell epitope may comprise one or more B-cell epitope sequences. A linear B-cell epitope may comprise as few as 2-4 amino acids or more amino acids.

As used herein, the term “predicted B-cell epitope” refers to a polypeptide sequence that is predicted to bind to a B-cell receptor by a computer program, for example, as described in PCT US2011/029192, PCT US2012/055038, US2014/014523, and PCT US2015/039969, each of which is incorporated herein by reference in its entirety, and in addition by Bepipred (Larsen, et al., Immunome Research 2:2, 2006) and others as referenced by Larsen et al (ibid) (Hopp T et al PNAS 78:3824-3828, 1981; Parker J et al, Biochem. 25:5425-5432, 1986). A predicted B-cell epitope may refer to the identification of B-cell epitope sequences forming part of a structural B-cell epitope or to a complete B-cell epitope.

As used herein, the term “T-cell epitope” refers to a polypeptide sequence which when bound to a major histocompatibility protein molecule provides a configuration recognized by a T-cell receptor. Typically, T-cell epitopes are presented bound to a MHC molecule on the surface of an antigen-presenting cell.

As used herein, the term “predicted T-cell epitope” refers to a polypeptide sequence that is predicted to bind to a major histocompatibility protein molecule by the neural network algorithms described herein, by other computerized methods, or as determined experimentally. As used herein, the term “major histocompatibility complex (MHC)” refers to the MHC Class I and MHC Class II genes and the proteins encoded thereby. Molecules of the MHC bind small peptides and present them on the surface of cells for recognition by T-cell receptor-bearing T-cells. The MHC is both polygenic (there are several MHC class I and MHC class II genes) and polyallelic or polymorphic (there are multiple alleles of each gene). The terms MHC-I, MHC-II, MHC-1 and MHC-2 are variously used herein to indicate these classes of molecules. Included are both classical and nonclassical MHC molecules. An MHC molecule is made up of multiple chains (alpha and beta chains) which associate to form a molecule. The MHC molecule contains a cleft or groove which forms a binding site for peptides. Peptides bound in the cleft or groove may then be presented to T-cell receptors. The term “MHC binding region” refers to the groove region of the MHC molecule where peptide binding occurs.

As used herein, a “MHC II binding groove” refers to the structure of an MHC molecule that binds to a peptide. The peptide that binds to the MHC II binding groove may be from about 11 amino acids to about 23 amino acids in length, but typically comprises a 15-mer. The amino acid positions in the peptide that binds to the groove are numbered based on a central core of 9 amino acids numbered 1-9, and positions outside the 9 amino acid core numbered as negative (N terminal) or positive (C terminal). Hence, in a 15mer the amino acid binding positions are numbered from −3 to +3 or as follows: −3, −2, −1, 1, 2, 3, 4, 5, 6, 7, 8, 9, +1, +2, +3.

As used herein, the term “haplotype” refers to the HLA alleles found on one chromosome and the proteins encoded thereby. Haplotype may also refer to the allele present at any one locus within the MHC. Each class of MHC-Is represented by several loci: e.g., HLA-A (Human Leukocyte Antigen-A), HLA-B, HLA-C, HLA-E, HLA-F, HLA-G, HLA-H, HLA-J, HLA-K, HLA-L, HLA-P and HLA-V for class I and HLA-DRA, HLA-DRB1-9, HLA-, HLA-DQA1, HLA-DQB1, HLA-DPA1, HLA-DPB1, HLA-DMA, HLA-DMB, HLA-DOA, and HLA-DOB for class II. The terms “HLA allele” and “MHC allele” are used interchangeably herein. HLA alleles are listed at hla.alleles.org/nomenclature/naming.html, which is incorporated herein by reference.

The MHCs exhibit extreme polymorphism: within the human population there are, at each genetic locus, a great number of haplotypes comprising distinct alleles—the IMGT/HLA database release (February 2010) lists 948 class I and 633 class II molecules, many of which are represented at high frequency (>1%). MHC alleles may differ by as many as 30-aa substitutions. Different polymorphic MHC alleles, of both class I and class II, have different peptide specificities: each allele encodes proteins that bind peptides exhibiting particular sequence patterns.

The naming of new HLA genes and allele sequences and their quality control is the responsibility of the WHO Nomenclature Committee for Factors of the HLA System, which first met in 1968, and laid down the criteria for successive meetings. This committee meets regularly to discuss issues of nomenclature and has published 19 major reports documenting firstly the HLA antigens and more recently the genes and alleles. The standardization of HLA antigenic specifications has been controlled by the exchange of typing reagents and cells in the International Histocompatibility Workshops. The IMGT/HLA Database collects both new and confirmatory sequences, which are then expertly analyzed and curated before been named by the Nomenclature Committee. The resulting sequences are then included in the tools and files made available from both the IMGT/HLA Database and at hla.alleles.org.

Each HLA allele name has a unique number corresponding to up to four sets of digits separated by colons. See e.g., hla.alleles.org/nomenclature/naming.html which provides a description of standard HLA nomenclature and Marsh et al., Nomenclature for Factors of the HLA System, 2010 Tissue Antigens 2010 75:291-455. HLA-DRB1*13:01 and HLA-DRB1*13:01:01:02 are examples of standard HLA nomenclature. The length of the allele designation is dependent on the sequence of the allele and that of its nearest relative. All alleles receive at least a four digit name, which corresponds to the first two sets of digits, longer names are only assigned when necessary.

The digits before the first colon describe the type, which often corresponds to the serological antigen carried by an allele, The next set of digits are used to list the subtypes, numbers being assigned in the order in which DNA sequences have been determined. Alleles whose numbers differ in the two sets of digits must differ in one or more nucleotide substitutions that change the amino acid sequence of the encoded protein. Alleles that differ only by synonymous nucleotide substitutions (also called silent or non-coding substitutions) within the coding sequence are distinguished by the use of the third set of digits. Alleles that only differ by sequence polymorphisms in the introns or in the 5′ or 3′ untranslated regions that flank the exons and introns are distinguished by the use of the fourth set of digits. In addition to the unique allele number there are additional optional suffixes that may be added to an allele to indicate its expression status. Alleles that have been shown not to be expressed, ‘Null’ alleles have been given the suffix ‘N’. Those alleles which have been shown to be alternatively expressed may have the suffix ‘L’, ‘S’, ‘C’, ‘A’ or ‘Q’. The suffix ‘L’ is used to indicate an allele which has been shown to have ‘Low’ cell surface expression when compared to normal levels. The ‘S’ suffix is used to denote an allele specifying a protein which is expressed as a soluble ‘Secreted’ molecule but is not present on the cell surface. A ‘C’ suffix to indicate an allele product which is present in the ‘Cytoplasm’ but not on the cell surface. An ‘A’ suffix to indicate ‘Aberrant’ expression where there is some doubt as to whether a protein is expressed. A ‘Q’ suffix when the expression of an allele is ‘Questionable’ given that the mutation seen in the allele has previously been shown to affect normal expression levels.

In some instances, the HLA designations used herein may differ from the standard HLA nomenclature just described due to limitations in entering characters in the databases described herein. As an example, DRB1_0104, DRB1*0104, and DRB1-0104 are equivalent to the standard nomenclature of DRB1*01:04. In most instances, the asterisk is replaced with an underscore or dash and the semicolon between the two digit sets is omitted.

As used herein, the term “polypeptide sequence that binds to at least one major histocompatibility complex (MHC) binding region” refers to a polypeptide sequence that is recognized and bound by one or more particular MHC binding regions as predicted by the neural network algorithms described herein or as determined experimentally.

As used herein the terms “canonical” and “non-canonical” are used to refer to the orientation of an amino acid sequence. Canonical refers to an amino acid sequence presented or read in the N terminal to C terminal order; non-canonical is used to describe an amino acid sequence presented in the inverted or C terminal to N terminal order.

As used herein, the term “transmembrane protein” refers to proteins that span a biological membrane. There are two basic types of transmembrane proteins. Alpha-helical proteins are present in the inner membranes of bacterial cells or the plasma membrane of eukaryotes, and sometimes in the outer membranes. Beta-barrel proteins are found only in outer membranes of Gram-negative bacteria, cell wall of Gram-positive bacteria, and outer membranes of mitochondria and chloroplasts.

As used herein, the term “affinity” refers to a measure of the strength of binding between two members of a binding pair, for example, an antibody and an epitope and an epitope and a MHC-I or II haplotype. K_{d} is the dissociation constant and has units of molarity. The affinity constant is the inverse of the dissociation constant. An affinity constant is sometimes used as a generic term to describe this chemical entity. It is a direct measure of the energy of binding. The natural logarithm of K is linearly related to the Gibbs free energy of binding through the equation ΔG₀=−RT LN(K) where R=gas constant and temperature is in degrees Kelvin. Affinity may be determined experimentally, for example by surface plasmon resonance (SPR) using commercially available Biacore SPR units (GE Healthcare) or in silico by methods such as those described herein in detail. Affinity may also be expressed as the ic50 or inhibitory concentration 50, that concentration at which 50% of the peptide is displaced. Likewise ln(ic50) refers to the natural log of the ic50.

The term “K_{off}”, as used herein, is intended to refer to the off rate constant, for example, for dissociation of an antibody from the antibody/antigen complex, or for dissociation of an epitope from an MHC haplotype.

The term “K_{d}”, as used herein, is intended to refer to the dissociation constant (the reciprocal of the affinity constant “K_{d}”), for example, for a particular antibody-antigen interaction or interaction between an epitope and an MHC haplotype.

As used herein, the terms “strong binder” and “strong binding” and “High binder” and “high binding” or “high affinity” refer to a binding pair or describe a binding pair that have an affinity of greater than 2×10⁷ M⁻¹ (equivalent to a dissociation constant of 50 nM Kd)

As used herein, the term “moderate binder” and “moderate binding” and “moderate affinity” refer to a binding pair or describe a binding pair that have an affinity of from 2×10⁷ M⁻¹ to 2×10⁶ M⁻¹.

As used herein, the terms “weak binder” and “weak binding” and “low affinity” refer to a binding pair or describe a binding pair that have an affinity of less than 2×10⁶ M⁻¹ (equivalent to a dissociation constant of 500 nM Kd)

Binding affinity may also be expressed by the standard deviation from the mean binding found in the peptides making up a protein. Hence a binding affinity may be expressed as “−1σ” or <−1σ, where this refers to a binding affinity of 1 or more standard deviations below the mean. A common mathematical transformation used in statistical analysis is a process called standardization wherein the distribution is transformed from its standard units to standard deviation units where the distribution has a mean of zero and a variance (and standard deviation) of 1. Because each protein comprises unique distributions for the different MHC alleles standardization of the affinity data to zero mean and unit variance provides a numerical scale where different alleles and different proteins can be compared. Analysis of a wide range of experimental results suggest that a criterion of standard deviation units can be used to discriminate between potential immunological responses and non-responses. An affinity of 1 standard deviation below the mean was found to be a useful threshold in this regard and thus approximately 15% (16.2% to be exact) of the peptides found in any protein will fall into this category.

The terms “specific binding” or “specifically binding” when used in reference to the interaction of an antibody and a protein or peptide or an epitope and an WIC haplotype means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabeled A) in a reaction containing labeled “A” and the antibody will reduce the amount of labeled A bound to the antibody.

As used herein, the term “antigen binding protein” refers to proteins that bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries. Various procedures known in the art are used for the production of polyclonal antibodies. For the production of antibody, various host animals can be immunized by injection with the peptide corresponding to the desired epitope including but not limited to rabbits, mice, rats, sheep, goats, etc.

“Adjuvant” as used herein encompasses various adjuvants that are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, squalene, squalene emulsions, liposomes, imiquimod, keyhole limpet hemocyanins, dinitrophenol, and potentially useful human adjuvants such as BCG (Bacille Calmette-Guerin) and Corynebacterium parvum. In other embodiments a cytokine may be co-administered, including but not limited to interferon gamma or stimulators thereof, interleukin 12, or granulocyte stimulating factor. In other embodiments the peptides or their encoding nucleic acids may be co-administered with a local inflammatory agent, either chemical or physical. Examples include, but are not limited to, heat, infrared light, proinflammatory drugs, including but not limited to imiquimod.

As used herein “immunoglobulin” means the distinct antibody molecule secreted by a clonal line of B cells; hence when the term “100 immunoglobulins” is used it conveys the distinct products of 100 different B-cell clones and their lineages.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “support vector machine” refers to a set of related supervised learning methods used for classification and regression. Given a set of training examples, each marked as belonging to one of two categories, an SVM training algorithm builds a model that predicts whether a new example falls into one category or the other.

As used herein, the term “classifier” when used in relation to statistical processes refers to processes such as neural nets and support vector machines.

As used herein “neural net”, which is used interchangeably with “neural network” and sometimes abbreviated as NN, refers to various configurations of classifiers used in machine learning, including multilayered perceptrons with one or more hidden layer, support vector machines and dynamic Bayesian networks. These methods share in common the ability to be trained, the quality of their training evaluated, and their ability to make either categorical classifications of non-numeric data or to generate equations for predictions of continuous numbers in a regression mode. Perceptron as used herein is a classifier which maps its input x to an output value which is a function of x, or a graphical representation thereof.

As used herein, the term “principal component analysis”, or as abbreviated “PCA”, refers to a mathematical process which reduces the dimensionality of a set of data (Wold, S., Sjorstrom, M., and Eriksson, L., Chemometrics and Intelligent Laboratory Systems 2001. 58: 109-130; Multivariate and Megavariate Data Analysis Basic Principles and Applications (Parts I & II) by L. Eriksson, E. Johansson, N. Kettaneh-Wold, and J. Trygg, 2006 2^{nd} Edit. Umetrics Academy). Derivation of principal components is a linear transformation that locates directions of maximum variance in the original input data, and rotates the data along these axes. For n original variables, n principal components are formed as follows: The first principal component is the linear combination of the standardized original variables that has the greatest possible variance. Each subsequent principal component is the linear combination of the standardized original variables that has the greatest possible variance and is uncorrelated with all previously defined components. Further, the principal components are scale-independent in that they can be developed from different types of measurements. The application of PCA generates numerical coefficients (descriptors). The coefficients are effectively proxy variables whose numerical values are seen to be related to underlying physical properties of the molecules. A description of the application of PCA to generate descriptors of amino acids and by combination thereof peptides is provided in PCT US2011/029192 incorporated herein by reference in its entirety. Unlike neural nets PCA do not have any predictive capability. PCA is deductive not inductive.

As used herein, the term “vector” when used in relation to a computer algorithm or the present invention, refers to the mathematical properties of the amino acid sequence.

As used herein, the term “vector,” when used in relation to recombinant DNA technology, refers to any genetic element, such as a plasmid, phage, transposon, cosmid, chromosome, retrovirus, virion, etc., which is capable of replication when associated with the proper control elements and which can transfer gene sequences between cells. Thus, the term includes cloning and expression vehicles, as well as viral vectors. “Viral vector” as used herein includes but is not limited to adenoviral vectors, adeno-associated viral vectors, lentiviral vectors, retroviral vectors, poliovirus vectors, measles virus vectors, flavivirus vectors, poxvirus vectors, and other viral vectors which may be used to deliver a peptide or nucleic acid sequence to a host cell.

As used herein, the term “host cell” refers to any eukaryotic cell (e.g., mammalian cells, avian cells, amphibian cells, plant cells, fish cells, insect cells, yeast cells), and bacteria cells, and the like, whether located in vitro or in vivo (e.g., in a transgenic organism).

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acids are nucleic acids present in a form or setting that is different from that in which they are found in nature. In contrast, non-isolated nucleic acids are nucleic acids such as DNA and RNA that are found in the state in which they exist in nature.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

A “subject” is an animal such as vertebrate, preferably a mammal such as a human, a bird, or a fish. Mammals are understood to include, but are not limited to, murines, simians, humans, bovines, ovines, cervids, equines, porcines, canines, felines etc.). In some instances herein “subject” refers to a human patient who may be afflicted with cancer.

An “effective amount” is an amount sufficient to effect beneficial or desired results. An effective amount can be administered in one or more administrations,

As used herein, the term “purified” or “to purify” refers to the removal of undesired components from a sample. As used herein, the term “substantially purified” refers to molecules, either nucleic or amino acid sequences, that are removed from their natural environment, isolated or separated, and are at least 60% free, preferably 75% free, and most preferably 90% free from other components with which they are naturally associated. An “isolated polynucleotide” is therefore a substantially purified polynucleotide.

As used herein “Complementarity Determining Regions” (CDRs) are those parts of the immunoglobulin variable chains which determine how these molecules bind to their specific antigen. Each immunoglobulin variable region typically comprises three CDRs and these are the most highly variable regions of the molecule. T cell receptors also comprise similar CDRs and the term CDR may be applied to T cell receptors.

As used herein, the term “motif” refers to a characteristic sequence of amino acids forming a distinctive pattern.

The term “Groove Exposed Motif” (GEM) as used herein refers to a subset of amino acids within a peptide that binds to an MHC molecule; the GEM comprises those amino acids which are turned inward towards the groove formed by the MHC molecule and which play a significant role in determining the binding affinity. In the case of human MHC-I the GEM amino acids are typically (1,2,3,9). In the case of MHC-II molecules two formats of GEM are most common comprising amino acids (−3,2,−1,1,4,6,9,+1,+2,+3) and (−3,2,1,2,4,6,9,+1,+2,+3) based on a 15-mer peptide with a central core of 9 amino acids numbered 1-9 and positions outside the core numbered as negative (N terminal) or positive (C terminal).

“Immunoglobulin germline” is used herein to refer to the variable region sequences encoded in the inherited germline genes and which have not yet undergone any somatic hypermutation. Each individual carries and expresses multiple copies of germline genes for the variable regions of heavy and light chains. These undergo somatic hypermutation during affinity maturation. Information on the germline sequences of immunoglobulins is collated and referenced on the world wide web at imgt.org [4]. “Germline family” as used herein refers to the 7 main gene groups, catalogued at IMGT, which share similarity in their sequences and which are further subdivided into subfamilies.

“Affinity maturation” is the molecular evolution that occurs during somatic hypermutation during which unique variable region sequences generated that are the best at targeting and neutralizing and antigen become clonally expanded and dominate the responding cell populations.

“Germline motif” as used herein describes the amino acid subsets that are found in germline immunoglobulins. Germline motifs comprise both GEM and TCEM motifs found in the variable regions of immunoglobulins which have not yet undergone somatic hypermutation.

“Immunopathology” when used herein describes an abnormality of the immune system. An immunopathology may affect B-cells and their lineage causing qualitative or quantitative changes in the production of immunoglobulins. Immunopathologies may alternatively affect T-cells and result in abnormal T-cell responses. Immunopathologies may also affect the antigen presenting cells. Immunopathologies may be the result of neoplasias of the cells of the immune system. Immunopathology is also used to describe diseases mediated by the immune system such as autoimmune diseases. Illustrative examples of immunopathologies include, but are not limited to, B-cell lymphoma, T-cell lymphomas, Systemic Lupus Erythematosus (SLE), allergies, hypersensitivities, immunodeficiency syndromes, radiation exposure or chronic fatigue syndrome.

“pMHC” Is used to describe a complex of a peptide bound to an MHC molecule. In many instances a peptide bound to an MHC-I will be a 9-mer or 10-mer however other sizes of 7-11 amino acids may be thus bound. Similarly MHC-II molecules may form pMHC complexes with peptides of 15 amino acids or with peptides of other sizes from 11-23 amino acids. The term pMHC is thus understood to include any short peptide bound to a corresponding MHC.

“T-cell exposed motif” (also where abbreviated TCEM), as used herein, refers to the sub set of amino acids in a peptide bound in a MHC molecule which are directed outwards and exposed to a T-cell binding to the pMHC complex. A T-cell binds to a complex molecular space-shape made up of the outer surface MHC of the particular HLA allele and the exposed amino acids of the peptide bound within the MHC. Hence any T-cell recognizes a space shape or receptor which is specific to the combination of HLA and peptide. The amino acids which comprise the TCEM in an MHC-I binding peptide typically comprise positions 4, 5, 6, 7, 8 of a 9-mer. The amino acids which comprise the TCEM in an MHC-II binding peptide typically comprise 2, 3, 5, 7, 8 or −1, 3, 5, 7, 8 based on a 15-mer peptide with a central core of 9 amino acids numbered 1-9 and positions outside the core numbered as negative (N terminal) or positive (C terminal). As indicated under pMHC, the peptide bound to a MHC may be of other lengths and thus the numbering system here is considered a non-exclusive example of the instances of 9-mer and 15 mer peptides.

As used herein “histotope” refers to the outward facing surface of the MHC molecules which surrounds the T cell exposed motif and in combination with the T cell exposed motif serves as the binding surface for the T cell receptor.

As used herein the T cell receptor refers to the molecules exposed on the surface of a T cell which engage the histotope of the MHC and the T cell exposed motif of a peptide bound in the MHC. The T cell receptor comprises two protein chains, known as the alpha and beta chain in 95% of human T cells and as the delta and gamma chains in the remaining 5% of human T cells. Each chain comprises a variable region and a constant region. Each variable region comprises three complementarity determining regions or CDRs.

“Regulatory T-cell” or “Treg” as used herein, refers to a T-cell which has an immunosuppressive or down-regulatory function. Regulatory T-cells were formerly known as suppressor T-cells. Regulatory T-cells come in many forms but typically are characterized by expression CD4+, CD25, and Foxp3. Tregs are involved in shutting down immune responses after they have successfully eliminated invading organisms, and also in preventing immune responses to self-antigens or autoimmunity.

“uTOPE™ analysis” as used herein refers to the computer assisted processes for predicting binding of peptides to MHC and predicting cathepsin cleavage, described in PCT US2011/029192, PCT US2012/055038, US2014/014523, PCT US2015/039969, PCT US2017/021781, US Publ. No. 20130330335, US Publ. No. 20160132631, US Publ. No. 20170039314, US Publ. No 20170161430, US Publ. No. 20190070255, PCT US2020/037206, U.S. Pat. Nos. 10,706,955 and 10,755,801, each of which is incorporated by reference herein in its entirety.

“Isoform” as used herein refers to different forms of a protein which differ in a small number of amino acids. The isoform may be a full length protein (i.e., by reference to a reference wild-type protein or isoform) or a modified form of a partial protein, i.e., be shorter in length than a reference wild-type protein or isoform.

“Immunostimulation” as used herein refers to the signaling that leads to activation of an immune response, whether the immune response is characterized by a recruitment of cells or the release of cytokines which lead to suppression of the immune response. Thus, immunostimulation refers to both upregulation or down regulation.

“Up-regulation” as used herein refers to an immunostimulation which leads to cytokine release and cell recruitment tending to eliminate a non self or exogenous epitope. Such responses include recruitment of T cells, including effectors such as cytotoxic T cells, and inflammation. In an adverse reaction upregulation may be directed to a self-epitope.

“Down regulation” as used herein refers to an immunostimulation which leads to cytokine release that tends to dampen or eliminate a cell response. In some instances such elimination may include apoptosis of the responding T cells.

“Frequency class” or “frequency classification” as used herein is used to describe logarithmic based bins or subsets of amino acid motifs or cells. When applied to the counts of TCEM motifs found in a given dataset of peptides a logarithmic (log base 2) frequency categorization scheme was developed to describe the distribution of motifs in a dataset. As the cellular interactions between T-cells and antigen presenting cells displaying the motifs in MHC molecules on their surfaces are the ultimate result of the molecular interactions, using a log base 2 system implies that each adjacent frequency class would double or halve the cellular interactions with that motif. Thus, using such a frequency categorization scheme makes it possible to characterize subtle differences in motif usage as well as providing a comprehensible way of visualizing the cellular interaction dynamics with the different motifs. Hence a Frequency Class 2, or FC 2 means 1 in 4, a Frequency class 10 or FC 10 means 1 in 2¹⁰ or 1 in 1024. In other embodiments the frequency classification of the TCEM motif in the reference dataset is described by the quantile score of the TCEM in the reference dataset. Quantile scores are used, but is not limited to, applications where the reference dataset is the human proteome or a microbial proteome. “Frequency class” or “frequency classification” may also be applied to cellular clonotypic frequency where it refers to subgroups or bins defined by logarithmic based groupings, whether log base 2 or another selected log base.

A “rare TCEM” as used herein is one which is completely missing in the human proteome or present in up to only five instances in the human proteome.

“Clonotype” as used herein refers to the cell lineage arising from one unique cell. In the particular case of a B cell clonotype it refers to a clonal population of B cells that produces a unique sequence of IGV. The number of B cells that express that sequence varies from singletons to thousands in the repertoire of an individual. In the case of a T cell it refers to a cell lineage which expresses a particular TCR. A clonotype of cancer cells all arise from one cell and carry a particular mutation or mutations or the derivates thereof. The above are examples of clonotypes of cells and should not be considered limiting.

As used herein “epitope mimic” or “TCEM mimic” is used to describe a peptide which has an identical or overlapping TCEM, but may have a different GEM. Such a mimic occurring in one protein may induce an immune response directed towards another protein which carries the same TCEM motif. This may give rise to autoimmunity or inappropriate responses to the second protein.

“Cytokine” as used herein refers to a protein which is active in cell signaling and may include, among other examples, chemokines, interferons, interleukins, lymphokines, granulocyte colony-stimulating factor tumor necrosis factor and programmed death proteins.

As used herein “oncoprotein” means a protein encoded by an oncogene which can cause the transformation of a cell into a tumor cell if introduced into it. Examples of oncoproteins include but are not limited to the early proteins of papillomaviruses, polyomaviruses, adenoviruses and herpesviruses, however oncoproteins are not necessarily of viral origin.

“MHC subunit chain” as used herein refers to the alpha and beta subunits of MHC molecules. A MHC II molecule is made up of an alpha chain which is constant among each of the DR, DP, and DQ variants and a beta chain which varies by allele. The MHC I molecule is made up of a constant beta macroglobulin and a variable MHC A, B or C chain.

“Immunoglobulinome” as used herein refers to the total complement of immunoglobulins produced and carried by any one subject.

As used herein the term “repertoire’ is used to describe a collection of molecules or cells making up a functional unit or whole. Thus, as one non limiting example, the entirely of the B cells or T cells in a subject comprise its repertoire of B cells or T cells. The entirety of all immunoglobulins expressed by the B cells are its immunoglobulinome or the repertoire of immunoglobulins. A collection of proteins or cell clonotypes which make up a tissue sample, an individual subject or a microorganism may be referred to as a repertoire.

As used herein “mutated amino acid” refers to the appearance of an amino acid in a protein that is the result of a nucleotide change, a missense mutation, or an insertion or deletion or fusion.

“Splice variant” as used herein refers to different proteins that are expressed from one gene as the result of inclusion or exclusion of particular exons of a gene in the final, processed messenger RNA produced from that gene or that is the result of cutting and re-annealing of RNA or DNA.

“TRAV” as used herein refers to the T cell receptor alpha variable region family or allele subgroups and “TRBV” refers to T cell receptor beta variable region family or allele subgroups as described in IMGT (on the world wide web at imgt.org/IMGTrepertoire/Proteins/index.php#C and imgt.org/IMGTrepertoire/Proteins/taballeles/human/TRA/TRAV/Hu_TRAVall.html).TRAV comprises at least 41 subgroups, with some having sub-subgroups. TRBV comprises at least 30 subgroups. Most combinations of alpha and beta variable region subgroups are encountered. “hTRAV” refers to human TRAV.

As used here in a “receptor bearing cell” is any cell which carries a ligand binding recognition motif on its surface. In some particular instances a receptor bearing cell is a B cell and its surface receptor comprises an immunoglobulin variable region, the immunoglobulin variable region comprising both heavy and light chains which make up the receptor. In other particular instances a receptor bearing cell may be a T cell which bears a receptor made up of both alpha and beta chains or both delta and gamma chains. Other examples of a receptor bearing cell include cells which carry other ligands such as, in one particular non limiting example, a programmed death protein of which there are multiple isoforms.

As used herein the term “bin” refers to a quantitative grouping and a “logarithmic bin” is used to describe a grouping according to the logarithm of the quantity.

As used herein “immunotherapy intervention” is used to describe any deliberate modification of the immune system including but not limited to through the administration of therapeutic drugs or biopharmaceuticals, radiation, T cell therapy, application of engineered T cells, which may include T cells linked to cytotoxic, chemotherapeutic or radiosensitive moieties, checkpoint inhibitor administration, cytokine or recombinant cytokine or cytokine enhancer, including but not limited to a IL-15 agonist, microbiome manipulation, vaccination, B or T cell depletion or ablation, or surgical intervention to remove any immune related tissues.

As used herein “immunomodulatory intervention” refers to any medical or nutritional treatment or prophylaxis administered with the intent of changing the immune response or the balance of immune responsive cells. Such an intervention may be delivered parenterally or orally or via inhalation. Such intervention may include, but is not limited to, a vaccine including both prophylactic and therapeutic vaccines, a biopharmaceutical, which may be from the group comprising an immunoglobulin or part thereof, a T cell stimulator, checkpoint inhibitor, or suppressor, an adjuvant, a cytokine, a cytotoxin, receptor binder, an enhancer of NK (natural killer) cells, an interleukin including but not limited to variants of IL15, superagonists, and a nutritional or dietary supplement. The intervention may also include radiation or chemotherapy to ablate a target group of cells. The impact on the immune response may be to stimulate or to down regulate.

“Checkpoint inhibitor” or “checkpoint blockade” as used herein refers to a type of drug that blocks certain proteins made by some types of immune system cells, such as T cells, and some cancer cells. These proteins help keep immune responses in check and can keep T cells from killing cancer cells. When these proteins are blocked, the “brakes” on the immune system are released and T cells are able to kill cancer cells better. Examples of checkpoint proteins found on T cells or cancer cells include, but are not limited to, PD-1/PD-L1 and CTLA-4/B7-1/B7-2.

As used herein the “cluster of differentiation” proteins refers to cell surface molecules providing targets for immunophenotyping of cells. The cluster of differentiation is also known as cluster of designation or classification determinant and may be abbreviated as CD. Examples of CD proteins include those listed on the world wide web at uniprot.org/docs/cdlist.

As used herein “microbiome” refers to the constellation of commensal microorganisms found within the human or other host body, inhabiting sites such as the gastrointestine, skin the urogenital tract, the oral cavity, the upper respiratory tract. While most frequently referring to bacteria, the microbiome also may include the viruses in these sites, referred to as the “virome”, or commensal fungi.

As used herein “tumor associated mutations” refers to all nucleotide or amino acid mutations detected in a tumor. In some cases the tumor associated mutations are commonly found within many patients with a particular tumor type. In other cases tumor associated mutations may be unique to a specific patient. In other instances different patients may carry different tumor associated mutations are in the same protein.

“Pattern” as used herein means a characteristic or consistent distribution of data points.

As used herein a “frequency pattern” is a data set that displays the frequency of TCEMs in a repertoire of proteins from a proteome associated with an individual subject as compared to the frequency of those TCEMs in a reference database. Particular TCEMs, or groups of TCEMs, within the subject's repertoire may occur at the same, lower or higher frequencies than the corresponding TCEMs in the reference database. The frequency pattern allows identification and categorization of unique TCEMs and/or patterns of TCEMs (i.e., unique features of unique TCEM features). The term “frequency pattern” as used herein is also used to describe the distribution of cellular clonotypes within a repertoire of cells from an individual subject, as compared to the frequency of the cellular clonotypes in a reference database. Particular clonotypes, or groups of clonotypes, within the subject's repertoire may occur at the same, lower or higher frequencies than the corresponding cellular clonotypes in the reference database. The frequency pattern allows identification and categorization of unique patterns of clonotypes. In some embodiments, a “frequency class” or “frequency classification” is assigned to a TCEM motif or to a cellular clonotype based on its frequency as described elsewhere herein.

As used herein “clonotype” is a line of cells derived from a committed or fully differentiated progenitor. In the case of T cells and somatic cells other than B cells, a clonotype of cells has a common genotype, i.e. comprises a common nucleotide sequence. Clonotypes with different nucleotide sequences may express a protein of identical amino acid sequence as a result of different codon utilization. Hence multiple genotypes may lead to a shared phenotype among such clonotypes. In B cells, somatic mutation results in a differentiated cell line comprising a nucleotide sequence that expresses antibodies of one isotype and variable region sequence; this is a B cell clonotype.

As used herein “clonotypic diversity” refers to the distribution of the total number of cells in a repertoire among all unique clonotypes in a repertoire. Hence, if a repertoire has 1 million cells, but these comprise 400,000 of clonotype 1 and 600,000 of clonotype 2, the repertoire has a low clonotypic diversity. If the 1 million cells are distributed as 10 each of 100,000 unique clonotypes the repertoire has a high clonotypic diversity.

As used herein “many to one” describes a relationship in which one protein or peptide sequence is encoded be many different synonymous nucleotide sequences.

As used herein “presentome” refers to the peptides bound in MHC and presented on the surface of antigen presented cells. Mass spectroscopy detects some but not all peptides which are part of the presentome.

“Neoantigen” as used herein refers to a novel epitope motif or antigen created as the result of introduction of a mutation into an amino acid sequence. Thus, a neoantigen differentiates a wildtype protein from its mutant-bearing tumor protein homolog, when such mutant is presented to T cells or B cells.

“Tumor specific antigen” or “tumor specific epitope” is used herein to designate an epitope or antigen that differentiates a mutated tumor protein from its unmutated wildtype homologue. Thus, a neoantigen is one type of tumor specific antigen.

As used herein “driver” mutations are those which arise very early in tumorigenesis and are causally associated with the early steps of cell dysregulation. Driver mutations are shared by all clonal offspring arising from the initial tumor cells and offer some additional fitness benefit to the clonal line within its microenvironment. In contrast passenger mutations are those somatic mutations which arise during the differentiation of the tumor and which offer no particular benefit of fitness to the cell. Passengers may serve as biomarkers on tumor cells and may enable some immune evasion. Passenger mutations may differ at different time points in its development and among different parts of a tumor or among metastases. “Driver and passenger” are terms largely interchangeable with “trunk and branch” mutations.

“Bespoke peptides” or “bespoke vaccine” as used herein refers to a peptide or neoantigen or a combination of peptides, or nucleic acid encoding peptides, that are tailored or personalized specifically for an individual patient, taking into account that patient's HLA alleles and mutations.

As used herein “TCGA” refers to The Cancer Genome Atlas (on the world wide web at cancer.gov/about-nci/organization/ccg/research/structural-genomics/tcga).

As used herein a “polyhydrophobic amino acid” refers to a short chain of natural amino acids which are hydrophobic. Examples include, but are not limited to, leucines, isoleucines or tryptophans where these are assembled in multimers of 5-15 repeats of any one such amino acid. As a non-limiting example, a poly leucine comprising 8 leucines would be an example of a polyhydrophobic amino acid.

A “lipid core peptide system”, as used herein, refers to subunit vaccine comprising a lipoamino acid (LAA) moiety which allows the stimulation of immune activity. A combination of T cell stimulating epitopes or T and B cell stimulating epitopes are linked to a LAA. Multiple different constructs can be created with of different spatial orientation or LAA lengths (e.g. C12 2-amino-D,L-dodecanoic acid or C16, 2-amino-D,L-hexadecanoic acid). When dissolved in a standard phosphate buffer LCP particles form and the particles facilitate uptake by antigen presenting cells. Different IAA chain lengths lead to different particle sizes.

As used herein, the term “cleavage site octamer” refers to the 8 amino acids located four each side of the bond at which a peptidase cleaves an amino acid sequence. Cleavage site octamer is abbreviated as CSO. “Cathepsin cleavage site octamer” is used herein where the peptidase is a cathepsin.

As used herein “compounding pharmacy” has the meaning defined in sections 503A and 503B of the Federal Food, Drug, and Cosmetic Act

As used herein, a “BAM” file is a compressed binary version of a Sequence Alignment File “SAM” file wherein all nucleotides are aligned to a reference genome. A “BAM slice” is a subset of the entire genome defined by genome coordinates. The HLA locus is located on Chromosome 6. In one particular instance a BAM slice is defined to contain just the HLA locus.

“Immunopathology” when used herein describes an abnormality of the immune system. An immunopathology may affect B-cells and their lineage causing qualitative or quantitative changes in the production of immunoglobulins. Immunopathologies may alternatively affect T-cells and result in abnormal T-cell responses. Immunopathologies may also affect the antigen presenting cells. Immunopathologies may be the result of neoplasias of the cells of the immune system. Immunopathology is also used to describe diseases mediated by the immune system such as autoimmune diseases. Representative autoimmune diseases include, but are not limited to rheumatoid arthritis, diabetes type I and type II, Ankylosing Spondylitis, Atopic allergy, Atopic Dermatitis, Autoimmune cardiomyopathy, Autoimmune enteropathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome, Autoimmune progesterone dermatitis, Autoimmune thrombocytopenic purpura, Autoimmune uveitis, Bullous Pemphigoid, Castleman's disease, Celiac disease, Cogan syndrome, Cold agglutinin disease, Crohns Disease, Dermatomyositis, Eosinophilic fasciitis, Gastrointestinal pemphigoid, Goodpasture's syndrome, Graves' disease, Guillain-Barré syndrome, Anti-ganglioside Hashimoto's encephalitis, Hashimoto's thyroiditis, Systemic Lupus erythematosus, Miller-Fisher syndrome, Mixed Connective Tissue Disease, Myasthenia gravis, Narcolepsy, Pemphigus vulgaris, Polymyositis, Primary biliary cirrhosis, Psoriasis, Psoriatic Arthritis, Relapsing polychondritis, Sjögren's syndrome, Temporal arteritis, Ulcerative Colitis, Vasculitis, and Wegener's granulomatosis.

“Antigen presenting cell” as used herein refers to cells which are capable of presentation of peptides to T cells bound to MHC molecules. This includes but is not limited to the so called “professional” antigen presenting cells comprising but not limited to dendritic cells, B cells, and macrophages, but also the so called non-professional antigen presenting cells which carry MHC molecules.

“Oncogene” as used herein is a gene which in certain circumstances can transform a cell into a tumor cell. A gene that, when activated by mutation, increases the selective growth advantage of the cell in which it resides[1]. Oncogenes may include both drivers, and also tumor suppressors which when inactivated by mutation increase selective advantage of a tumor cell. There are many documented oncogenes; these are catalogued in various databases such as the National Cancer Institute Genome Data Commons (on the world wide web at portal.gdc.cancer.gov/), Cosmic Catalogue of Somatic Mutations in Cancer (on the world wide web at cancer.sanger.ac.uk/cosmic). A few illustrative examples include, but are not limited to HER2 (ERBB2), EGFR, TP53, BRAF, KIT, PK3CA and PTEN.

“Adjacent oncogene” as used herein is used to refer to the oncogene positioned within 1 megabase of a bystander protein of interest.

As used herein “bystander protein” refers to a protein encoded in DNA adjacent to an oncogene, on either strand of DNA within about 1 megabase of the start or termination of the oncogene coding region. “co-amplified bystander protein” is used to describe a bystander protein which is overexpressed in conjunction with the over expression of the oncogene protein.

As used herein, gene acronyms are the HUGO (Human Genome Organization Gene Symbols) symbol, or variants thereof identified in Uniprot (on the world wide web at uniprot.org). EGFR as used herein refers to Epidermal growth factor receptor.

As used herein “GBM” is used as an abbreviation for glioblastoma multiforme

“Double minute” as used herein refers to small fragments of extrachromosomal DNA configured as circular DNA and lacking a centromere or telomere. Double minutes are also referred to herein as “DMs” and “dmins”

As used herein “ecDNA” refers to extrachromosomal DNA which occurs outside of chromosomes. ecDNA in cancer cells may comprise several Megabases of DNA

“SEC61G” and “SEC61gamma” or “SEC61γ” as used herein refers to the gene of that name and the protein encoded by the gene as exemplified by Uniprot sequence P60059

“VOPP”, which is also referred to as “ECOP” as used herein refers to the gene of that name and the protein “Vesicular, overexpressed in cancer, prosurvival protein 1” encoded by the gene and exemplified as Uniprot sequence Q96AW1

“LANCL2” and “LANC2” as used herein refers to the gene of that name and the protein LanC-like protein 2, encoded by the gene and exemplified by Uniprot sequence Q9NS86

“SEPT14” and “SEPTIN14” as used herein refer to the gene of that name and the protein Spetin-14 encoded by the gene and exemplified as Uniprot sequence Q6ZU15

As used herein “standardization” or “normalization” refers to a mathematical transformation of a data set to a normal or Gaussian distribution. Many data sets have distributions that are not normal and are variously skewed or kurtotic. Data sets may display various known distributions, such as log normal, exponential, gamma, Cauchy or Weibull. A SHASH (sinh-arcsinh) or Johnson Distribution transformation can be used to mathematically transform datasets to a to a normal or Gaussian distribution with a mean of zero and unit variance. This does not change the underlying data but merely converts the scale. Having done this, the transformed data can be submitted to various types of well-known statistical and probabilistic analyses.

As used herein “FPKM” or Fragments Per Kilobase per Million is a metric that described the number of sequencing reads of a sequence that contribute to determination of its sequence. Sequence-mapped alignments of exomic DNA or transcript RNA is transformed to a metric that is adjusted for the number of alignment reads, the length of the gene or transcript being mapped, and the total number of reads in the dataset. This transformation of the raw data takes into account a number of experimental variables. The FPKM data for both exons and mRNA transcripts is typically exhibits a log normal distribution.

As used herein “gnomAD’” refers to the genome aggregation database of known gene variant frequencies derived from in excess of 100,000 individuals. This database is housed at the Broad Institute (on the world wide web at broadinstitute.org/).

### DESCRIPTION OF THE INVENTION

There are a large number of well recognized oncogenes that play an important role in tumorigenesis as both drivers of tumor growth or as suppressors which may be silenced [1]. Focal amplification and gene rearrangements are characteristics of many cancer types [2, 3].

Sequence analysis of tumor biopsies in comparison to normal tissue identifies oncogenes that are upregulated, mutated and increased in copy number in tumors. There has been increasing interest in targeting epitopes in protein expressed from these with neoepitope vaccines. In some instances, and particularly where oncogene amplification is the result of multiplication of extrachromosomal DNA, genes encoded in close proximity of oncogenes are also upregulated and their protein products expressed. While not mutated, the proteins derived from these bystander genes may be prognostic indicators. In the present invention, we address the potential to target immune responses to the bystander gene products as a way to target a tumor cell. Where bystander genes are carried on extrachromosomal DNA they may occur in different combinations, and may vary in relative level of expression between different clonal lines of a tumor. However, in so far as they are expressed as companions to the oncogene product, they provide markers of the cells in which the oncogene is upregulated. In one embodiment of the present invention we identify T cell epitopes in particular such bystander proteins and identify peptides which may be used to stimulate a CD8+ cytotoxic T cell response, and peptides which may stimulate a CD4+ helper T cell response to the cells carrying the proteins. In a further embodiment we provide a method to modify the peptides to bind at a desired affinity to the specific HLA alleles which an affected subject may carry.

In some preferred embodiments of the present invention, we identify T cell epitopes, in particular in such bystander proteins, and identify peptides which may be used to stimulate a CD8+ cytotoxic T cell response, and peptides which may stimulate a CD4+ helper T cell response to the cells carrying the proteins. In some further preferred embodiments, we provide methods to modify the peptides to bind to specific HLA alleles which an affected subject may carry. In still other preferred embodiments, we provide target epitopes in bystander proteins located in chromosome 7 adjacent to EGFR. Also provided is a method of simultaneous targeting of peptides in the bystander proteins and the oncogene, where the latter is mutated. In a particular embodiment a method of targeting a combination of chromosome7 bystander protein and mutated EGFR is provided. However, this example is not considered limiting as bystander proteins may be associated with oncogene upregulation in cancers in which EGFR is not a dominant oncogene.

### Double Minutes

Extrachromosomal DNA (ecDNA) configured as circular “double minutes” (DMs or dmins) are common in cancer although their precise genesis is poorly understood [3, 5, 6]. DMs are considered an important mode of extrachromosomal genomic amplification with a key role in tumorigenesis. ecDNA is documented in about half of glioblastomas, but also in many other cancer types, including but not limited to, neuroblastoma, melanoma, colon, breast, ovarian, lung, renal, hemopoietic, hepatic, prostate, pancreatic, and colon cancers, and medulloblastoma [3, 7-13]. Depending on the genes encoded, the autonomous replication of ecDNA comprising oncogenes, which may be followed by chromosomal re-integration, a process which may be repeated many times. This results in amplification of the oncogenes, and other adjacent encoded genes, and may enhance the fitness of tumor cells, thereby advancing tumorigenesis. Glioblastomas commonly comprise tumor cells with DMs. When these express EGFR they are reported to be more invasive. DMs expressing MYC, PDGFRα, HER2 (ERBB2), CDK4, and MDM2 have also been reported in GBM [10]. In neuroblastoma MYCN is reported on DMs [14]. In colon cancer dihydrofolate reductase (DHFR) gene amplification on ecDNA is common. In ovarian cancer, or cells derived therefrom, MYCN is reported to occur on ecDNA and in breast cancer HER2 may be amplified on ecDNA [12, 13].

Some DMs comprise up to several megabases of DNA. Hence they large enough to carry one or more complete genes. The combination of these genes and the functionality of their expression, depends on the location of DNA breakpoints in the formation of DMs. Thus, every tumor may have a different combination of adjacent bystander genes expressed from DMs and different cells and clonal lines within the tumor may express different combinations of proteins therefrom. DMs tend to result in high levels of transcription and expression. In some instances, the coamplified gene products may be passive bystanders, whereas in other cases they may play a role in enhancing tumorigenesis.

### EGFR

The upregulation of EGFR is documented in many cancers, including but not limited to cancers of bronchus and lung, skin, uterus, ovary, brain, stomach, hematopoietic and reticuloendothelial systems, colon, breast, bladder, liver, adrenal, prostate and others. EGFR upregulation is common feature of the classical form of glioblastoma [15-18]. In glioblastoma the upregulation is often accompanied by upregulation of functional splice variants EGFRvIII (deletion of exons 2-7), and vii (deletion of exons 14-15) [15]. Point mutations are also frequently observed in EGFR in glioblastoma in the extracellular region. All of these aberrant forms are constitutively active. A number of mutations are characteristic of GBM, whereas in other cancers EGFR exhibits other common mutations. For example, mutation L858R is observed in some in non-small cell lung cancers. EGFRvIII is typically expressed in tumor tissue in GBM but not normal tissue and hence is the target of therapy. As EGFR is often encoded on ecDNA and double minutes copies of EGFR may accumulate in tumor cells, and different clonal lines take on different characteristics with respect to their EGFR copy number and proportion of normal and splice variant forms. The relative balance of each clonal line and EGFR content then continues to fluctuate in the face of surgical, radiation, drug and immunotherapeutic interventions [18, 19].

### Other Chromosome 7 Encoded Proteins

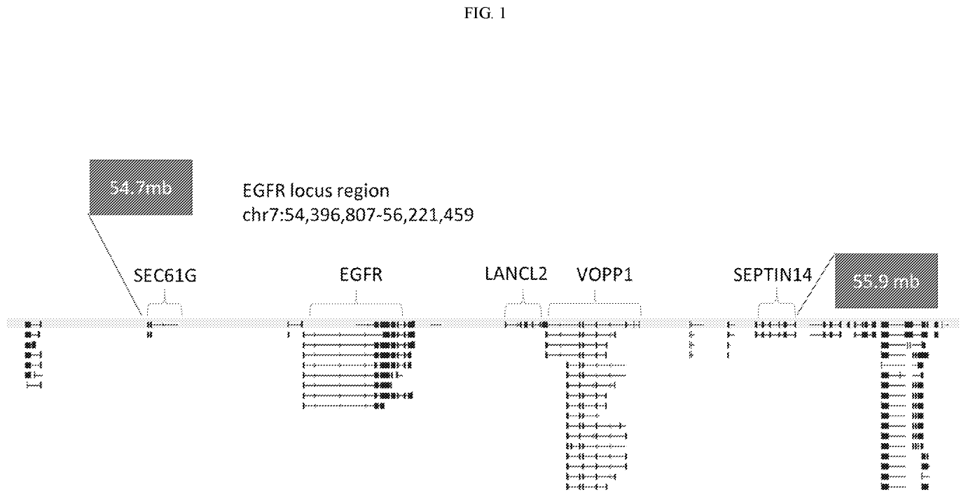

In one embodiment of the present invention, we identify genes encoded on chromosome 7 adjacent to EGFR and the T cell epitopes in these proteins. In some tumors these genes are upregulated and transcribed along with EGFR, either on extrachromosomal DNA, directly from chromosomal DNA, or following reintegration of ecDNA into chromosomal DNA. The bystander genes encoded on chromosome 7 close to EGFR include VOPP, SEC61, LANCL2 and SEPT14. FIG. 1 shows the relative positions of these genes on chromosome 7. Breaks in this region of chromosome 7 may produce chromosome fragments containing a combination of some, or all, of SEC61G, EGFR, LANCL2, SEPT14 and VOPP1 that may be incorporated into ‘double minute’ circular chromosomal fragments in the cytoplasm of tumor cells. The breaks occur in slightly different locations in different tumors, but those that have been mapped are between the 53.5 and 56 megabase coordinates of chromosome 7. The resultant DNA fragments may encode all 4 proteins or just some of them. Lu et al showed that in examination of 43 GBM tumors 77% expressed SEC61G at significantly higher levels than normal brain tissues, and the other genes in LANCL2, and VOPP, showed significant overexpression [20]. Expression of SEC61G is also seen as a poor prognostic marker for GBM cases [21].

We identify T cell epitopes in SEC61G, LANCL2, SEPT14 and VOPP1 and provide synthetic peptides, which when applied to a subject in which these proteins are upregulated, provides a means of targeting an immune response to tumor cells bearing the proteins. In preferred embodiments the immune response is a CD8+ T cell cytotoxic response and in further preferred embodiments a CD8+ response is accompanied by a CD4+ driven T helper response.

### Copy Number Variation Analysis

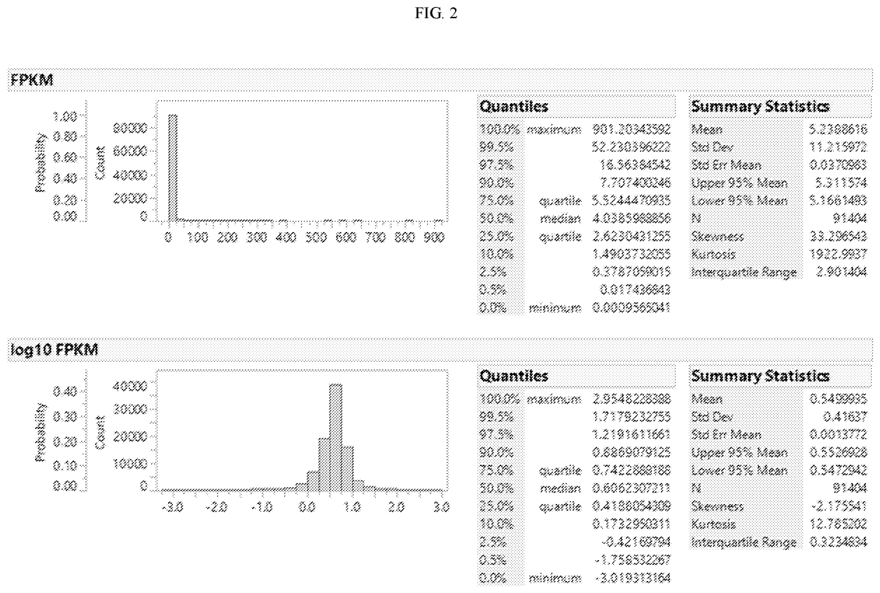

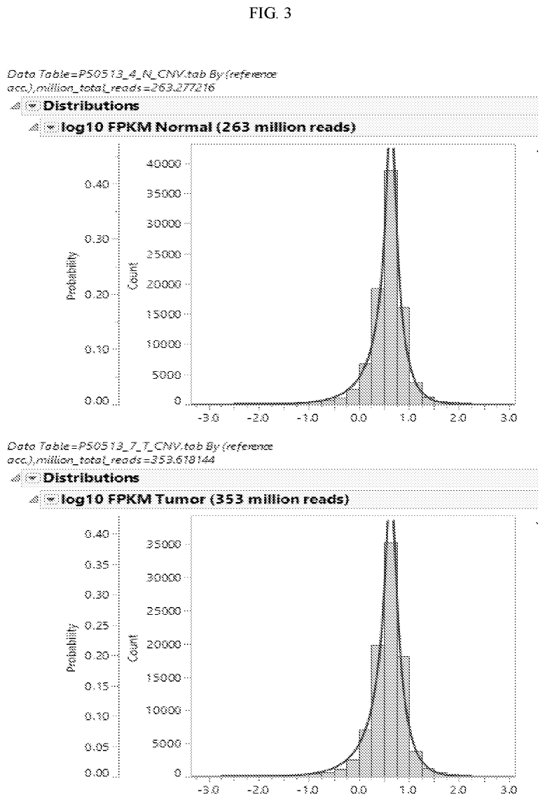

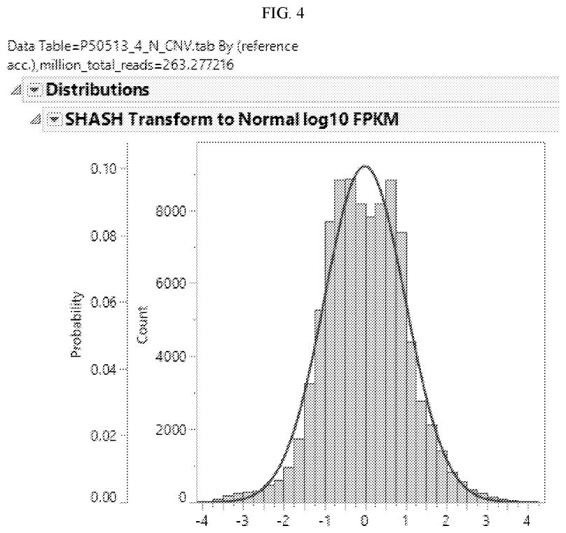

DNA and RNA sequencing is conducted from tumor biopsies and from normal tissue of the subject, typically from blood cells. Sequence-mapped alignments of exomic DNA or transcript RNA is transformed to a metric that is adjusted for the number of alignment reads, the length of the gene or transcript being mapped, and the total number of reads in the dataset. This is termed “FPKM” or Fragments Per Kilobase per Million reads. This transformation of the raw data takes into account a number of experimental variables. The FPKM data for both exons and mRNA transcripts is typically exhibits a log normal distribution. This is illustrated in FIGS. 2-4.

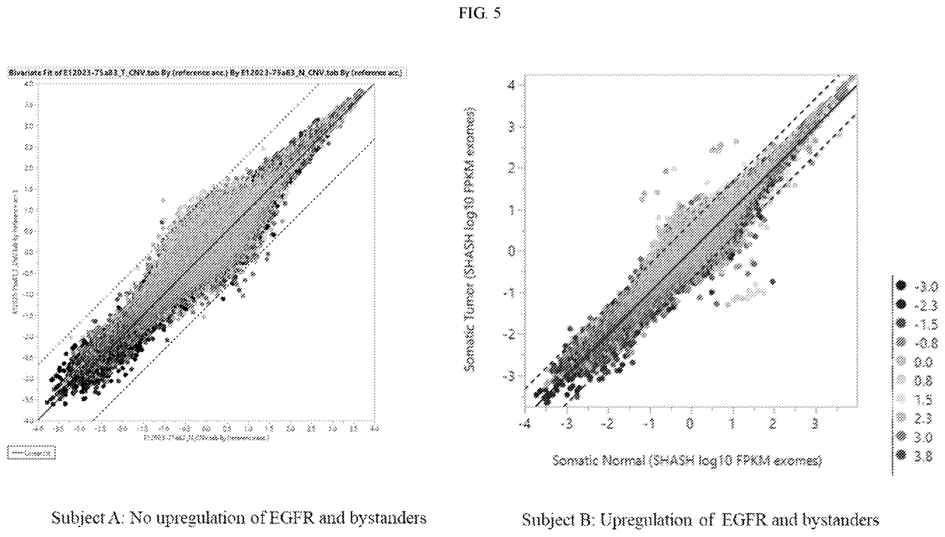

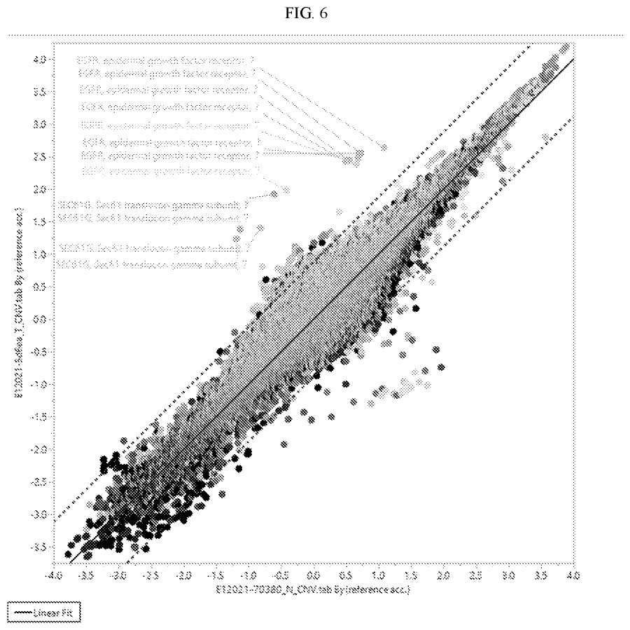

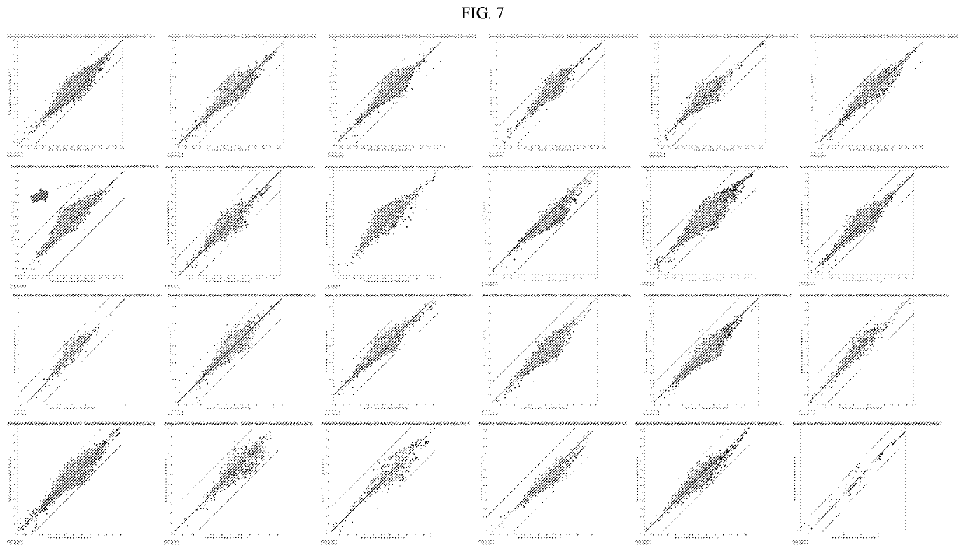

FIG. 5 shows an example of copy number comparison between tumor and normal for a GBM patient in which upregulated EGFR and coamplified SEC61G proteins are clearly observed compared to a comparison of tumor normal from a different GBM subject in which EGFR is not upregulated. Each datapoint represents the paired comparison of the tumor and normal copy number with the value being that of the normalized FPKM of one unique transcript (ENST) defined in GCRh38. In addition, in this graphic each point is colored based on the mRNA transcript enumeration in the tumor biopsy using the same normalization methodology (see scale on right side of EGFR upregulated subject). The regression line is has a constrained slope=1 and intercept=0. Thus, any point that has the same standardized value in the tumor and normal will fall on the line. The RMSE (root mean squared error) for the regression is calculated to be 0.25. The dashed line is the confidence limit around the regression line with alpha=0.01 and thus 99% of values will fall within the boundaries. The outlier points above the line are read alignments with different ENST for EGFR and SEC61G that that form double minutes and are upregulated in this patient. These are identified in FIG. 6. Points below the line are alignments that have been deleted and thus being much lower in the exomes despite being expressed at an above average level of 0.8 (mRNA coloration). The copy number differential is computed as the residuals from the regression line. The Studentized residuals, the actual residual divided by the RMSE provides a probabilistic estimate of the copy number differential. The studentized values for SEC61 and EGFR have values in the range of 8-9 or are 8-9 standard deviations outside the line. As shown this analysis is for the entire genome. Such examples can be restricted to a chromosome or a chromosomal region if desired. An example of an individual chromosomal comparison is shown in FIG. 7, where only chromosome 7 shows a significant number of upregulated.

### Selection of Epitopes Based on Patient HLA Alleles

One embodiment of this invention is to provide synthetic peptides which will elicit a CD8+ or a CD4+ immune response to an epitope in a tumor comprising an upregulated gene. Computational methods for identifying HLA alleles of a subject from the whole exome sequence are known to those skilled in the art [22, 23] (See, e.g., PCT US2020/037206, which is incorporated by reference herein in its entirety). Peptide epitopes are presented for binding to T cell receptors when bound into MHC molecular grooves. Binding affinity of any given peptide varies between HLA allele. The present inventors have developed algorithms based on principal component analysis of multiple amino acid physical and chemical properties which provide accurate predictions of MHC I and MHCII peptide binding (See, e.g., PCT US2011/029192, PCT US2012/055038, US2014/014523, PCT US2015/039969, PCT US2017/021781, US Publ. No. 20130330335, US Publ. No. 20160132631, US Publ. No. 20170039314, US Publ. No 20170161430, US Publ. No. 20190070255, PCT US2020/037206, U.S. Pat. No. 10,706,955 and 10,755,801, each incorporated by reference herein in its entirety). In particular embodiments of the present invention therefore, the amino acid sequences of the four proteins encoded adjacent to EGFR, which may be co-expressed as co-amplified bystanders, were analyzed to identify peptides which, when delivered as a synthetic peptide immunogen, could provide MHC binding and optimum stimulation of CD8 or CD4 T cells across a broad range of alleles. These are described in the following Examples. For individual subjects bearing some HLA alleles, synthetic peptides were designed to optimize binding to particular HLA alleles over that naturally occurring in the native protein. Examples of such “personalized” synthetic peptides are also described.

While the examples that follow apply to epitopes carried by those proteins encoded and upregulated as co-amplified companions to EGFR, either intra or extra-chromosomally, the examples also provide a road-map for how to approach design of a synthetic peptide vaccine to stimulate T cells directed to epitopes on other proteins, which may be upregulated and coamplified as bystanders or companions to other oncogenes amplified in cancers. In some particular embodiments such coamplified proteins are encoded on DMs, in yet others they are encoded in other forms of ecDNA or intrachromosomally. Hence the examples that follow are not considered limiting.

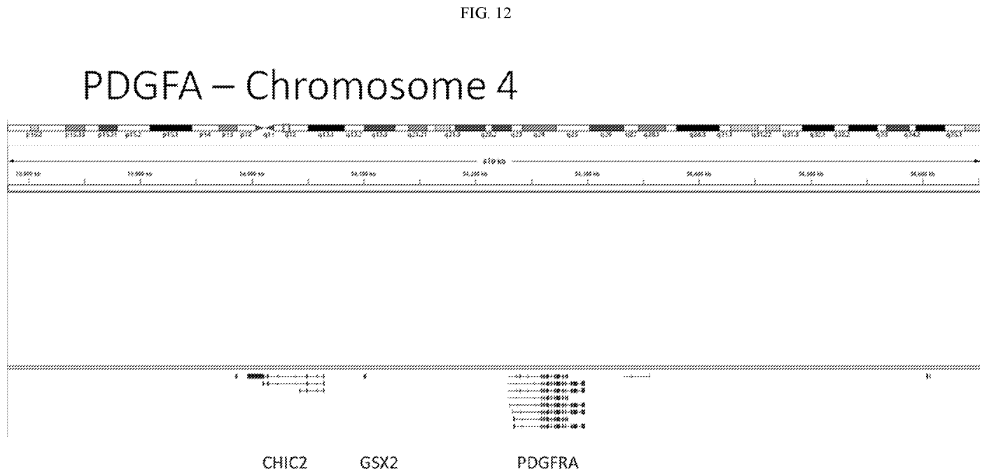

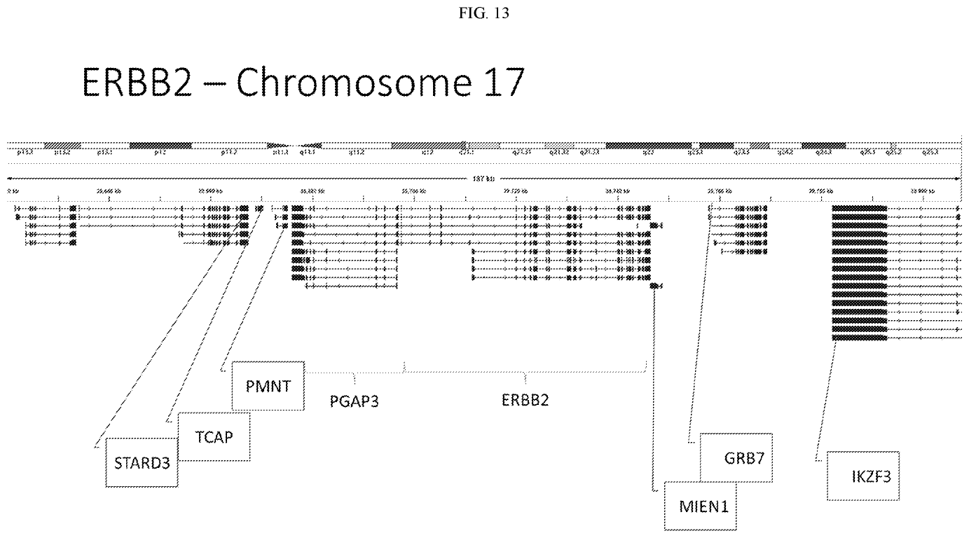

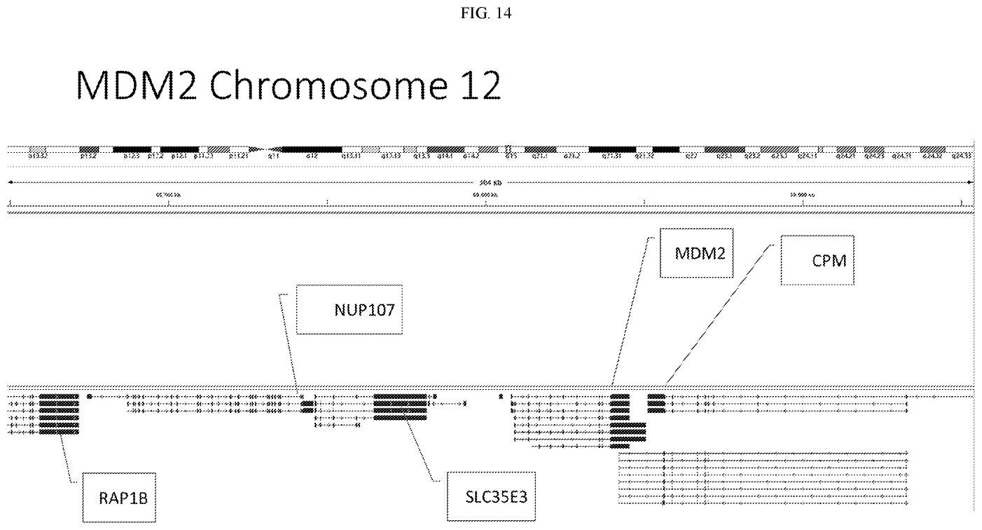

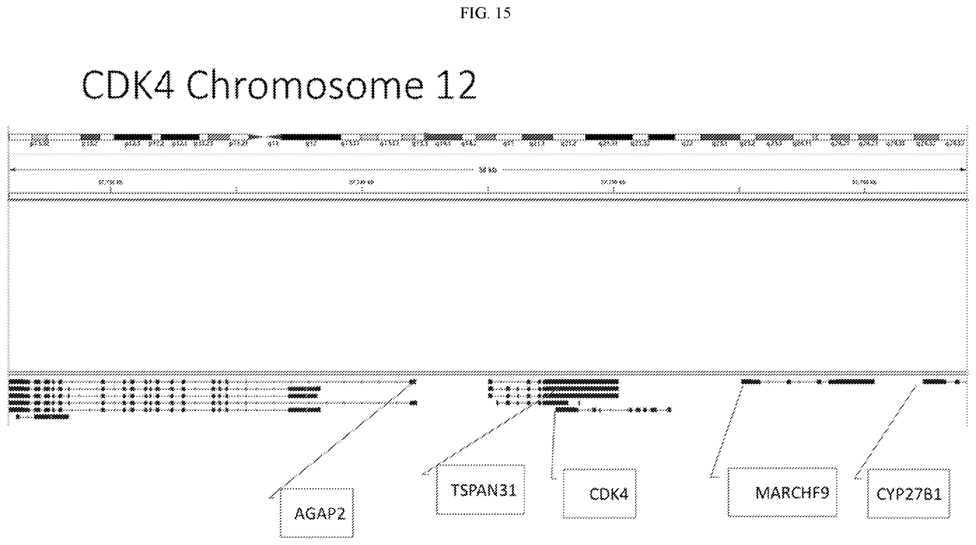

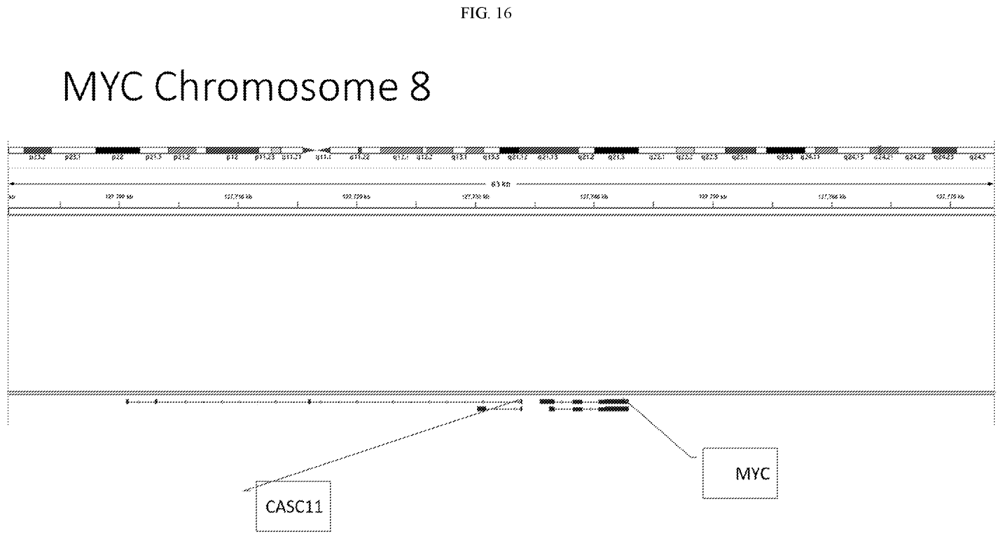

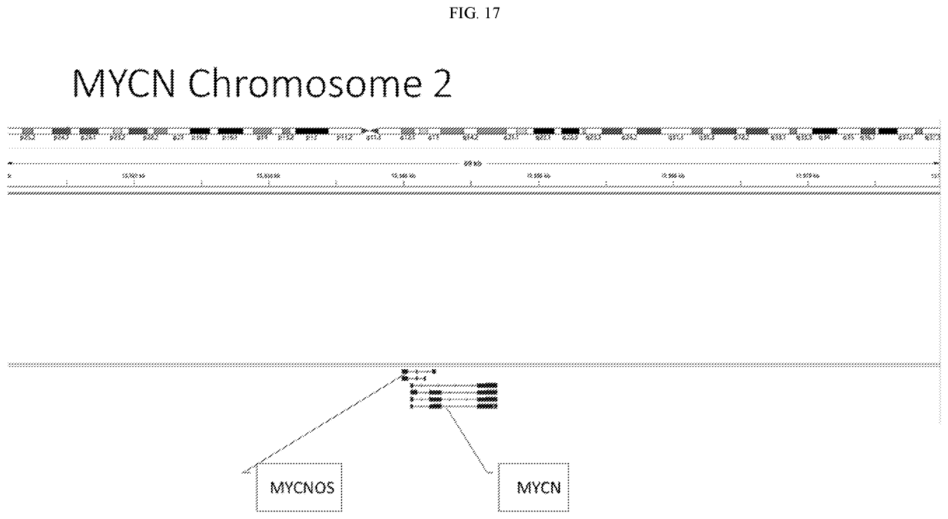

FIGS. 12-17 provide examples of other bystander proteins which may be targeted as co-amplified bystanders in chromosome 4 adjacent to PDGFA, chromosome 17 adjacent to ERBB2 (HER2), chromosome 12 adjacent to MDM2, chromosome 12 adjacent to CDK4, chromosome 8 adjacent to MYC, and chromosome 2 adjacent to MYCN.

The objective of vaccination with coamplified proteins, co-expressed and co-upregulated with oncogenes, such as EGFR, is to direct a cellular immune response to destroy tumor cells carrying such proteins. It follows that another embodiment is thus to vaccinate with synthetic peptides, or the nucleotide sequences that encode them, from a multiplicity of such proteins that are co-expressed or a multiplicity of epitopes derived from the proteins. Further in another embodiment the invention provides for vaccination of a subject simultaneously with peptide epitopes, or their encoding nucleic acids, derived from both the oncogene protein and the co-amplified proteins.

In some embodiments of the present invention, when used as a vaccine the peptides selected from the proteins of interest may be delivered parenterally. In some particular embodiments, delivery is intradermally, by injection or microneedle array, or subcutaneously. In yet other embodiments the selected peptides are delivered non-parenterally to a mucosal surface and in some preferred embodiments are delivered orally. However, the selected peptides may be administered to the subject by any route deemed appropriate by the clinician. The peptides may be applied in conjunction with an adjuvant or local inflammatory agent. Peptides may be suspended in a pharmaceutically acceptable carrier. In some embodiments, peptides may be formulated to enhance uptake by antigen presenting cells, especially dendritic cells, This may be by inclusion of an adjuvant in the formulation administered; such an adjuvant may be drawn from the group comprising, but not limited to, polyI.CLC, montanide, GM-CSF, imiquimod or any other pharmaceutically acceptable adjuvant. In some embodiments, peptide application to the subject may be followed by a checkpoint inhibitor or other immunomodulatory intervention. The peptides may also be used in vitro to prime autologous dendritic cells or T cells that are then administered to the patient.

The immune response to bystander protein epitopes such as those descried here may be monitored by assays of T cell responses including but not limited to ELISPOT assays and monitoring of T cell repertoires. Hence in a further embodiment, the peptides described as epitopes in bystander gene products are also constituents of a diagnostic kit for monitoring the progress of the immune response to a tumor.

### Sequence Analysis

Certain embodiments described above require analysis of the protein sequences contained within a biopsy from a subject.

In some preferred embodiments, mutated proteins in biopsy samples are identified by sequencing the genome, proteome or transcriptome of cells from the biopsy. The present invention is not limited to any particular method of obtaining sequences of mutated in a biopsy. A variety of sequencing methods are readily available to those of ordinary skill in the art.

In some preferred embodiments, the present invention utilizes nucleic acid sequencing techniques. The nucleic acid sequences are preferably converted in silico to protein sequences from the identification of mutated amino acids and peptides comprising the mutated amino acids.

In some embodiments, the sequencing is Second Generation (a.k.a. Next Generation or Next-Gen), Third Generation (a.k.a. Next-Next-Gen), or Fourth Generation (a.k.a. N3-Gen) sequencing technology including, but not limited to, pyrosequencing, sequencing-by-ligation, single molecule sequencing, sequence-by-synthesis (SBS), semiconductor sequencing, massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety. Those of ordinary skill in the art will recognize that because RNA is less stable in the cell and more prone to nuclease attack experimentally RNA is usually reverse transcribed to DNA before sequencing.

DNA sequencing techniques include fluorescence-based sequencing methodologies (See, e.g., Birren et al., Genome Analysis: Analyzing DNA, 1, Cold Spring Harbor, N.Y.; herein incorporated by reference in its entirety). In some embodiments, the sequencing is automated sequencing. In some embodiments, the sequencing is parallel sequencing of partitioned amplicons (PCT Publication No: WO2006084132 to Kevin McKernan et al., herein incorporated by reference in its entirety). In some embodiments, the sequencing is DNA sequencing by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; U.S. Pat. Nos. 6,432,360, 6,485,944, 6,511,803; herein incorporated by reference in their entireties), the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. Nos. 6,787,308; 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), and the Adessi PCR colony technology (Adessi et al. (2000). Nucleic Acid Res. 28, E87; WO 00018957; herein incorporated by reference in its entirety).

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), Life Technologies/Ion Torrent, the Solexa platform commercialized by Illumina, GnuBio, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. Nos. 6,210,891; 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 106 sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. Nos. 6,833,246; 7,115,400; 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 250 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. Nos. 5,912,148; 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, sequencing is nanopore sequencing (see, e.g., Astier et al., J. Am. Chem. Soc. 2006 Feb. 8; 128(5):1705-10, herein incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

In certain embodiments, sequencing is HeliScope by Helicos BioSciences (Voelkerding et al., Clinical Chem., 55: 641-658, 2009; MacLean et al., Nature Rev. Microbiol., 7: 287-296; U.S. Pat. Nos. 7,169,560; 7,282,337; 7,482,120; 7,501,245; 6,818,395; 6,911,345; 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is −99.6% for 50 base reads, with −100 Mb to 100 Gb generated per run. The read-length is 100-300 base pairs. The accuracy for homopolymer repeats of 5 repeats in length is −98%. The benefits of ion semiconductor sequencing are rapid sequencing speed and low upfront and operating costs.

In some embodiments, sequencing is the technique developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 20090035777, entitled “High Throughput Nucleic Acid Sequencing by Expansion,” filed Jun. 19, 2008, which is incorporated herein in its entirety.

Other emerging single molecule sequencing methods include real-time sequencing by synthesis using a VisiGen platform (Voelkerding et al., Clinical Chem., 55: 641-58, 2009; U.S. Pat. No. 7,329,492; U.S. patent application Ser. No. 11/671,956; U.S. patent application Ser. No. 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

In other preferred embodiments, the present invention utilizes protein sequencing techniques. In some embodiments, proteins may be sequenced by Edman degradation. See, e.g., Edman and Begg (1967). “A protein sequenator”. Eur. J. Biochem. 1 (1): 80-91; Alterman and Hunziker (2011) Amino Acid Analysis: Methods and Protocols. Humana Press. ISBN 978-1-61779-444-5. In other embodiments, mass spectrometry techniques are utilized to sequence proteins. See, e.g., Shevchenko et al., (2006) “In-gel digestion for mass spectrometric characterization of proteins and proteomes”. Nature Protocols. 1 (6): 2856-60; Gundry et al., (2009) “Preparation of proteins and peptides for mass spectrometry analysis in a bottom-up proteomics workflow” Current Protocols in Molecular Biology. Chapter 10: Unit 10.25.

### EXAMPLES

### Example 1: SEC61Gamma

SEC61G (gamma) is 68 amino acid protein comprising a transmembrane domain that is a subunit of the SEC61 pore-forming translocon complex that mediates transport of signal peptide-containing precursor polypeptides into the endoplasmic reticulum lumen (uniprot.com)[24]. Only a single isoform of SEC61G is recognized. SEC61G is encoded on chromosome 7 0.7 megabases upstream (5′) on same (positive) strand of DNA as EGFR.

Lu et al noted that SEC61G is upregulated in a large proportion of glioblastomas [20] but not in lower grade gliomas. They noted upregulated EGFR was almost always accompanied by upregulation of SEC61G. In vitro siRNA mediated knockdown of SEC61G led to growth suppression, increased apoptosis and cell death. It appears that SEC61G may serve a role in facilitating cell survival in GBM as part of a stress adaptive response to the hypoxic tumor microenvironment. Knock down of SEC61G can therefore lead to increased tumor cell apoptosis. SEC61G also appears to play a role in EGFR trafficking and activation of the PIK3-AKT pathway [25]. High expression of SEC16G is an indicator of poor prognosis in GBM.[21]. In another report a SEC61G-EGFR fusion was reported [26]. These observations point to SEC61G as a potential target for pharmaceutical intervention, and also indicates that immune targeting of SEC61G may facilitate knock out of EGFR over expressing cells.

Examination of GBM patients with upregulated EGFR, showed upregulation of Sec61G. Examples are shown in FIGS. 5-7.

That peptides from SEC61G may be presented on MHC was demonstrated by Neidert et al, [27] who, by using mass spectroscopy, detected peptide IHIPINNII bound to MEC I B38. Analysis by the present inventors indicated that this peptide was predicted to bind to MHC I B38 with extremely high affinity, in the top 1.5% or all peptides in the protein. It is fairly typical that mass spectroscopy will detect primarily the highest affinity MHC binders. However, such peptides may not be the optimum to provide T cell stimulation. This published example of a high binding peptide for one relatively less common MHC I allele therefore teaches away from identification of epitope peptides with optimal binding for a broad array of MHC I and MHC II alleles to stimulate a T cell response.

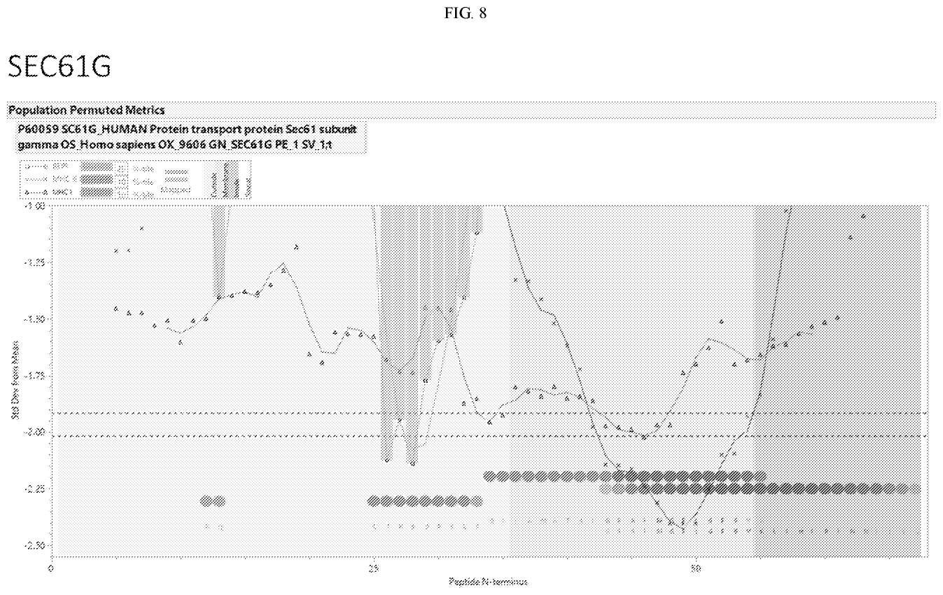

FIG. 8 provides an overview map of the MHC I and MHC II binding within SEC61G, showing the highest binding peptides are found in the transmembrane domain. Analysis of the predicted binding of each sequential 9mer and 15 mer peptide in SEC61G was conducted using methods previously described (see, e.g., U.S. Ser. No. 10/706,955, incorporated herein by reference in its entirety). Tables 1 and 2 show the peptides in SEC61G with highest predicted binding affinity for MHC I and MHC II alleles which comprise desirable peptides for inclusion in a vaccine composition. Analysis was conducted for 31 MHC I A, 31 MHC I B alleles and 8 MHC I C alleles, as well as for 24 DRB alleles. In the interest of space only a subset of the results are shown in Tables 1 and 2. The peptides identified may be synthesized and applied to the subject to be vaccinated as individual 9 mer or 15 mers according to the specific alleles of an individual subject or may be administered as a longer peptide comprising one or more of the peptide sequences shown in Tables 1 and 2. In some particularly desired embodiments, the peptides have a higher probability of being excised by cathepsin L or S, as shown in Tables 1 and 2, and thus more readily processed for presentation by antigen presenting cells.

In the case of a few HLA alleles, peptides with a desirable binding affinity are not found among the sequences shown in Tables 1 and 2. In such instances a customized synthetic peptide may be created to optimize MHC I binding and T cell stimulation by retaining the T cell exposed motif engaged by the T cell receptor unchanged but changing the amino acids that lie in the MHC groove exposed motifs or pocket positions so as to enhance binding. Table 3 shows examples of synthetic peptides designed to elicit a MHC I CD8+ response to SEC61G for alleles A2601 and A3201. These alleles were selected as representative examples and thus are not considered limiting.

### Example 2: VOPP

VOPP is the acronym of the Vesicular, overexpressed in cancer, pro-survival protein 1. Alternative names for the same protein are ECOP (EGFR-coamplified and overexpressed protein) and GASP (Glioblastoma-amplified secreted protein). This 172 amino acid protein (canonical isoform) is expressed on chromosome 7 just downstream of EGFR and from the opposite DNA strand. There are multiple shorter isoforms, which share certain epitopes with the longer canonical and validated isoforms. VOPP was first described by Park et al [28] as a protein which regulated NF-kB transcriptional activity and resistance to apoptosis. The effect on the NF-kB pathway has been questioned by others, although there is agreement that there is a pro-survival effect of VOPP expression on cells [29]. When VOPP was down-regulated cellular susceptibility to apoptosis increased, suggesting that in tumors it may also contribute to resistance to apoptosis. VOPP is overexpressed in at least 33% of GBM [30] and its expression has been shown in squamous carcinoma cells [31] where it also confers protection against apoptosis. VOPP1 is also highly expressed in several other common human cancers, including breast carcinoma, pancreatic carcinoma, and lymphoma [29].

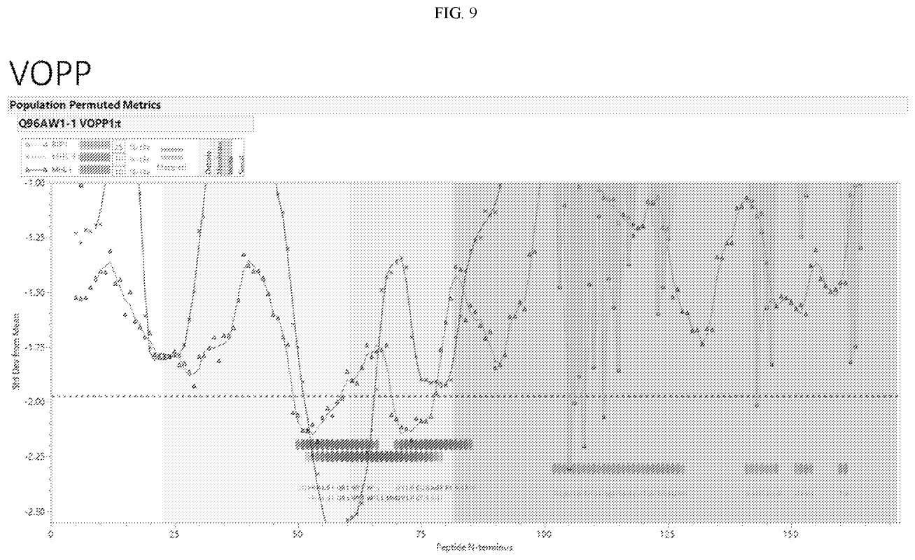

FIG. 9 provides an overview map of the MHC I and MHC II binding within VOPP1, showing the highest binding peptides are found in the transmembrane domain and at the N terminal of the mature protein.

Tables 4 and 5 show the peptides in VOPP with highest predicted binding affinity for MHC I and MHC II alleles which comprise desirable peptides for inclusion as synthetic peptides in a vaccine composition. Analysis was conducted for 31 MHC I A, 31 MHC I B alleles and 8 MHC I C alleles, as well as for 24 DRB alleles. In the interest of space only a subset of the results are shown in Tables 4 and 5. Peptides may be selected as individual 9 mer or 15 mers according to the specific alleles of an individual subject or may be administered as a longer synthetic peptide comprising one or more extensions of the sequential peptide sequences shown in Tables 3 and 4. In some particularly desired embodiments, the peptides have a high probability of being excised by cathepsin L or S and thus more readily processed for presentation by antigen presenting cells. VOPP occurs as multiple isoforms (Uniprot Q96AW1 Q96AW1-2 Q96AW1-3 Q96AW1-4) however the sequences identified in Tables 4 and 5 as desirable synthetic vaccine components are in the conserved regions of the protein.

In the case of a few HLA alleles, peptides with a desirable binding affinity are not found among the naturally occurring sequences shown in Tables 4 and 5. In such instances a customized peptide may be created to optimize MHC I binding and T cell stimulation for a particular subject by retaining the T cell exposed motif constant, but changing amino acids that lie in the MHC groove exposed motifs or pocket positions. Table 6 shows examples of synthetic peptides designed to elicit an MHC I CD8+ response to VOPP for alleles A3001 and A1101. These alleles were selected as representative examples and thus are not considered limiting.

### Example 3: LANC2

LANC2, Lanthionine Synthetase Components (LanC)-like protein 2 (also referred to as LANCL2) is expressed from chromosome 7 in close proximity to, and downstream from, EGFR on the same DNA strand. It is a 450 amino acid protein with a single validated isoform. LANC2 appears to have a function in the activation of abscisic acid binding on the cell membrane and the ABA signaling pathway in granulocytes. It has been recognized as a coamplified bystander which is overexpressed with EGFR in about 20% of glioblastomas and has been shown to change sensitivity of cells to the anticancer drug adriamycin [32].

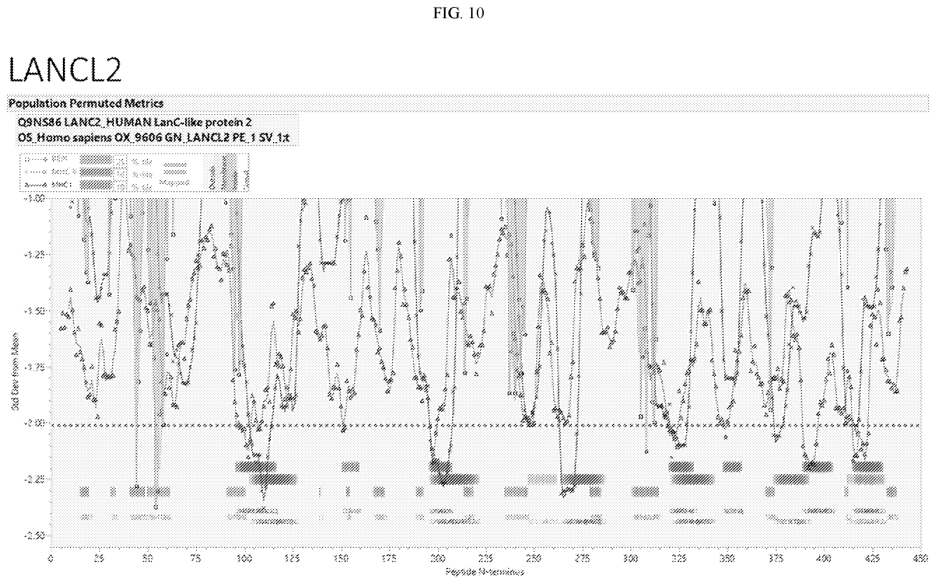

FIG. 10 provides an overview map of the MEC I and MHC II binding within LANC2, showing the highest binding peptides are found in the transmembrane domain and at the N terminal of the mature protein.

Tables 7 and 8 show the peptides in LANC2 with highest predicted binding affinity for MEC I and MHC II alleles which comprise desirable peptides for inclusion in a synthetic vaccine composition. Analysis was conducted for 31 MHC I A, 31 MHC I B alleles and 8 MHC I C alleles, as well as for 24 DRB alleles. In the interest of space only a subset of the results are shown in Tables 7 and 8. Peptides may be selected as individual 9 mer or 15 mers according to the specific alleles of an individual subject or may be administered as a longer peptide comprising one or more extensions of the sequential peptide sequences shown in Tables 7 and 8. In some particularly desired embodiments, the peptides have a higher probability of being excised by cathepsin L or S and thus natural presentation by antigen presenting cells. In the case of a few alleles, peptides with a desirable binding affinity are not found among the sequences shown in Tables 7 and 8. In such instances a customized peptide may be created to optimize MHC I binding and T cell stimulation for a particular subject by retaining the T cell exposed motif constant but changing the amino acids that lie in the MHC groove exposed motifs or pocket positions. Table 9 shows examples of synthetic peptides designed to elicit an MHC I CD8+ response to LANC2 for alleles A0801, A0217, A 3101 and A3301. These alleles were selected as representative examples and thus are not considered limiting.

### Example 4. Septin 14

Septin14 (SEPTIN14 or SEPT 14) is a fourth protein located close to EGFR on chromosome 7, which has been reported to be upregulated in brain [33] and as a fusion expressed with EGFR in lung cancer [34]. It is recognized in a single isoform of 432 amino acids encoded on chromosome 7.

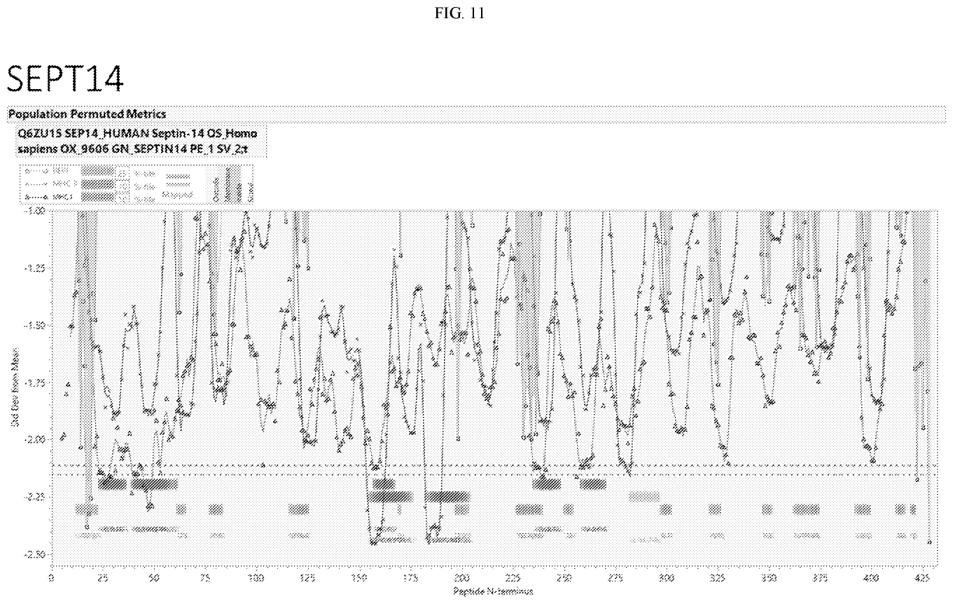

FIG. 11 provides an overview map of the MHC I and MHC II binding within SEPTIN14, showing the highest binding peptides are found in the transmembrane domain and at the N terminal of the mature protein. Tables 10 and 11 show the peptides in SEPTIN14 with highest predicted binding affinity for MHC I and MHC II alleles which comprise desirable peptides for inclusion in a vaccine composition. These may be selected as individual 9 mer or 15 mers according to the specific alleles of an individual patient or may be administered as a longer peptide comprising one or more extensions of the sequential peptide sequences shown in Tables 10 and 11 In some particularly desired embodiments the peptides selected from SEPTIN14 have a higher probability of being excised by cathepsin L or S and natural presentation by antigen presenting cells.

### Example 5: Epitopes in Combination with EGFR

As the 4 proteins noted in examples 1˜4 are co-expressed with EGFR, the peptides identified for use as components of a synthetic vaccine may be combined with synthetic peptides targeting EGFR itself. In preferred embodiments such peptides from EGFR comprise tumor specific T cell epitopes. Such epitopes may be tumor specific by inclusion of a mutation unique to the particular subject or the unique epitopes which arise because of the presence of a tumor associated variant of EGFR such as EGFR vIII or vII. Mutations commonly reported in EGFR include A289V, A289D, A289T and G598V or G598D in glioblastomas and L585R in lunch cancer. Table 12 shows the T cell exposed motifs which are tumor specific and associated with these mutations and those arising from the common vIII variant. However individual subjects may also carry “personal” mutations in EGFR which are not widely shared as the above examples are. In these cases a neoepitope vaccine may be designed to encompass the T cell exposed motifs of those particular mutations. In each of these cases the flanking amino acids comprising the groove exposed motifs may or may not provide a desired level of binding to the MHC of the affected subject. If a naturally occurring peptide comprising a tumor specific mutation is present it may be used in its natural form. Where such binding is not anticipated, a customized peptide may be designed to achieve a synthetic peptide with binding customized to the particular subject. An illustrative example provided in Table 13 for a subject with an EGFR vIII variant, to encompass the unique T cell exposed motif but binding for a representative group of MHC I alleles. This example for representative alleles is not considered limiting, as the same approach can be applied to provide a synthetic vaccine peptide targeting other individual tumor specific mutations in EGFR.

TABLE 1
Predicted binding of peptides of SEC61G to representative MHC I A and MHC IB alleles

| | | | TCEM | | | | | | | | | | | | |
|---|---|---|---|---|---|---|---|---|---|---|---|---|---|---|---|
| | SEQ | | SEQ | | | Predicted binding of representative MHC I A alleles ### | | | | | | | | | |
| | ID | | ID | Cat | Cat | A_ | A_ | A_ | A_ | A_ | A_ | A_ | A_ | A_ | A_ |
| 9-mer | NO.: | TCEMI # | NO.: | S## | L## | 0101 | 0201 | 0202 | 0203 | 0206 | 0211 | 0212 | 0301 | 0801 | 1101 |
| IMGFIGFFV | 1 | ~~~FIGFF~ | 13 | No | Yes | −2.16 | −2.48 | −1.61 | −1.21 | −1.24 | −1.04 | −1.48 | −0.43 | −0.36 | −0.05 |
| MGFIGFFVK | 2 | ~~~IGFFV~ | 14 | No | No | −0.78 | 0.40 | 0.65 | 0.92 | 0.30 | 0.16 | 0.61 | −2.84 | 0.56 | −2.66 |
| GFIGFFVKL | 3 | ~~~GFFVK~ | 15 | No | No | 1.09 | −1.25 | −1.62 | −1.48 | −1.27 | −1.36 | −2.01 | −0.14 | −0.42 | −0.74 |
| FIGFFVKLI | 4 | ~~~FFVKL~ | 16 | No | No | −1.21 | −1.93 | −2.73 | −1.86 | −0.94 | −0.89 | −1.11 | 0.09 | −0.66 | 0.97 |
| IGFFVKLIH | 5 | ~~~FVKLI~ | 17 | No | Yes | −1.24 | 0.39 | 0.71 | 0.75 | 0.31 | 0.67 | 0.32 | −1.13 | 0.59 | −1.32 |
| GFFVKLIHI | 6 | ~~~VKLIH~ | 18 | No | No | 0.48 | −0.96 | −1.09 | −1.39 | −1.43 | −1.72 | −1.95 | −0.01 | −1.35 | 0.35 |
| FFVKLIHIP | 7 | ~~~KLIHI~ | 19 | No | Yes | −0.77 | −1.12 | −1.74 | −1.04 | −1.37 | −0.65 | −0.75 | −1.10 | −0.02 | −1.03 |
| FVKLIHIPI | 8 | ~~~LIHIP~ | 20 | No | No | −2.24 | −1.53 | −0.79 | −0.68 | −0.65 | −0.86 | −0.05 | 0.39 | −2.79 | 0.37 |
| VKLIHIPIN | 9 | ~~~IHIPI~ | 21 | No | No | −0.01 | 0.69 | 0.55 | 0.51 | 0.05 | 0.77 | 0.39 | 0.87 | 0.95 | 0.08 |
| KLIHIPINN | 10 | ~~~HIPIN~ | 22 | No | Yes | 0.22 | −1.01 | 0.25 | 0.38 | −0.30 | −0.21 | −0.37 | −0.63 | 1.20 | −1.14 |
| LIHIPINNI | 11 | ~~~IPINN~ | 23 | No | No | 0.95 | −1.02 | −0.75 | −1.58 | −0.87 | −2.17 | −1.55 | 0.46 | −1.30 | 2.37 |
| IHIPINNII | 12 | ~~~PINNI~ | 24 | No | No | 0.58 | −0.51 | −0.85 | −0.11 | −0.83 | 0.39 | 0.38 | 0.35 | −0.30 | 0.88 |
| | | | TCEM | | | | | | | | | | | | |
| | SEQ | | SEQ | | | Representative MHC I B alleles | | | | | | | | | |
| | ID | | ID | Cat | Cat | B_ | B_ | B_ | B_ | B_ | B_ | B_ | B_ | B_ | B_ |
| 9-mer | NO.: | TCEM 1 | NO.: | S | L | 0702 | 0801 | 0802 | 0803 | 1501 | 1502 | 1503 | 2703 | 2705 | 3501 |
| IMGFIGFFV | 1 | ~~~FIGFF~ | 13 | No | Yes | 0.38 | 0.50 | −0.99 | −1.05 | −0.62 | −0.32 | 0.37 | 0.83 | −0.05 | −0.04 |
| MGFIGFFVK | 2 | ~~~IGFFV~ | 14 | No | No | 0.52 | 0.93 | 1.25 | 1.27 | 0.64 | 1.26 | 2.68 | 0.09 | 0.09 | 1.35 |
| GFIGFFVKL | 3 | ~~~GFFVK~ | 15 | No | No | 0.83 | −0.62 | 0.70 | 0.07 | −0.42 | −0.78 | −0.36 | −0.53 | 0.13 | 0.25 |
| FIGFFVKLI | 4 | ~~~FFVKL~ | 16 | No | No | −0.56 | −1.14 | −0.34 | −1.64 | −0.44 | −1.50 | 0.16 | −0.03 | 0.36 | −0.95 |
| IGFFVKLIH | 5 | ~~~FVKLI~ | 17 | No | Yes | −0.48 | 0.65 | 0.51 | 0.68 | 0.17 | 0.67 | −0.48 | −1.53 | −0.20 | −1.52 |
| GFFVKLIHI | 6 | ~~~VKLIH~ | 18 | No | No | 2.19 | −1.49 | 0.11 | −0.17 | −0.41 | −0.05 | −1.13 | −0.18 | 0.17 | 0.45 |
| FFVKLIHIP | 7 | ~~~KLIHI~ | 19 | No | Yes | 0.03 | 0.17 | 0.48 | 0.91 | −0.31 | −0.40 | 0.35 | −0.56 | 0.24 | −0.10 |
| FVKLIHIPI | 8 | ~~~LIHIP~ | 20 | No | No | −1.85 | −1.73 | −1.17 | −2.14 | 1.20 | −1.31 | 0.95 | −0.66 | 0.26 | −0.87 |
| VKLIHIPIN | 9 | ~~~IHIPI~ | 21 | No | No | 0.92 | −0.13 | 0.74 | 0.89 | 0.85 | 0.90 | 0.23 | 0.45 | −1.49 | 0.76 |
| KLIHIPINN | 10 | ~~~HIPIN~ | 22 | No | Yes | 0.47 | 0.57 | −0.05 | 0.69 | 0.38 | 0.16 | 1.57 | −0.12 | −0.21 | 0.43 |
| LIHIPINNI | 11 | ~~~IPINN~ | 23 | No | No | −0.33 | −1.82 | 0.44 | −1.77 | −0.92 | −2.65 | 0.28 | −0.27 | 0.40 | −0.53 |
| IHIPINNII | 12 | ~~~PINNI~ | 24 | No | No | −0.17 | 0.19 | 0.38 | 0.58 | 0.00 | −1.45 | −0.98 | 0.01 | −0.29 | 0.17 |

#TCEM refers to T cell exposed motif-see definitions.
##CatS and CatL refer to whether the predicted probability of the peptide, as it occurs in the natural protein context
in vivo, being excised as a correctly sized peptide for binding in theMHC groove. A probability of over 50% is
indicated as yes, however lower probabilities are adequate to allow some presentation
###Predicted binding is shown as in standard deviation units. Binding predictions in icLN50 are calculated for each
allele for every sequential peptide in the protein of origin and standardized to a zero mean to provide an index of
competitive binding.Hence negative numbers indicate higher affinity binding.

TABLE 2
Predicted binding of peptides of SEC61G to representative DRB alleles (Footnotes as for Table 1)

| | SEQ | | SEQ | Cat | Cat | | | | | | | | | | | | |
|---|---|---|---|---|---|---|---|---|---|---|---|---|---|---|---|---|---|
| | ID | TCEM | ID | S | L | DRB1 | DRB1 | DRB1 | DRB1 | DRB1 | DRB1 | DRB1 | DRB1 | DRB1 | DRB1 | DRB3 | DRB3 |
| peptide | NO.: | IIA # | NO.: | ## | ## | 0101 | 0301 | 0401 | 0701 | 0801 | 0901 | 1101 | 1201 | 1301 | 1501 | 0101 | 0202 |
| IGFAIMGFIGFFVKL | 25 | IM~F~GF | 37 | No | No | −0.41 | −1.37 | 0.26 | −0.62 | −1.21 | −0.35 | −0.84 | −0.53 | −1.76 | −0.56 | −1.60 | −0.95 |
| GFAIMGFIGFFVKLI | 26 | MG~I~FF | 38 | Yes | Yes | −0.71 | −0.09 | −0.46 | −0.15 | −1.58 | −1.33 | −0.74 | −1.57 | −0.76 | −0.96 | −0.84 | −0.54 |
| FAIMGFIGFFVKLIH | 27 | GF~G~FV | 39 | Yes | Yes | −0.66 | −0.08 | −0.73 | −0.80 | −1.19 | −0.54 | −0.33 | −1.27 | −1.23 | −0.90 | −2.03 | −0.43 |
| AIMGFIGFFVKLIHI | 28 | FI~F~VK | 40 | Yes | Yes | −0.38 | −1.24 | −0.76 | −0.74 | −0.91 | −1.06 | −1.32 | −1.18 | −1.11 | −0.87 | −0.87 | −0.95 |
| IMGFIGFFVKLIHIP | 29 | IG~F~KL | 41 | Yes | Yes | −0.79 | −0.74 | −1.01 | −0.48 | −1.51 | −1.52 | −1.66 | −1.06 | −1.74 | −2.62 | −1.31 | −1.42 |
| MGFIGFFVKLIHIPI | 30 | GF~V~LI | 42 | Yes | No | −0.40 | −1.44 | −0.89 | −0.98 | −1.31 | −0.65 | −2.40 | −1.70 | −1.52 | −1.55 | −2.54 | −1.39 |
| GFIGFFVKLIHIPIN | 31 | FF~K~IH | 43 | No | No | −0.87 | −1.16 | −1.41 | −0.89 | −0.89 | −0.57 | −1.57 | −1.33 | −0.68 | −1.04 | −0.59 | −0.41 |
| FIGFFVKLIHIPINN | 32 | FV~L~HI | 44 | Yes | No | −0.68 | −0.90 | −1.38 | −0.7 | −1.30 | −1.00 | −0.88 | −0.25 | −2.52 | −1.84 | −1.01 | −2.68 |
| IGFFVKLIHIPINNI | 33 | VK~I~IP | 45 | Yes | No | −0.33 | −1.69 | −1.83 | −0.48 | −2.59 | −1.03 | −2.17 | −1.22 | −1.86 | −1.33 | −1.20 | −1.23 |
| GFFVKLIHIPINNII | 34 | KL~H~PI | 46 | | | −1.05 | −0.33 | −1.51 | −1.08 | −1.27 | −1.23 | −1.00 | −2.65 | −0.93 | −1.34 | −1.32 | −1.20 |
| FFVKLIHIPINNIIV | 35 | LI~I~IN | 47 | | | −1.02 | −1.32 | −2.62 | −2.67 | −1.76 | −1.64 | −1.39 | −1.06 | −0.90 | −1.53 | −0.82 | −0.77 |
| FVKLIHIPINNIIVG | 36 | IH~P~NN | 48 | | | −0.96 | −0.29 | −1.41 | −0.99 | −1.34 | −0.57 | −1.32 | −0.54 | −0.60 | −0.85 | 0.16 | −1.27 |

#Foot notes as for Table 1

TABLE 3
Peptides designed to optimize binding for specific MHC I alleles
of T cell exposed motifs found in SEC61G

| | | | | | | | |
|---|---|---|---|---|---|---|---|
| | | Predicted | | | Predicted | | |
| | | binding of | | | binding of | | |
| originating | SEQ | originating | proposed | SEQ | proposed | TCEM | SEQ |
| peptide | ID NO.: | peptide | peptide | ID NO.: | peptide | core | ID NO.: |

Peptides designed for optimal binding to A_1101

| | | | | | | | |
|---|---|---|---|---|---|---|---|
| IGFFVKLIH | 5 | −1.32 | SGRFVKLIK | 49 | −2.07 | FVKLI | 73 |
| IGFFVKLIH | 5 | −1.32 | TTGFVKLIR | 50 | −2.03 | FVKLI | 74 |
| IGFFVKLIH | 5 | −1.32 | DAEFVKLIK | 51 | −2.03 | FVKLI | 75 |
| IGFFVKLIH | 5 | −1.32 | GSNFVKLIR | 52 | −2.01 | FVKLI | 76 |
| GFIGFFVKL | 3 | −0.74 | SSNGFFVKR | 53 | −2.04 | GFFVK | 77 |
| GFIGFFVKL | 3 | −0.74 | RIEGFFVKR | 54 | −2.03 | GFFVK | 78 |
| GFIGFFVKL | 3 | −0.74 | SGYGFFVKR | 55 | −2.03 | GFFVK | 79 |
| GFIGFFVKL | 3 | −0.74 | KSAGFFVKP | 56 | −2.03 | GFFVK | 80 |
| FFVKLIHIP | 7 | −1.03 | SSVKLIHIP | 57 | −2.03 | KLIHI | 81 |
| FFVKLIHIP | 7 | −1.03 | EITKLIHIK | 58 | −2.03 | KLIHI | 82 |
| FFVKLIHIP | 7 | −1.03 | ESPKLIHIK | 59 | −2.02 | KLIHI | 83 |
| FFVKLIHIP | 7 | −1.03 | AIQKLIHIR | 60 | −2.02 | KLIHI | 84 |

Peptides designed for optimal binding to A_3201

| | | | | | | | |
|---|---|---|---|---|---|---|---|
| FIGFFVKLI | 4 | −0.38 | RQRFFVKLF | 61 | −2.13 | FFVKL | 85 |
| FIGFFVKLI | 4 | −0.38 | RKYFFVKLI | 62 | −2.08 | FFVKL | 86 |
| FIGFFVKLI | 4 | −0.38 | KGQFFVKLF | 63 | 2.04 | FFVKL | 87 |
| FIGFFVKLI | 4 | −0.38 | RTQFFVKLI | 64 | −1.99 | FFVKL | 88 |
| GFIGFFVKL | 3 | −1.60 | KNAGFFVKF | 65 | −2.05 | GFFVK | 89 |
| GFIGFFVKL | 3 | −1.60 | KGAGFFVKI | 66 | −2.02 | GFFVK | 90 |
| GFIGFFVKL | 3 | −1.60 | KARGFFVKW | 67 | −2.01 | GFFVK | 91 |
| GFIGFFVKL | 3 | −1.60 | EAQGFFVKF | 68 | 2.01 | GFFVK | 92 |
| GFFVKLIHI | 6 | −1.10 | RAMVKLIHL | 69 | 2.07 | VKLIH | 93 |
| GFFVKLIHI | 6 | −1.10 | KGFVKLIHF | 70 | −2.06 | VKLIH | 94 |
| GFFVKLIHI | 6 | −1.10 | RMMVKLIHL | 71 | 2.00 | VKLIH | 95 |
| GFFVKLIHI | 6 | −1.10 | RPMVKLIHF | 72 | 2.00 | VKLIH | 96 |

TABLE 4
Predicted binding of peptides of VOPP to representative MHC I A and MHC I B alleles

| | SEQ | | SEQ | | | Predicted binding to representative MCH I A alleles | | | | | | | | | |
|---|---|---|---|---|---|---|---|---|---|---|---|---|---|---|---|
| | ID | | ID | Cat | Cat | A_ | A_ | A_ | A_ | A_ | A_ | A_ | A_ | A_ | A_ |
| 9-mer | NO.: | TEMI # | NO.: | S | L | 0101 | 0201 | 0202 | 0203 | 0206 | 0211 | 0212 | 0301 | 0801 | 1101 |
| HCWYFEGLY | 97 | ~~~YFEGL~ | 127 | No | No | −0.91 | −0.07 | 0.85 | 0.82 | 0.90 | 0.18 | 1.16 | −1.96 | −0.51 | −0.63 |
| CWYFEGLYP | 98 | ~~~FEGLY~ | 128 | No | No | 0.86 | 1.47 | −0.66 | −0.46 | 0.09 | −0.07 | 0.54 | −1.54 | −1.25 | −0.87 |
| WYFEGLYPT | 99 | ~~~EGLYP~ | 129 | No | No | −0.45 | −1.36 | −1.86 | −1.77 | −2.46 | −1.31 | −1.16 | 0.13 | −0.27 | −0.03 |
| YFEGLYPTY | 100 | ~~~GLYPT~ | 130 | No | No | −1.65 | −0.53 | −1.32 | −0.71 | −0.21 | −0.20 | 0.19 | 0.03 | 0.41 | 0.21 |
| FWFLLMMGV | 101 | ~~~LLMMG~ | 131 | No | No | 0.36 | −1.54 | −2.31 | −2.34 | −2.08 | −2.11 | −2.07 | −0.21 | −2.71 | 0.48 |
| WFLLMMGVL | 102 | ~~~LMMGV~ | 132 | No | No | 0.21 | −1.12 | −2.36 | −2.20 | −1.74 | −1.62 | −1.87 | −0.05 | −2.17 | 1.02 |
| FLLMMGVLF | 103 | ~~~MMGVL~ | 133 | Yes | No | −0.89 | −2.51 | −2.45 | −1.79 | −1.21 | −0.89 | −1.35 | −0.11 | −0.67 | −0.47 |
| LLMMGVLFC | 104 | ~~~MGVLF~ | 134 | No | Yes | −0.14 | −1.48 | −1.36 | −1.53 | −1.61 | −1.67 | −1.84 | −0.46 | −0.79 | 0.32 |
| LMMGVLFCC | 105 | ~~~GVLFC~ | 135 | No | No | −0.04 | −2.25 | −1.12 | −1.57 | −1.83 | −2.12 | −2.00 | −0.12 | −0.28 | 0.16 |
| MMGVLFCCG | 106 | ~~~VLFCC~ | 136 | Yes | Yes | −0.67 | −1.88 | −1.25 | −1.36 | −1.11 | −2.07 | −1.80 | −0.26 | −0.63 | 0.32 |
| MGVLFCCGA | 107 | ~~~LFCCG~ | 137 | No | Yes | −0.72 | −0.95 | −0.55 | −0.77 | −0.99 | −0.95 | −0.03 | 0.99 | −1.20 | 0.65 |
| GVLFCCGAG | 108 | ~~~FCCGA~ | 138 | No | No | 0.43 | −0.84 | −0.48 | −1.04 | −0.91 | −0.91 | −1.70 | 0.81 | −0.89 | 0.74 |
| VLFCCGAGF | 109 | ~~~CCGAG~ | 139 | No | No | 0.28 | −1.90 | −1.43 | −1.66 | −0.93 | −1.56 | −2.02 | −0.54 | −0.89 | −0.04 |
| LFCCGAGFF | 110 | ~~~CGAGF~ | 140 | No | No | −0.59 | −0.04 | −1.70 | −1.64 | −0.99 | −1.60 | −1.86 | −0.36 | −0.05 | 0.39 |
| FCCGAGFFI | 111 | ~~~GAGFF~ | 141 | No | No | −2.00 | −2.17 | −1.37 | −0.56 | −1.49 | −1.95 | −1.56 | 0.34 | 0.24 | 0.18 |
| | SEQ | | SEQ | | | Predicted binding to representative MHC I B alleles | | | | | | | | | |
| | ID | | ID | Cat | Cat | B_ | B_ | B_ | B_ | B_ | B_ | B_ | B_ | B_ | B_ |
| 9-mer | NO.: | TEMI # | NO.: | S | L | 0702 | 0801 | 0802 | 0803 | 1501 | 1502 | 1503 | 2703 | 2705 | 3501 |
| HQWYFEGLY | 112 | ~~~YFEGL~ | 142 | No | No | −0.36 | 0.20 | −0.22 | −0.73 | −0.98 | −1.49 | −1.94 | 0.18 | −0.45 | −0.92 |
| CWYFEGLYP | 113 | ~~~FEGLY~ | 143 | No | No | 1.12 | −0.73 | −0.41 | 1.51 | 1.65 | −0.57 | −1.00 | 0.19 | 0.38 | 0.33 |
| WYFEGLYPT | 114 | ~~~EGLYP~ | 144 | No | No | 0.12 | −0.50 | 0.01 | 0.06 | −0.24 | −1.02 | −0.67 | −0.05 | −2.16 | −0.45 |
| YFEGLYPTY | 115 | ~~~GLYPT~ | 145 | No | No | 0.26 | −0.18 | −0.47 | −0.83 | −1.76 | −2.61 | −1.57 | −0.15 | −0.37 | −1.34 |
| FWFLLMMGV | 116 | ~~~LLMMG~ | 146 | No | No | 0.16 | −1.75 | −0.32 | −1.23 | −0.26 | −1.73 | −0.82 | −0.17 | −1.88 | −0.17 |
| WFLLMMGVL | 117 | ~~~LMMGV~ | 147 | No | No | −0.38 | −2.17 | −0.68 | −1.49 | −1.11 | −1.08 | −0.51 | −0.07 | −0.38 | −1.18 |
| FLLMMGVLF | 118 | ~~~MMGVL~ | 148 | Yes | No | −0.11 | −1.17 | −0.98 | −0.68 | −2.43 | −1.56 | −1.52 | −0.22 | −0.39 | −1.95 |
| LLMMGVLFC | 119 | ~~~MGVLF~ | 149 | No | Yes | −0.05 | −0.71 | 0.40 | −0.43 | −0.34 | −1.33 | −1.12 | 0.22 | 0.21 | −0.81 |
| LMMGVLFCC | 120 | ~~~GVLFC~ | 150 | No | No | 0.55 | −0.40 | 1.45 | 0.78 | −1.21 | −0.28 | −0.82 | 0.42 | 0.05 | −0.33 |
| MMGVLFCCG | 121 | ~~~VLFCC~ | 151 | Yes | Yes | 1.01 | −0.74 | 0.28 | 0.03 | −0.65 | −0.21 | 2.55 | −0.17 | 0.20 | 0.66 |
| MGVLFCCGA | 122 | ~~~LFCCG~ | 152 | No | Yes | −0.39 | −0.09 | −0.08 | 0.19 | −0.51 | −0.42 | 1.25 | 0.05 | 0.43 | −1.00 |
| GVLFCCGAG | 123 | ~~~FCCGA~ | 153 | No | No | −0.83 | −0.15 | −0.90 | −0.89 | −0.75 | 0.45 | 1.24 | −0.51 | 0.26 | −0.01 |
| VLFCCGAGF | 124 | ~~~CCGAG~ | 154 | No | No | −0.99 | −1.22 | −0.52 | −0.30 | −2.63 | −1.21 | −1.37 | −0.08 | −0.12 | −1.78 |
| LFCCGAGFF | 125 | ~~~CGAGF~ | 155 | No | No | 0.48 | 0.95 | 0.65 | 0.63 | −1.91 | −2.82 | −1.92 | 0.06 | 0.36 | −1.82 |
| FCCGAGFFI | 126 | ~~~GAGFF~ | 156 | No | No | 0.04 | 0.45 | 1.34 | 0.94 | −1.27 | −1.94 | −0.89 | −0.12 | 0.57 | −1.30 |

#Footnotes as for Table 1

TABLE 5
Predicted binding of peptides of VOPP to representative DRB alleles

| | SEQ | SEQ | | | | | | | | | | | | | | |
|---|---|---|---|---|---|---|---|---|---|---|---|---|---|---|---|---|
| | ID | ID | Cat | Cat | DRB1 | DRB1 | DRB1 | DRB1 | DRB1 | DRB1 | DRB1 | DRB1 | DRB1 | DRB1 | DRB3 | DRB3 |
| peptide | NO.: | NO.: | S | L | 0101 | 0301 | 0401 | 0701 | 0801 | 0901 | 1101 | 1201 | 1301 | 1501 | 0202 | 0301 |

| | | | | | | | | | | | | | | | | |
|---|---|---|---|---|---|---|---|---|---|---|---|---|---|---|---|---|
| KKHCWYFEGLYPTYY | 157 | 170 | Yes | Yes | −0.97 | −1.21 | −1.71 | −1.31 | −0.48: | −1.87 | −1.97 | −0.85 | −0.79 | −1.69 | −0.90 | −0.24 |
| KHCWYFEGLYPTYYI | 158 | 171 | Yes | Yes | −2.73 | −0.99 | −2.21 | −1.87 | −1.07 | −0.95 | −0.99 | −1.04 | −1.56 | −2.00 | −1.52 | −0.83 |
| HCWYFEGLYPTYYIC | 159 | 172 | Non | Non | −2.18 | −1.26 | −1.48 | −1.40 | −1.25 | −1.77 | −1.08 | −0.90 | −1.40 | −1.53 | −1.67 | −1.83 |
| RALSIQRLWYFWFLL | 160 | 173 | Non | Yes | 0.14 | −1.04 | −0.29 | −0.93 | −1.43 | −0.40 | −1.01 | −0.79 | −2.14 | −2.62 | −1.20 | −1.70 |
| ALSIQRLWYFWFLLM | 161 | 174 | Non | Yes | −0.18 | −0.91 | −0.68 | −1.87 | −1.86 | −1.02 | −1.25 | −2.14 | −2.06 | −2.64 | −1.09 | −1.94 |
| LSIQRLWYFWFLLMM | 162 | 175 | Non | Yes | 0.07 | −0.56 | −1.17 | −1.16 | −1.93 | −1.53 | −1.32 | −2.86 | −2.50 | −1.35 | −1.57 | −1.76 |
| SIQRLWYFWFLLMMG | 163 | 176 | Non | Yes | −0.60 | −0.92 | −1.44 | −0.89 | −1.79 | −1.18 | −1.91 | −1.54 | −1.63 | −1.31 | −1.77 | −1.39 |
| IQRLWYFWFLLMMGV | 164 | 177 | Non | Yes | −0.49 | −0.94 | −1.64 | −1.00 | −2.59 | −1.73 | −1.93 | −2.29 | −2.66 | −0.95 | −2.21 | −1.95 |
| QRLWYFWFLLMMGVL | 165 | 178 | Non | Non | −0.78 | −0.75 | −1.93 | −1.38 | −2.16 | −1.22 | −2.83 | −2.11 | −1.81 | −1.24 | −2.24 | −2.22 |
| RLWYFWFLLMMGVLF | 166 | 179 | Non | Non | −0.98 | −0.97 | −1.96 | −2.65 | −2.35 | −1.92 | −2.63 | −2.05 | −2.02 | −1.19 | −2.48 | −2.38 |
| LWYFWFLLMMGVLFC | 167 | 180 | Non | Non | −1.70 | −1.27 | −2.86 | −2.36 | −2.54 | −1.49 | −1.86 | −1.79 | −1.36 | −1.01 | −1.93 | −2.16 |
| WYFWFLLMMGVLFCC | 168 | 181 | Non | Non | −1.86 | −1.56 | −2.23 | −2.34 | −1.87 | −1.57 | −1.49 | −1.26 | −0.39 | −1.50 | −2.22 | −1.84 |
| YFWFLLMMGVLFCCG | 169 | 182 | Non | Non | −0.88 | −1.60 | −1.24 | −1.44 | −1.64 | −2.87 | −1.12 | −1.84 | −0.75 | −1.14 | −2.30 | −1.89 |

TABLE 6
Peptides designed to optimize binding for specific MHC I alleles
of T cell exposed motifs found in VOPP

| | | | | | | | |
|---|---|---|---|---|---|---|---|
| | | Predicted | | | Predicted | | |
| | SEQ | binding of | | SEQ | binding of | | SEQ |
| originating | ID | originating | proposed | ID | proposed | TCEM | ID |
| peptide | NO.: | peptide | peptide | NO.: | peptide | core | NO.: |

Peptides designed for A_1101

| | | | | | | | |
|---|---|---|---|---|---|---|---|
| CWYFEGLYP | 113 | −0.87 | GGGFEGLYK | 183 | −2.05 | FEGLY | 203 |
| CWYFEGLYP | 113 | −0.87 | ESHFEGLYK | 184 | −2.05 | FEGLY | 203 |
| CWYFEGLYP | 113 | −0.87 | KQWFEGLYR | 185 | −2.04 | FEGLY | 203 |
| CWYFEGLYP | 113 | −0.87 | KNLFEGLYR | 186 | −2.04 | FEGLY | 203 |
| FLLMMGVLF | 118 | −0.47 | RNMMMGVLR | 187 | −2.08 | MMGVL | 204 |
| FLLMMGVLF | 118 | −0.47 | DAKMMGVLK | 188 | 2.07 | MMGVL | 204 |
| FLLMMGVLF | 118 | −0.47 | RQPMMGVLK | 189 | −2.04 | MMGVL | 204 |
| FLLMMGVLF | 118 | −0.47 | NGSMMGVLK | 190 | −2.03 | MMGVL | 204 |
| HCWYFEGLY | 112 | −0.63 | RVVYFEGLR | 191 | −2.14 | YFEGL | 205 |
| HCWYFEGLY | 112 | −0.63 | GAEYFEGLK | 192 | −2.14 | YFEGL | 205 |
| HCWYFEGLY | 112 | −0.63 | GDMYFEGLK | 193 | −2.09 | YFEGL | 205 |
| HCWYFEGLY | 112 | −0.63 | SNLYFEGLR | 194 | −2.04 | YFEGL | 205 |
| HCWYFEGLY | 112 | −0.63 | PIRYFEGLK | 195 | −2.04 | YFEGL | 205 |
| HCWYFEGLY | 112 | −0.63 | AGQYFEGLK | 196 | −2.03 | YFEGL | 205 |

Peptides designed for A_3001

| | | | | | | | |
|---|---|---|---|---|---|---|---|
| GVLFCCGAG | 123 | −0.34 | KVRFCCGAA | 197 | −1.97 | FCCGA | 206 |
| GVLFCCGAG | 123 | −0.34 | RGRFCCGAS | 198 | −1.95 | FCCGA | 206 |
| GVLFCCGAG | 123 | −0.34 | KVKFCCGAG | 199 | −1.92 | FCCGA | 206 |
| HCWYFEGLY | 112 | −0.40 | KGPYFEGLS | 200 | −2.05 | YFEGL | 205 |
| HCWYFEGLY | 112 | −0.40 | RDKYFEGLG | 201 | −2.04 | YFEGL | 205 |
| HCWYFEGLY | 112 | −0.40 | TNRYFEGLG | 202 | −2.03 | YFEGL | 205 |

TABLE 7
Predicted binding of peptides of LANC2 to representative MHC I A and MHC I B alleles

| | SEQ | Cat | Cat | | SEQ | Predicted binding to representative MHC I A alleless | | | | | | | | | |
|---|---|---|---|---|---|---|---|---|---|---|---|---|---|---|---|
| | ID | S | L | | ID | | | | | | | | | | A_ |
| 9-mer | NO.: | # | 3 | TCEM I | NO: | A0101 | A0201 | A0202 | A0203 | A0206 | A0211 | A0212 | A0301 | A0801 | 1101 |
| AYTGWTGIA | 206 | No | Yes | ~~~GWTGI~ | 257 | 0.15 | −0.03 | −1.81 | −2.30 | −1.07 | −1.15 | −1.79 | 0.66 | −0.36 | 0.27 |
| YTGWTGIAL | 207 | No | No | ~~~WTGIA~ | 258 | −1.44 | −1.55 | −0.86 | −0.18 | −1.01 | −0.22 | −0.02 | 0.77 | −0.88 | −0.08 |
| TGWTGIALL | 208 | No | No | ~~~TGIAL~ | 259 | −0.19 | −1.12 | −0.67 | −0.79 | −0.70 | −0.55 | 0.36 | −1.19 | −0.56 | −0.93 |
| GWTGIALLY | 209 | No | Yes | ~~~GIALL~ | 260 | −0.01 | 1.66 | −1.54 | −0.69 | −0.82 | 0.28 | 0.02 | −0.17 | 0.89 | −1.00 |
| WTGIALLYL | 210 | No | No | ~~~IALLY~ | 261 | −1.94 | −2.38 | −1.79 | −1.35 | −2.53 | −0.55 | 0.07 | 0.72 | −0.49 | −0.21 |
| TGIALLYLQ | 211 | No | No | ~~~ALLYL~ | 262 | −0.54 | −0.04 | 0.40 | 0.89 | −0.54 | 0.18 | 1.49 | −1.61 | 1.35 | −1.83 |
| GIALLYLQL | 212 | No | No | ~~~LLYLQ~ | 263 | 0.07 | −1.86 | −1.57 | −1.22 | −0.99 | −1.20 | −1.53 | −0.61 | −0.79 | −0.78 |
| IALLYLQLY | 213 | No | No | ~~~LYLQL~ | 264 | −1.50 | −0.08 | −0.13 | 0.33 | −0.51 | 0.94 | 0.69 | −1.95 | 0.05 | −1.93 |
| ALLYLQLYR | 214 | No | No | ~~~YLQLY~ | 265 | 0.38 | 0.05 | 0.20 | 1.54 | 0.62 | 0.50 | 0.75 | −2.50 | 1.20 | −2.38 |
| LLYLQLYRV | 215 | No | No | ~~~LQLYR~ | 266 | −0.35 | −3.30 | −1.91 | −2.12 | −2.52 | −2.06 | −2.26 | 0.00 | −1.87 | 0.31 |
| DLPDELLYG | 216 | No | Yes | ~~~DELLY~ | 267 | −0.16 | −2.45 | −1.45 | −2.08 | −2.06 | −2.02 | −2.09 | 0.79 | −0.56 | 0.24 |
| LPDELLYGR | 217 | No | No | ~~~ELLYG~ | 268 | −2.04 | 0.23 | 1.15 | 0.55 | 0.10 | 0.86 | 0.63 | −1.06 | 0.97 | −1.02 |
| PDELLYGRA | 218 | No | No | ~~~LLYGR~ | 269 | −0.76 | 1.71 | −0.54 | −0.09 | −0.41 | 0.68 | 0.42 | 0.94 | −0.19 | 0.87 |
| DELLYGRAG | 219 | No | No | ~~~LYGRA~ | 270 | 0.45 | 0.88 | 0.24 | −0.05 | −0.40 | −0.07 | −0.25 | 0.49 | −0.55 | 0.66 |
| ELLYGRAGY | 220 | No | No | ~~~YGRAG~ | 271 | 0.70 | 0.22 | 0.25 | 0.53 | 0.94 | 0.48 | 0.45 | −0.84 | −0.58 | −0.36 |
| LLYGRAGYL | 221 | No | No | ~~~GRAGY~ | 272 | −0.20 | −2.68 | −2.41 | −2.42 | −1.05 | −1.53 | −1.92 | −0.55 | −1.52 | 0.31 |
| ALLYLNTEI | 222 | No | No | ~~~YLNTE~ | 273 | 1.60 | −2.56 | −1.26 | −1.52 | −0.91 | −1.10 | −1.40 | 0.75 | −1.07 | 0.76 |
| LLYLNTEIG | 223 | No | No | ~~~LNTEI~ | 274 | −0.38 | −1.93 | −1.54 | −2.02 | −1.43 | −2.12 | −2.09 | −0.03 | −0.77 | 0.36 |
| LYLNTEIGP | 224 | No | No | ~~~NTEIG~ | 275 | 1.32 | 0.30 | −1.09 | −0.72 | −1.30 | 0.01 | −0.31 | −0.42 | −0.69 | −0.49 |
| YLNTEIGPG | 225 | No | No | ~~~TEIGP~ | 276 | −0.29 | −2.36 | −2.57 | −2.64 | −2.25 | −2.03 | −2.03 | 0.46 | −0.88 | 0.22 |
| HGMAGIYYM | 226 | No | No | ~~~AGIYY~ | 277 | −0.86 | −0.74 | 0.04 | 0.02 | −1.06 | 0.07 | 0.19 | −0.39 | −0.67 | −0.47 |
| GMAGIYYML | 227 | No | No | ~~~GIYYM~ | 278 | 0.03 | −3.03 | −1.80 | −1.25 | −1.49 | −1.49 | −1.67 | −0.16 | 0.07 | −0.09 |
| MAGIYYMLM | 228 | No | No | ~~~IYYML~ | 279 | −1.94 | −0.96 | −0.48 | −0.29 | −0.96 | −0.11 | 0.37 | −0.98 | −1.11 | −1.07 |
| AGIYYMLMQ | 229 | No | Yes | ~~~YYMLM~ | 280 | −0.12 | 0.06 | 0.89 | 1.42 | −0.16 | 0.71 | 0.28 | −1.75 | 0.58 | −1.94 |
| GIYYMLMQP | 230 | No | No | ~~~YMLMQ~ | 281 | −0.64 | −1.17 | −0.56 | −0.73 | −0.72 | −1.58 | −1.57 | −2.12 | −0.69 | −2.28 |
| IYYMLMQPA | 231 | No | No | ~~~MLMQP~ | 282 | −0.08 | −1.25 | −2.21 | −1.95 | −1.93 | −1.78 | −1.77 | 0.16 | −1.57 | 0.41 |
| YYMLMQPAA | 232 | No | No | ~~~LMQPA~ | 283 | 0.45 | −1.20 | −1.89 | −1.76 | −1.75 | −1.23 | −1.53 | 0.40 | −1.04 | 0.39 |
| YMLMQPAAK | 233 | No | No | ~~~MQPAA~ | 284 | 0.34 | −0.65 | 0.45 | 0.24 | 0.36 | −0.09 | 0.06 | −2.60 | 0.71 | −2.27 |
| MLMQPAAKV | 234 | No | No | ~~~QPAAK~ | 285 | 0.29 | −2.91 | −1.99 | −2.25 | −1.81 | −1.75 | −1.86 | −0.16 | −1.83 | 0.07 |
| RLVHWCHGA | 235 | No | No | ~~~HWCHG~ | 286 | 0.33 | −2.54 | −1.92 | −1.63 | −1.67 | −1.79 | −2.14 | −0.02 | −2.71 | 0.31 |
| LVHWCHGAP | 236 | No | No | ~~~WCHGA~ | 287 | −0.80 | 0.07 | −0.06 | −0.08 | 0.21 | −0.40 | −0.39 | −1.92 | −0.25 | −1.54 |
| VHWCHGAPG | 237 | No | No | ~~~CHGAP~ | 288 | −0.04 | −0.48 | −0.85 | −1.59 | −1.01 | −1.51 | −1.83 | 0.23 | −0.59 | −0.12 |
| HWCHGAPGV | 238 | No | No | ~~~HGAPG~ | 289 | 2.38 | 0.24 | −1.98 | −1.66 | −1.84 | −1.18 | −1.77 | 0.01 | −0.95 | 0.57 |
| PGVIHMLMQ | 239 | No | No | ~~~IHMLM~ | 290 | −0.74 | 0.32 | 0.58 | 0.67 | −0.68 | 0.25 | 0.48 | −1.37 | 1.12 | −1.56 |
| GVIHMLMQA | 240 | No | No | ~~~HMLMQ~ | 291 | −0.28 | −2.25 | −0.88 | −1.49 | −2.09 | −2.38 | −2.16 | 0.32 | −0.46 | −0.76 |
| VIHMLMQAY | 241 | No | No | ~~~MLMQA~ | 292 | −2.46 | 0.26 | −0.54 | 0.00 | 0.54 | −0.49 | 0.20 | −1.53 | −0.67 | −1.41 |
| IHMLMQAYK | 242 | No | No | ~~~LMQAY~ | 293 | 0.20 | 0.25 | 0.48 | 1.24 | 0.62 | 0.79 | 0.85 | −2.36 | 0.93 | −2.17 |
| RIPDRPYSL | 243 | No | No | ~~~DRPYS~ | 294 | 1.14 | −1.83 | −1.46 | −1.35 | −1.01 | −1.02 | −1.64 | −1.07 | −3.18 | 0.24 |
| IPDRPYSLF | 244 | No | Yes | ~~~RPYSL~ | 295 | −2.40 | 0.10 | −1.24 | −0.64 | −0.62 | 0.70 | 0.35 | 0.18 | −0.91 | 0.14 |
| PDRPYSLFE | 245 | No | No | ~~~PYSLF~ | 296 | −0.49 | 1.61 | 1.00 | 0.85 | 0.84 | 0.71 | 0.71 | 0.12 | 0.43 | −0.44 |
| DRPYSLFEG | 246 | No | No | ~~~YSLFE~ | 297 | 0.61 | 1.37 | −0.44 | −0.51 | −0.93 | 0.26 | 1.09 | 0.35 | −0.61 | 0.50 |
| RPYSLFEGM | 247 | No | No | ~~~SLFEG~ | 298 | 0.73 | −0.19 | −0.61 | −0.66 | −0.99 | 0.09 | 0.23 | −0.26 | −0.90 | −0.03 |
| PYSLFEGMA | 248 | No | Yes | ~~~LFEGM~ | 299 | 0.34 | 4.22 | −1.69 | −1.49 | −0.47 | −0.87 | −1.27 | 0.53 | −0.38 | 1.03 |
| YSLFEGMAG | 249 | No | No | ~~~FEGMA~ | 300 | −0.43 | −1.11 | −0.13 | −0.81 | −1.44 | −0.78 | −0.44 | 0.84 | −1.34 | −0.11 |
| SLFEGMAGA | 250 | No | No | ~~~EGMAG~ | 301 | 1.24 | −2.72 | −1.34 | −2.13 | −1.73 | −2.12 | −2.27 | −0.15 | −1.00 | 0.05 |
| LFEGMAGAI | 251 | No | No | ~~~GMAGA~ | 302 | −0.06 | −1.04 | −2.55 | −2.35 | −1.77 | −1.88 | −2.15 | 0.32 | −0.75 | 1.09 |
| FEGMAGAIH | 252 | No | No | ~~~MAGAI~ | 303 | −1.46 | 0.26 | 0.18 | 0.32 | 0.19 | 0.31 | 1.07 | −0.64 | 1.73 | 0.33 |
| EGMAGAIHF | 253 | No | Yes | ~~~AGAIH~ | 304 | 0.30 | 0.16 | 0.15 | 0.66 | 0.16 | 0.29 | 0.72 | −0.04 | −0.17 | 0.13 |
| GMAGAIHFL | 254 | No | Yes | ~~~GAIHF~ | 305 | 0.20 | −2.69 | −2.16 | −1.72 | −1.01 | −1.48 | −1.70 | −0.20 | 0.55 | 0.26 |
| MAGAIHFLS | 255 | No | No | ~~~AIHFL~ | 306 | −1.31 | −0.76 | −0.36 | −0.23 | −0.66 | −0.59 | 0.56 | −1.01 | −0.55 | −1.15 |
| AGAIHFLSD | 256 | No | No | ~~~IHFLS~ | 307 | −0.47 | 0.30 | 2.11 | 0.58 | −0.29 | 0.33 | 0.36 | −1.56 | 0.76 | −1.22 |
| | SEQ | | | | SEQ | | | | | | | | | | |
| | ID | Cat | Cat | | ID | Predicted binding to representative MHC I B alleles | | | | | | | | | |
| 9-mer | NO: | S | L | TCEM I | NO: | B0702 | B0801 | B0802 | B1501 | B1502 | B1503 | B2703 | B2705 | B3501 | |
| AYTGWTGIA | 206 | No | Yes | ~~~GWTGI~ | 257 | 2.16 | −0.93 | 0.42 | 0.15 | −0.57 | −0.31 | −0.19 | 0.80 | 1.02 | |
| YTGWTGIAL | 207 | No | No | ~~~WTGIA~ | 258 | −1.74 | −0.91 | −0.60 | −1.06 | −0.55 | −1.13 | 0.16 | 1.04 | −1.55 | |
| TGWTGIALL | 208 | No | No | ~~~TGIAL~ | 259 | −1.77 | −0.36 | 1.80 | −1.62 | −1.60 | −1.08 | −0.27 | −0.44 | −1.07 | |
| GWTGIALLY | 209 | No | Yes | ~~~GIALL~ | 260 | 0.60 | 0.96 | 0.06 | −0.03 | −0.59 | −1.74 | 0.12 | −0.84 | −0.66 | |
| WTGIALLYL | 210 | No | No | ~~~IALLY~ | 261 | −0.71 | −0.51 | 0.70 | −1.24 | −0.48 | −0.38 | −0.03 | −0.13 | −2.21 | |
| TGIALLYLQ | 211 | No | No | ~~~ALLYL~ | 262 | 1.15 | 0.47 | 3.11 | 0.53 | −0.02 | 1.04 | 0.23 | −0.13 | −0.52 | |
| GIALLYLQL | 212 | No | No | ~~~LLYLQ~ | 263 | −0.57 | −0.65 | −0.38 | −0.19 | 0.13 | 1.25 | −0.24 | 0.88 | −0.17 | |
| IALLYLQLY | 213 | No | No | ~~~LYLQL~ | 264 | 0.03 | −0.20 | 0.11 | −1.80 | −1.14 | 0.07 | 0.25 | −0.05 | −2.55 | |
| ALLYLQLYR | 214 | No | No | ~~~YLQLY~ | 265 | 0.54 | 0.79 | −0.15 | 0.28 | 1.16 | −0.58 | −0.24 | −0.07 | −0.19 | |
| LLYLQLYRV | 215 | No | No | ~~~LQLYR~ | 266 | 2.53 | −2.06 | −0.58 | 0.01 | −0.21 | −0.01 | −0.37 | 0.19 | 0.23 | |
| DLPDELLYG | 216 | No | Yes | ~~~DELLY~ | 267 | 0.71 | −1.54 | −0.38 | −0.34 | 1.27 | −0.26 | −0.30 | 0.66 | 0.66 | |
| LPDELLYGR | 217 | No | No | ~~~ELLYG~ | 268 | −0.66 | 1.31 | 0.01 | 1.05 | 1.43 | −1.29 | 0.28 | −0.41 | −1.71 | |
| PDELLYGRA | 218 | No | No | ~~~LLYGR~ | 269 | 0.99 | −0.53 | −0.47 | −0.31 | −0.08 | 1.10 | 0.61 | −0.09 | 1.75 | |
| DELLYGRAG | 219 | No | No | ~~~LYGRA~ | 270 | 0.19 | 0.10 | −0.19 | −0.05 | −0.77 | −0.40 | −0.17 | −0.07 | 1.38 | |
| ELLYGRAGY | 220 | No | No | ~~~YGRAG~ | 271 | −0.80 | −0.39 | −0.91 | −2.50 | −2.51 | −1.33 | −0.06 | −0.26 | −1.86 | |
| LLYGRAGYL | 221 | No | No | ~~~GRAGY~ | 272 | −1.19 | −2.48 | −2.09 | −1.47 | −1.25 | −0.46 | −0.22 | 0.82 | −0.67 | |
| ALLYLNTEI | 222 | No | No | ~~~YLNTE~ | 273 | −0.29 | −0.21 | −0.62 | −0.85 | −0.55 | 0.05 | −0.11 | 0.59 | −0.25 | |
| LLYLNTEIG | 223 | No | No | ~~~LNTEI~ | 274 | 0.03 | −1.04 | 0.29 | −0.60 | −0.60 | 1.07 | −0.05 | 0.25 | 0.32 | |
| LYLNTEIGP | 224 | No | No | ~~~NTEIG~ | 275 | −0.44 | 0.24 | −0.99 | −0.29 | −0.91 | −0.16 | 0.22 | −1.84 | −0.78 | |
| YLNTEIGPG | 225 | No | No | ~~~TEIGP~ | 276 | −0.72 | −1.91 | 0.19 | −0.83 | 0.08 | 1.13 | −0.34 | 0.88 | −0.24 | |
| HGMAGIYYM | 226 | No | No | ~~~AGIYY~ | 277 | −0.24 | 0.58 | 1.91 | −1.98 | −0.64 | −0.09 | −0.59 | −0.24 | −2.62 | |
| GMAGIYYML | 227 | No | No | ~~~GIYYM~ | 278 | 0.14 | −0.21 | 0.60 | −0.81 | 0.10 | −0.14 | −0.33 | −0.05 | 0.04 | |
| MAGIYYMLM | 228 | No | No | ~~~IYYML~ | 279 | −0.79 | −1.56 | 0.32 | −1.85 | −0.40 | 0.70 | −0.63 | −0.12 | −2.48 | |
| AGIYYMLMQ | 229 | No | Yes | ~~~YYMLM~ | 280 | 0.53 | −0.04 | 0.56 | 0.77 | 1.55 | 0.68 | 0.18 | 0.66 | −0.12 | |
| GIYYMLMQP | 230 | No | No | ~~~YMLMQ~ | 281 | 0.15 | −0.49 | 1.40 | 0.22 | 1.28 | 1.61 | −0.42 | 0.51 | 0.89 | |
| IYYMLMQPA | 231 | No | No | ~~~MLMQP~ | 282 | 1.24 | −1.20 | −0.36 | −0.13 | −1.19 | −0.03 | 0.13 | −0.39 | −0.07 | |
| YYMLMQPAA | 232 | No | No | ~~~LMQPA~ | 283 | −0.75 | −1.58 | −0.53 | −0.21 | −1.75 | −0.40 | 0.10 | −1.54 | −0.81 | |
| YMLMQPAAK | 233 | No | No | ~~~MQPAA~ | 284 | 0.23 | 0.04 | 0.48 | −0.51 | −0.70 | −0.34 | 0.04 | −1.31 | 0.11 | |
| MLMQPAAKV | 234 | No | No | ~~~QPAAK~ | 285 | −0.63 | −1.69 | −0.25 | −0.39 | −3.12 | −0.35 | 0.13 | 0.26 | −0.76 | |
| RLVHWCHGA | 235 | No | No | ~~~HWCHG~ | 286 | −0.54 | −1.27 | 0.73 | −0.80 | −1.29 | 0.07 | −0.30 | 0.29 | −0.87 | |
| LVHWCHGAP | 236 | No | No | ~~~WCHGA~ | 287 | −1.58 | −0.54 | −0.35 | −1.16 | 0.21 | 0.73 | 0.36 | 0.28 | −0.68 | |
| VHWCHGAPG | 237 | No | No | ~~~CHGAP~ | 288 | 0.34 | −0.63 | −0.64 | −0.82 | −1.12 | −0.53 | 0.25 | −0.99 | 0.77 | |
| HWCHGAPGV | 238 | No | No | ~~~HGAPG~ | 289 | 1.27 | −0.65 | 0.07 | 1.55 | −2.30 | −1.00 | −0.70 | 0.47 | −0.88 | |
| PGVIHMLMQ | 239 | No | No | ~~~IHMLM~ | 290 | 1.77 | 0.75 | 1.03 | 0.21 | 2.33 | 1.18 | −0.44 | 1.30 | −0.48 | |
| GVIHMLMQA | 240 | No | No | ~~~HMLMQ~ | 291 | 0.30 | −1.10 | −0.22 | −0.39 | 0.55 | 0.70 | 0.23 | 0.26 | 0.15 | |
| VIHMLMQAY | 241 | No | No | ~~~MLMQA~ | 292 | −0.31 | −0.05 | 0.28 | −2.61 | −1.67 | −0.18 | −0.08 | 0.36 | −1.75 | |
| IHMLMQAYK | 242 | No | No | ~~~LMQAY~ | 293 | 0.64 | 1.16 | 0.83 | 0.64 | −0.70 | −0.18 | 0.73 | −1.38 | 0.78 | |
| RIPDRPYSL | 243 | No | No | ~~~DRPYS~ | 294 | −1.62 | −2.94 | −4.59 | −0.86 | −0.31 | 0.06 | −0.22 | −0.19 | −0.54 | |
| IPDRPYSLF | 244 | No | Yes | ~~~RPYSL~ | 295 | −3.00 | −2.09 | −3.18 | −0.76 | −1.90 | −1.49 | −0.18 | 0.22 | −2.25 | |
| PDRPYSLFE | 245 | No | No | ~~~PYSLF~ | 296 | 0.75 | 0.74 | −0.04 | −0.23 | 0.61 | −0.25 | −0.30 | −1.55 | 1.08 | |
| DRPYSLFEG | 246 | No | No | ~~~YSLFE~ | 297 | 1.42 | −0.15 | −0.31 | 1.11 | 0.30 | −0.63 | −2.34 | −1.12 | 2.14 | |
| RPYSLFEGM | 247 | No | No | ~~~SLFEG~ | 298 | −3.11 | −1.20 | −0.11 | −1.33 | −0.88 | −1.63 | 0.19 | −0.68 | −2.24 | |
| PYSLFEGMA | 248 | No | Yes | ~~~LFEGM~ | 299 | 0.90 | −0.53 | −0.66 | 0.22 | −0.22 | 0.20 | 0.46 | 1.06 | 0.77 | |
| YSLFEGMAG | 249 | No | No | ~~~FEGMA~ | 300 | −0.78 | −0.98 | 0.03 | −0.50 | 0.30 | 0.46 | 0.02 | −0.21 | −1.44 | |
| SLFEGMAGA | 250 | No | No | ~~~EGMAG~ | 301 | −0.81 | −0.81 | 0.56 | −0.96 | −1.23 | −0.17 | −0.32 | 0.67 | −0.43 | |
| LFEGMAGAI | 251 | No | No | ~~~GMAGA~ | 302 | −0.59 | −1.97 | 1.47 | −1.03 | −1.64 | −1.56 | −0.02 | 1.01 | −1.02 | |
| FEGMAGAIH | 252 | No | No | ~~~MAGAI~ | 303 | 1.06 | 1.69 | 0.34 | −0.62 | −0.53 | −1.16 | −1.68 | −0.24 | −0.89 | |
| EGMAGAIHF | 253 | No | Yes | ~~~AGAIH~ | 304 | 0.19 | 0.31 | 1.20 | −2.53 | −0.63 | −1.35 | −0.12 | 0.91 | −2.09 | |
| GMAGAIHFL | 254 | No | Yes | ~~~GAIHF~ | 305 | 1.20 | 0.68 | 3.37 | −1.24 | −0.11 | −0.96 | −0.32 | 0.35 | 0.48 | |
| MAGAIHFLS | 255 | No | No | ~~~AIHFL~ | 306 | −0.76 | 0.19 | 0.40 | 0.51 | 0.40 | 0.62 | 0.22 | 0.65 | −1.80 | |
| AGAIHFLSD | 256 | No | No | ~~~IHFLS~ | 307 | 0.25 | −0.18 | −0.15 | 1.41 | 1.80 | 1.92 | 0.03 | 0.59 | −0.02 | |

#Footnotes as for Table 1

TABLE 8
Predicted binding of peptides of LANC2 to representative DRB alleles

| | SEQ | | | | SEQ | | | | | | | | | | |
|---|---|---|---|---|---|---|---|---|---|---|---|---|---|---|---|
| | ID | CAT | CAT | TCEM | ID | DRB1 | DRB1 | :DRB1 | DRB1 | DRB1 | DRB1 | DRB1 | DRB1 | DRB3 | DRB3 |
| peptide | NO: | S# | L | IIa | NO: | 0701 | 0801 | 0901 | 1101 | 1201 | 1301 | 1501 | 1602 | 0101 | 0202 |
| SAYTGWTGIALLYLQ | 308 | No | No | GW~G~AL | 371 | −1.79 | −0.26 | −1.25 | −0.58 | −0.67 | −0.35 | −0.96 | −0.10 | −1.19 | 0.05 |
| AYTGWTGIALLYLQL | 309 | No | No | WT~1~LL | 372 | −1.39 | −1.17 | −2.10 | 0.20 | −1.30 | −0.73 | −1.58 | −0.79 | −1.57 | −2.06 |
| YTGWTGIALLYLQLY | 310 | Yes | Yes | TG~A~LY | 373 | −1.14 | −1.86 | −1.27 | −0.55 | −2.22 | −1.14 | −1.53 | 0.16 | −1.36 | −1.14 |
| TGWTGIALLYLQLYR | 311 | Yes | Yes | GI~L~YL | 374 | −1.08 | −1.31 | −0.73 | −0.83 | −2.39 | −1.63 | −1.50 | 0.57 | −0.95 | −0.81 |
| GWTGIALLYLQLYRV | 312 | Yes | Yes | IA~L~LQ | 375 | −1.83 | −2.22 | −1.51 | −1.00 | −1.88 | −1.63 | −1.93 | 0.05 | −0.81 | −1.30 |
| WTGIALLYLQLYRVT | 313 | Yes | No | AL~Y~QL | 376 | −2.05 | −2.53 | −1.70 | −1.81 | −2.22 | −2.48 | −1.85 | 0.15 | −1.39 | −1.37 |
| TGIALLYLQLYRVTC | 314 | No | Yes | LL~L~LY | 377 | −1.28 | −1.63 | −0.99 | −1.73 | −2.58 | −1.28 | −1.76 | −1.30 | −1.10 | −2.46 |
| GIALLYLQLYRVTCD | 315 | Yes | :Yes | LY~Q~YR | 378 | −0.80 | −1.43 | −1.40 | −1.20 | −2.44 | −1.22 | −0.94 | 0.31 | −1.11 | −1.02 |
| IALLYLQLYRVTCDQ | 316 | No | Yes | YL~L~RV | 379 | −1.61 | −1.83 | −0.90 | −1.81 | −0.72 | −2.90 | −1.32 | −0.45 | −1.95 | −1.83 |
| ALLYLQLYRVTCDQT | 317 | No | Yes | LQ~Y~VT | 380 | 0.01 | −2.11 | −1.25 | −2.46 | −1.66 | −1.20 | −0.95 | −0.62 | 0.40 | −0.96 |
| LLYLQLYRVTCDQTY | 318 | No | Yes | QL~R~TC | 381 | −1.53 | −1.49 | −1.23 | −1.64 | −0.59 | −1.54 | −0.93 | 0.97 | −1.31 | −1.27 |
| LYLQLYRVTCDQTYL | 319 | No | Yes | LY~V~CD | 382 | −1.68 | −1.05 | −0.41 | −0.81 | −0.88 | −0.96 | −1.35 | −0.35 | −0.85 | −1.20 |
| YLQLYRVTCDQTYLL | 320 | No | No | YR~T~DQ | 383 | −1.35 | −1.32 | −1.26 | −0.93 | −0.41 | −0.88 | −0.76 | −0.67 | −2.36 | −1.24 |
| LQLYRVTCDQTYLLR | 321 | No | No | RV~C~QT | 384 | −1.45 | −0.74 | −0.49 | −0.46 | −0.22 | −0.89 | −0.70 | −1.62 | −2.67 | −1.87 |
| LPDELLYGRAGYLYA | 322 | Yes | Yes | LL~G~AG | 385 | −1.95 | −0.86 | −0.76 | −0.78 | −0.34 | −0.97 | −1.93 | 0.67 | −1.79 | 0.41 |
| PDELLYGRAGYLYAL | 323 | Yes | Yes | LY~R~GY | 386 | −1.92 | −0.87 | −1.26 | −1.10 | −1.71 | −0.50 | −2.03 | −0.97 | −1.44 | −1.31 |
| DELLYGRAGYLYALL | 324 | Yes | Yes | YG~A~YL | 387 | −1.78 | −1.22 | −1.31 | −0.65 | −1.57 | −1.00 | −1.43 | −2.66 | −1.63 | −2.29 |
| ELLYGRAGYLYALLY | 325 | Yes | No | GR~G~LY | 388 | −2.13 | −1.01 | −1.10 | −0.70 | −2.31 | −1.20 | −1.47 | 0.30 | −1.62 | −1.28 |
| LLYGRAGYLYALLYL | 326 | Yes | No | RA~Y~YA | 389 | −1.82 | −1.51 | −1.41 | −0.57 | −1.36 | −1.79 | −2.17 | 0.85 | −1.20 | −1.23 |
| LYGRAGYLYALLYLN | 327 | No | No | AG~L~AL | 390 | −1.59 | −1.27 | −1.60 | −0.81 | −1.70 | −1.32 | −1.98 | −0.68 | −0.68 | −0.69 |
| YGRAGYLYALLYLNT | 328 | No | No | GY~Y~LL | 391 | −0.91 | −1.27 | −1.54 | −0.66 | −1.22 | −1.14 | −1.81 | 1.34 | −0.11 | −0.87 |
| GRAGYLYALLYLNTE | 329 | No | No | YL~A~LY | 392 | −0.76 | −0.86 | −1.21 | −0.88 | −1.20 | −0.91 | −1.43 | −0.61 | 0.08 | −0.74 |
| RAGYLYALLYLNTEI | 330 | Yes | No | LY~L~YL | 393 | −1.30 | −2.07 | −1.35 | −1.12 | −1.69 | −2.17 | −2.13 | 0.10 | 0.10 | −2.02 |
| AGYLYALLYLNTEIG | 331 | Yes | No | YA~L~LN | 394 | −1.07 | −2.14 | −2.13 | −1.35 | −2.73 | −0.89 | −1.01 | −0.75 | −0.34 | −1.20 |
| GYLYALLYLNTEIGP | 332 | No | Yes | AL~Y~NT | 395 | −1.18 | −2.41 | −1.02 | −0.90 | −1.70 | −1.63 | −1.67 | −0.67 | −0.95 | −1.36 |
| YLYALLYLNTEIGPG | 333 | Yes | No | LL~L~TE | 396 | −1.67 | −2.55 | −1.36 | −2.06 | −0.89 | −1.23 | −2.15 | −0.25 | −0.24 | −1.75 |

| | | | | | | | | | | | | | | | |
|---|---|---|---|---|---|---|---|---|---|---|---|---|---|---|---|
| LYALLYLNTEIGPGT | 334 | No | No | LY~N~EI | 397 | −1.14 | −2.17 | −1.44 | −1.37 | −0.63 | −1.41 | −1.21 | −1.07 | 0.62 | −1.53 |
| YALLYLNTEIGPGTV | 335 | No | No | YL~T~IG | 398 | −1.64 | −1.24 | −1.57 | −1.58 | −1.27 | −0.52 | −1.32 | −0.76 | −1.78 | −2.14 |

| | | | | | | | | | | | | | | | |
|---|---|---|---|---|---|---|---|---|---|---|---|---|---|---|---|
| ALLYLNTEIGPGTVC | 336 | Yes | Yes | LN~E~GP | 399 | −1.14 | −0.50 | −2.72 | −0.66 | −0.42 | 0.03 | −0.72 | −3.41 | −1.40 | −1.13 |
| LLYLNTEIGPGTVCE | 337 | Yes | Yes | NT~I~PG | 400 | −0.92 | −0.33 | −1.34 | −0.20 | −0.53 | 0.40 | 0.25 | −1.08 | 0.02 | −1.69 |
| AHGMAGIYYMLMQPA | 338 | No | No | AG~Y~ML | 401 | −0.03 | −1.08 | −1.28 | −0.28 | −1.67 | −1.09 | −1.32 | 0.70 | −0.31 | 0.21 |
| HGMAGIYYMLMQPAA | 339 | No | Yes | GI~Y~LM | 402 | −0.53 | −1.35 | −0.38 | −0.67 | −1.05 | −0.59 | −0.46 | 1.27 | −0.96 | 0.04 |
| GMAGIYYMLMQPAAK | 340 | No | Yes | IY~M~MQ | 403 | −0.24 | −0.64 | 0.09 | −0.93 | −0.65 | −0.31 | −1.20 | 0.54 | 0.80 | 0.16 |
| MAGIYYMLMQPAAKV | 341 | No | Yes | YY~L~QP | 404 | −1.32 | −2.49 | −1.55 | −1.60 | −0.94 | −2.54 | −2.16 | −0.01 | −1.29 | −1.07 |
| AGIYYMLMQPAAKVD | 342 | No | Yes | YM~M~PA | 405 | −1.11 | −2.15 | −2.34 | −1.85 | −0.99 | −0.75 | −2.18 | −1.17 | −0.54 | −0.86 |
| GIYYMLMQPAAKVDQ | 343 | No | No | ML~Q~AA | 406 | −1.35 | −1.55 | −2.15 | −1.76 | −1.55 | −1.07 | −1.55 | −0.59 | −0.33 | −2.56 |
| IYYMLMQPAAKVDQE | 344 | No | No | LM~P~AK | 407 | −1.83 | −1.92 | −1.91 | −1.36 | −1.41 | −1.43 | −1.23 | 0.07 | −0.93 | −2.55 |
| YYMLMQPAAKVDQET | 345 | No | Yes | MQ~A~KV | 408 | −0.69 | −1.19 | −1.20 | −1.34 | −0.43 | −1.26 | −0.34 | −0.61 | 0.17 | −2.13 |
| RLVHWCHGAPGVIHM | 346 | No | Yes | WC~G~PG | 409 | −2.73 | −0.62 | −1.29 | −0.45 | −0.66 | 0.17 | −1.53 | −0.77 | −1.47 | −1.81 |
| LVHWCHGAPGVIHML | 347 | No | Yes | CH~A~GV | 410 | −1.35 | −0.27 | −2.05 | −0.27 | −0.51 | −0.11 | −0.62 | 0.21 | −1.80 | −1.34 |
| VHWCHGAPGVIHMLM | 348 | No | Yes | HG~P~VI | 411 | −1.41 | −0.21 | −1.28 | 0.58 | −1.09 | −0.37 | −0.82 | −0.10 | −1.74 | −1.87 |
| HWCHGAPGVIHMLMQ | 349 | Yes | Yes | GA~G~IH | 412 | −0.89 | 0.70 | −0.56 | 0.84 | −0.86 | 0.61 | −0.52 | −0.39 | −0.44 | 0.23 |
| WCHGAPGVIHMLMQA | 350 | Yes | No | AP~V~HM | 413 | −0.72 | 0.38 | −0.21 | 1.31 | −0.14 | 0.33 | −0.44 | −0.20 | 1.33 | −0.08 |
| CHGAPGVIHMLMQAY | 351 | Yes | No | PG~I~ML | 414 | 0.34 | −0.11 | 0.16 | 1.07 | −2.14 | −0.08 | −0.01 | −0.18 | 0.03 | 1.03 |
| HGAPGVIHMLMQAYK | 352 | Yes | Yes | GV~H~LM | 415 | −0.03 | −0.19 | 1.36 | 0.61 | −0.37 | 0.05 | 0.39 | 1.14 | −0.31 | 0.14 |
| GAPGVIHMLMQAYKV | 353 | Yes | Yes | VI~M~MQ | 416 | −0.12 | −0.65 | −0.15 | 0.08 | −1.42 | −1.04 | −0.94 | 0.45 | 0.40 | 0.28 |
| APGVIHMLMQAYKVF | 354 | Yes | Yes | IH~L~QA | 417 | −1.07 | −1.97 | −0.50 | −1.06 | −2.39 | −1.28 | −1.17 | 1.98 | −2.04 | −0.02 |
| PGVIHMLMQAYKVFK | 355 | Yes | Yes | HM~M~AY | 418 | −1.55 | −1.63 | −1.52 | −1.74 | −2.21 | −1.01 | −3.27 | −0.57 | −1.10 | −1.52 |
| GVIHMLMQAYKVFKE | 356 | No | No | ML~Q~YK | 419 | −1.60 | −1.19 | −1.62 | −1.57 | −2.43 | −1.85 | −2.88 | −1.56 | −1.26 | −1.84 |
| VIHMLMQAYKVFKEE | 357 | No | No | LM~A~KV | 420 | −0.82 | −1.16 | −0.59 | −0.89 | −1.45 | −1.43 | −2.00 | 0.62 | −0.91 | −1.48 |
| IHMLMQAYKVFKEEK | 358 | No | No | MQ~Y~VF | 421 | −0.21 | −1.06 | −0.33 | −1.19 | −1.17 | −1.15 | −0.95 | −0.04 | −0.56 | −1.68 |
| PDRPYSLFEGMAGAI | 359 | No | No | YS~F~GM | 422 | −1.05 | −0.82 | −1.76 | −0.36 | 0.27 | 0.31 | −0.15 | −2.04 | −0.13 | −0.55 |
| DRPYSLFEGMAGAIH | 360 | Yes | Yes | SL~E~MA | 423 | −0.95 | −0.33 | −1.43 | 0.08 | −0.75 | 0.46 | −1.00 | −1.50 | 0.28 | −1.13 |
| RPYSLFEGMAGAIHF | 361 | Yes | Yes | LF~G~AG | 424 | −2.63 | −0.66 | −1.98 | −0.55 | −0.89 | 0.17 | −0.91 | −0.10 | −1.41 | −1.15 |
| PYSLFEGMAGAIHFL | 362 | Yes | Yes | FE~M~GA | 425 | −3.41 | −0.79 | −2.92 | −0.09 | −1.39 | −0.38 | −1.27 | −1.21 | −0.90 | −1.36 |
| YSLFEGMAGAIHFLS | 363 | Yes | Yes | EG~A~AI | 426 | −2.37 | −0.51 | −1.38 | −0.13 | −1.98 | 0.13 | −0.73 | −0.85 | −2.27 | −2.01 |
| SLFEGMAGAIHFLSD | 364 | Yes | Yes | GM~G~IH | 427 | −1.25 | 0.43 | −0.59 | 0.65 | 0.01 | 0.06 | −0.54 | 1.23 | −0.23 | −0.39 |
| LFEGMAGAIHFLSDV | 365 | Yes | Yes | MA~A~HF | 428 | −0.86 | −0.09 | −0.60 | 0.42 | −0.70 | −0.31 | −0.26 | −0.21 | 0.20 | −0.05 |
| FEGMAGAIHFLSDVL | 366 | Yes | No | AG~I~FL | 429 | −0.76 | −0.29 | −0.48 | 0.68 | −1.02 | −0.22 | 0.14 | −0.23 | 0.46 | 0.73 |
| EGMAGAIHFLSDVLG | 367 | Yes | Yes | GA~H~LS | 430 | −0.69 | 0.34 | −0.17 | 0.40 | −0.52 | 0.80 | −0.74 | −0.37 | 0.04 | 0.45 |
| GMAGAIHFLSDVLGP | 368 | No | Yes | AI~F~SD | 431 | −0.30 | −0.26 | −0.28 | 0.15 | −1.34 | 0.14 | −0.61 | 0.93 | 0.58 | 0.45 |
| MAGAIHFLSDVLGPE | 369 | No | Yes | IH~L~DV | 432 | −0.34 | −0.94 | −1.11 | −0.50 | −1.31 | −0.71 | −1.29 | 0.72 | −0.52 | −0.20 |
| AGAIHFLSDVLGPET | 370 | No | Yes | HF~S~VL | 433 | −0.29 | −0.63 | −0.98 | −0.58 | −1.20 | 0.05 | −0.87 | 0.31 | −1.16 | −0.53 |

#Footnotes as for Table 1

TABLE 9
Peptides designed to optimize binding for specific MHC I alleles
of T cell exposed motifs found in LANC2

| | | | | | | | | |
|---|---|---|---|---|---|---|---|---|
| | | | Predicted | | | Predicted | | |
| | | SEQ | binding of | | SEQ | binding of | | SEQ |
| | originating | ID | originating | proposed | ID | proposed | TCEM | ID |
| pos. | peptide | NO: | peptide | peptide | NO: | peptide | core | NO.: |

Peptides Proposed for A0801

| | | | | | | | | |
|---|---|---|---|---|---|---|---|---|
| 196 | LLYGRAGYL | 221 | −1.52 | IYWGRAGYV | 434 | −2.03 | GRAGY | 454 |
| 196 | LLYGRAGYL | 221 | −1.52 | HLYGRAGYV | 435 | −1.93 | GRAGY | 454 |
| 196 | LLYGRAGYL | 221 | −1.52 | MGWGRAGYL | 436 | −1.88 | GRAGY | 454 |
| 196 | LLYGRAGYL | 221 | −1.52 | YPWGRAGYL | 437 | −1.82 | GRAGY | 454 |

Peptides proposed for nA_0217

| | | | | | | | | |
|---|---|---|---|---|---|---|---|---|
| 196 | LLYGRAGYL | 221 | −0.44 | LGDGRAGYW | 438 | −2.05 | GRAGY | 454 |
| 196 | LLYGRAGYL | 221 | −0.44 | KGRGRAGYW | 439 | −2.04 | GRAGY | 454 |
| 196 | LLYGRAGYL | 221 | −0.44 | WVKGRAGYR | 440 | −2.02 | GRAGY | 454 |
| 196 | LLYGRAGYL | 221 | −0.44 | RVKGRAGYL | 441 | −2.02 | GRAGY | 454 |

Peptides proposed forA_3101

| | | | | | | | | |
|---|---|---|---|---|---|---|---|---|
| 416 | SLFEGMAGA | 250 | −0.07 | FAAEGMAGR | 442 | −2.05 | EGMAG | 455 |
| 416 | SLFEGMAGA | 250 | −0.07 | PVAEGMAGR | 443 | −2.03 | EGMAG | 455 |
| 416 | SLFEGMAGA | 250 | −0.07 | GIVEGMAGR | 444 | −2.02 | EGMAG | 455 |
| 416 | SLFEGMAGA | 250 | −0.07 | FVGEGMAGR | 445 | −2.01 | EGMAG | 455 |

Peptides proposed for A3301

| | | | | | | | | |
|---|---|---|---|---|---|---|---|---|
| 416 | SLFEGMAGA | 250 | −0.02 | HMLEGMAGK | 446 | −2.07 | EGMAG | 455 |
| 416 | SLFEGMAGA | 250 | −0.02 | LFGEGMAGR | 447 | −2.05 | EGMAG | 455 |
| 416 | SLFEGMAGA | 250 | −0.02 | ALIEGMAGR | 448 | −2.05 | EGMAG | 455 |
| 416 | SLFEGMAGA | 250 | −0.02 | SFLEGMAGR | 449 | −2.03 | EGMAG | 455 |
| 415 | YSLFEGMAG | 249 | 0.18 | QEAFEGMAR | 450 | −2.08 | FEGMA | 456 |
| 415 | YSLFEGMAG | 249 | 0.18 | YDQFEGMAR | 451 | −2.05 | FEGMA | 456 |
| 415 | YSLFEGMAG | 249 | 0.18 | EYSFEGMAK | 452 | −2.05 | FEGMA | 456 |
| 415 | YSLFEGMAG | 249 | 0.18 | YQEFEGMAR | 453 | −2.05 | FEGMA | 456 |

TABLE 10
Predicted binding of peptides of SEPT14 to representative MHC I A and MHC IB alleles

| | SEQ | | | | SEQ | | | | | | | | | | |
|---|---|---|---|---|---|---|---|---|---|---|---|---|---|---|---|
| selected | ID | | | | ID | | | | | | | | | | |
| peptide | NO.: | CatS | CatL | TCEM I | NO.: | A0101 | A0201 | A0202 | A0203 | A0206 | A0211 | A0212 | A0301 | A0801 | A1101 |
| IRQGFTFNI | 457 | No | No | ~~~GFTFN~ | 488 | −0.12 | −0.51 | −2.29 | −0.94 | −1.88 | 0.30 | 0.13 | −0.06 | −1.04 | 0.31 |
| RQGFTFNIL | 458 | No | No | ~~~FTFNI~ | 489 | −0.17 | −1.14 | −1.02 | −0.70 | −1.14 | 0.87 | 0.86 | 0.66 | −0.51 | 0.97 |
| QGFTFNILC | 459 | Yes | Yes | ~~~TFNIL~ | 490 | −0.54 | −1.65 | −0.29 | −0.98 | −1.52 | −0.84 | −0.72 | −0.18 | −1.12 | −0.63 |
| GFTFNILCV | 460 | No | No | ~~~FNILC~ | 491 | 4.48 | −1.91 | −1.83 | −2.16 | −1.68 | −2.03 | −2.55 | 0.05 | −1.55 | 0.41 |
| FTFNILCVG | 461 | No | No | ~~~NILCV~ | 492 | −1.24 | −3.31 | −1.82 | −2.04 | −2.89 | −2.29 | −2.26 | 0.18 | −1.19 | −0.34 |
| TFNILCVGE | 462 | No | No | ~~~ILCVG~ | 493 | 0.07 | 0.49 | −0.25 | −0.32 | −0.50 | −0.98 | −0.73 | 0.39 | −0.01 | −0.01 |
| FNILCVGET | 463 | No | No | ~~~LCVGE~ | 494 | −0.64 | −0.99 | −0.66 | −0.34 | −1.10 | 0.08 | −0.07 | 0.98 | 0.09 | 0.35 |
| NILCVGETG | 464 | No | Yes | ~~~CVGET~ | 495 | 0.54 | −1.63 | −1.73 | −2.09 | −1.57 | −2.17 | −2.64 | 0.32 | −0.34 | 0.73 |
| ILCVGETGI | 465 | No | No | ~~~VGETG~ | 496 | 0.01 | −2.15 | −1.97 | −1.51 | −1.32 | −1.67 | −1.85 | 0.08 | −1.20 | 1.45 |
| LCVGETGIG | 466 | No | No | ~~~GETGI~ | 497 | −0.73 | 0.42 | −1.44 | −1.74 | −1.23 | −2.12 | −2.37 | 0.50 | 0.67 | 0.91 |
| RVHVCLYFI | 467 | No | No | ~~~VCLYF~ | 498 | −1.22 | −3.26 | −1.55 | −1.51 | −1.45 | −1.84 | −1.78 | −0.90 | −0.69 | −0.34 |
| VHVCLYFIS | 468 | No | No | ~~~CLYFI~ | 499 | −0.83 | −0.97 | −1.20 | −0.87 | −1.82 | −0.89 | −1.12 | 0.17 | −0.02 | −0.52 |
| HVCLYFISP | 469 | No | No | ~~~LYFIS~ | 500 | −0.94 | −1.16 | −0.37 | −0.60 | −0.82 | −0.68 | 1.12 | −2.28 | −0.13 | −2.21 |
| VCLYFISPT | 470 | No | Yes | ~~~YFISP~ | 501 | 0.10 | −0.87 | −0.17 | −0.86 | −0.97 | −1.36 | −1.01 | −1.26 | −0.30 | −0.16 |
| CLYFISPTG | 471 | No | No | ~~~FISPT~ | 502 | −0.07 | −2.85 | −0.90 | −1.62 | −0.65 | −1.71 | −1.34 | −0.37 | −1.84 | 0.41 |
| LYFISPTGH | 472 | No | No | ~~~ISPTG~ | 503 | 0.50 | 0.68 | −0.06 | −0.54 | −0.77 | −0.05 | −0.49 | −0.33 | 0.79 | −0.06 |
| YFISPTGHS | 473 | No | Yes | ~~~SPTGH~ | 504 | 0.92 | −0.85 | −1.70 | −2.19 | −1.40 | −1.96 | −2.19 | −0.01 | −0.70 | 0.50 |
| FISPTGHSL | 474 | No | No | ~~~PTGHS~ | 505 | −0.50 | −2.27 | −2.35 | −2.10 | −1.49 | −2.01 | −2.17 | 0.33 | −1.38 | 0.69 |
| NLDSKVNII | 475 | No | No | ~~~SKVNI~ | 506 | −0.67 | −1.70 | −1.71 | −1.56 | −0.24 | −1.36 | −1.57 | 0.22 | −0.57 | 0.73 |
| LDSKVNIIP | 476 | No | Yes | ~~~KVNII~ | 507 | −2.39 | 0.51 | −0.16 | 1.53 | −0.16 | 0.44 | 1.21 | −0.49 | 0.22 | −0.87 |
| DSKVNIIPL | 477 | No | No | ~~~VNIIP~ | 508 | −1.11 | −0.01 | −0.32 | −0.55 | −0.27 | −0.29 | 0.33 | 0.31 | −1.30 | 0.30 |
| SKVNIIPLI | 478 | No | No | ~~~NIIPL~ | 509 | −0.09 | −1.12 | −1.70 | −1.36 | −1.96 | 0.26 | −0.07 | 0.20 | −0.28 | 0.06 |
| KVNIIPLIA | 479 | No | No | ~~~IIPLI~ | 510 | −1.59 | −2.47 | −0.68 | −1.53 | −1.42 | −1.93 | −2.02 | −0.63 | −0.40 | −0.57 |
| VNIIPLIAK | 480 | No | No | ~~~IPLIA~ | 511 | −0.65 | 0.00 | 0.45 | 0.39 | −0.22 | 1.34 | 0.77 | −2.44 | 0.57 | −2.85 |
| NIIPLIAKA | 481 | No | No | ~~~PLIAK~ | 512 | 0.51 | −2.32 | −2.15 | −2.63 | −2.72 | −2.19 | 2.48 | −0.01 | −0.96 | −0.30 |
| IIPLIAKAD | 482 | No | No | ~~~LIAKA~ | 513 | 0.30 | 0.67 | 0.43 | −0.08 | −0.09 | −0.77 | −0.09 | −0.76 | 0.36 | −0.04 |
| IPLIAKADT | 483 | No | No | ~~~IAKAD~ | 514 | 0.37 | 0.87 | −0.08 | −0.29 | −0.05 | 0.24 | 0.13 | 0.54 | −0.17 | 0.58 |
| PLIAKADTI | 484 | No | No | ~~~AKADT~ | 515 | 1.76 | −1.89 | −2.26 | −2.50 | −1.77 | −1.90 | −2.48 | 0.61 | −0.99 | 0.96 |
| LIAKADTIS | 485 | No | Yes | ~~~KADTI~ | 516 | 0.31 | 0.78 | −0.98 | −1.27 | −0.21 | −1.52 | −1.83 | −0.12 | 0.58 | 0.44 |
| 9-mer | | CatS | CatL | TCEMI | | B0702 | B0801 | B0802 | B0803 | B1501 | B1502 | B1503 | B2703 | B2705 | B3501 |
| IRQGFTFNI | 459 | No | No | ~~~GFTFN~ | 517 | −0.10 | −0.68 | −0.56 | −0.63 | 0.31 | −1.32 | −1.63 | −0.92 | −2.65 | 0.42 |
| RQGFTFNIL | 460 | No | No | ~~~FTFNI~ | 518 | −1.51 | −1.44 | −0.34 | 0.07 | −0.85 | −0.35 | −0.75 | −1.12 | −0.69 | −0.54 |
| QGFTFNILC | 461 | Yes | Yes | ~~~TFNIL~ | 519 | −0.79 | −0.95 | 0.03 | −0.29 | 0.51 | −2.09 | −0.34 | −2.12 | −0.26 | 1.32 |
| GFTFNILCV | 462 | No | No | ~~~FNILC~ | 520 | 0.54 | −1.73 | 1.07 | −0.35 | 0.52 | 0.57 | 0.29 | 0.16 | 0.75 | 0.90 |
| FTFNILCVG | 463 | No | No | ~~~NILCV~ | 521 | −0.24 | −1.13 | 1.06 | −0.44 | −1.15 | −0.94 | −0.02 | −0.28 | −0.36 | −1.30 |
| TFNILCVGE | 464 | No | No | ~~~ILCVG~ | 522 | 0.93 | 0.39 | 0.88 | −1.20 | 0.15 | −0.09 | −0.06 | 0.58 | 0.60 | 1.10 |
| FNILCVGET | 465 | No | No | ~~~LCVGE~ | 523 | 1.23 | 0.37 | 0.81 | 0.62 | −1.20 | 1.00 | −0.12 | −0.07 | −0.48 | −0.73 |
| NILCVGETG | 466 | No | Yes | ~~~CVGET~ | 524 | 0.36 | 1.14 | 0.35 | −0.23 | −0.25 | −0.97 | −0.53 | −0.34 | 0.13 | −0.33 |
| ILCVGETGI | 467 | No | No | ~~~VGETG~ | 525 | −2.04 | 0.00 | 0.79 | 0.79 | −1.29 | −2.68 | −0.31 | −0.09 | 2.41 | 2.16 |
| LCVGETGIG | 468 | No | No | ~~~GETGI~ | 526 | −0.37 | −0.10 | 1.59 | −1.56 | −0.52 | −1.12 | −0.16 | −0.11 | 1.36 | 0.02 |
| RVHVCLYFI | 469 | No | No | ~~~VCLYF~ | 527 | −1.62 | −1.36 | 0.51 | −0.31 | −0.94 | 0.17 | −0.04 | −0.26 | −0.82 | −1.48 |
| VHVCLYFIS | 470 | No | No | ~~~CLYFI~ | 528 | 0.90 | 0.26 | 1.14 | 0.12 | 0.36 | −1.49 | −0.78 | 0.18 | −1.68 | −0.04 |
| HVCLYFISP | 471 | No | No | ~~~LYFIS~ | 529 | −0.15 | −0.95 | 0.56 | 1.47 | −0.30 | −1.13 | 0.99 | −0.19 | 0.39 | −1.00 |
| VCLYFISPT | 472 | No | Yes | ~~~YFISP~ | 530 | −0.24 | −0.38 | 0.71 | −1.03 | −0.30 | −1.78 | −0.08 | −0.51 | −0.05 | 0.58 |
| CLYFISPTG | 473 | No | No | ~~~FISPT~ | 531 | −0.17 | −1.75 | − 0.16 | −0.19 | 0.21 | −0.91 | 0.15 | −0.58 | −0.01 | −1.06 |
| LYFISPTGH | 474 | No | No | ~~~ISPTG~ | 532 | 0.32 | −0.24 | −0.20 | −0.13 | −0.92 | −2.15 | −1.29 | −0.43 | −0.76 | −1.30 |
| YFISPTGHS | 475 | No | Yes | ~~~SPTGH~ | 533 | 0.88 | −0.20 | 0.55 | −0.30 | −0.28 | −1.34 | −0.12 | −0.16 | −0.35 | 0.48 |
| FISPTGHSL | 476 | No | No | ~~~PTGHS~ | 534 | −1.86 | −1.26 | −0.65 | −1.15 | −1.36 | −1.86 | −0.68 | −0.27 | 1.23 | −1.74 |
| NLDSKVNII | 477 | No | No | ~~~SKVNI~ | 535 | 0.10 | −2.08 | −0.26 | −0.98 | −1.06 | −1.15 | 0.04 | −0.23 | 1.17 | −0.15 |
| LDSKVNIIP | 478 | No | Yes | ~~~KVNI~ | 536 | −0.12 | 0.09 | −0.35 | −0.29 | −0.47 | 1.80 | −0.39 | −0.14 | 0.24 | −0.45 |
| DSKVNIIPL | 479 | No | No | ~~~VNIIP~ | 537 | −1.01 | −2.22 | −0.66 | −2.06 | −0.75 | 0.33 | 0.29 | −0.01 | 0.06 | −0.62 |
| SKVNIIPLI | 480 | No | No | ~~~NIIPL~ | 538 | −0.72 | −0.49 | 0.45 | 1.00 | −0.08 | 0.06 | −1.00 | −0.12 | −2.11 | −0.60 |
| KVNIIPLIA | 481 | No | No | ~~~IIPLI~ | 539 | −0.90 | −0.40 | 0.26 | −1.75 | −0.56 | 0.04 | 0.21 | −0.43 | −0.08 | −0.51 |
| VNIIPLIAK | 482 | No | No | ~~~IPLIA~ | 540 | −0.04 | 0.81 | 1.70 | 0.78 | −0.41 | −0.65 | 0.30 | 0.22 | −0.59 | −0.27 |
| NIIPLIAKA | 483 | No | No | ~~~PLIAK~ | 541 | 0.09 | −0.64 | 0.92 | −0.34 | −0.62 | −1.66 | 0.63 | −0.23 | 0.00 | 0.24 |
| IIPLIAKAD | 484 | No | No | ~~~LIAKA~ | 542 | 0.67 | 1.12 | 0.41 | −1.16 | 0.95 | −0.29 | 0.76 | −0.06 | 0.33 | 0.20 |
| IPLIAKADT | 485 | No | No | ~~~IAKAD~ | 543 | −2.17 | 0.96 | 0.02 | −0.03 | 1.56 | −0.90 | 0.77 | −0.15 | 1.04 | 1.17 |
| PLIAKADTI | 486 | No | No | ~~~AKADT~ | 544 | −0.59 | −1.17 | 0.39 | −0.77 | 1.32 | −3.54 | −0.45 | −0.06 | 0.12 | −0.85 |
| LIAKADTIS | 487 | No | Yes | ~~~KADTI~ | 545 | −1.23 | −0.02 | −0.35 | −0.49 | −0.24 | −0.11 | 0.81 | −0.52 | 1.14 | −0.34 |

#Footnotes as for Table 1

TABLE 11
Predicted binding of peptides of SEPT14 to representative DRB alleles

| | SEQ | | SEQ | | | | | | | | | | | | |
|---|---|---|---|---|---|---|---|---|---|---|---|---|---|---|---|
| | ID | TCEM | ID | DRB1 | DRB1 | DRB1 | DRB1 | DRB1 | DRB1 | DRB1 | DRB1 | DRB1 | DRB1 | DRB3 | DRB3 |
| peptide | NO.: | IIa | NO.: | 0101 | 0301 | 0401 | 0701 | 0801 | 0901 | 1101 | 1201 | 1301 | 1501 | 0101 | 0202 |
| QLVSRSIRQGFTFNI | 546 | RS~R~GF | 575 | −0.28 | −1.00 | −0.27 | −2.38 | −0.34 | −0.42 | −0.02 | −0.64 | −0.80 | −0.78 | −1.10 | −0.12 |
| LVSRSIRQGFTFNIL | 547 | SI~Q~FT | 576 | −0.75 | −1.04 | −0.68 | −1.22 | −0.34 | −0.71 | −0.53 | −0.65 | −1.53 | −0.88 | −1.43 | 0.19 |
| VSRSIRQGFTFNILC | 548 | IR~G~TF | 577 | −0.40 | −0.32 | 0.39 | −1.94 | −0.03 | −1.45 | 0.43 | −0.90 | 0.29 | −1.20 | −1.81 | −0.84 |
| SRSIRQGFTFNILCV | 549 | RQ~F~FN | 578 | −1.10 | −0.21 | −0.26 | −1.62 | −0.35 | −1.24 | −0.33 | −0.73 | 0.02 | −0.75 | −1.84 | −1.22 |
| RSIRQGFTFNILCVG | 550 | QG~T~NI | 579 | −0.83 | 0.63 | −0.68 | −2.19 | −0.49 | −1.62 | −0.34 | 0.71 | −0.13 | −1.26 | −1.98 | −0.43 |
| SIRQGFTFNILCVGE | 551 | GF~F~IL | 580 | −1.11 | 1.26 | 0.47 | −0.71 | −0.43 | −1.00 | 0.09 | −0.86 | 0.58 | −0.60 | −0.58 | −0.83 |
| IRQGFTFNILCVGET | 552 | FT~N~LC | 581 | −1.03 | 1.51 | −1.08 | −1.07 | −0.42 | −1.32 | −0.23 | −1.48 | 0.56 | −0.62 | −0.26 | −0.98 |
| RQGFTFNILCVGETG | 553 | TF~I~CV | 582 | −1.03 | 0.03 | −1.05 | −1.28 | −1.23 | −0.64 | −0.57 | 0.26 | 0.26 | −0.75 | −0.20 | −1.68 |
| QGFTFNILCVGETGI | 554 | FN~L~VG | 583 | −0.98 | 0.18 | −1.61 | −1.58 | −1.94 | −2.02 | −0.34 | −0.98 | 0.14 | −0.83 | −0.96 | −2.39 |
| GFTFNILCVGETGIG | 555 | NI~C~GE | 584 | −1.52 | 0.59 | −1.20 | −1.83 | −0.47 | −1.05 | −0.15 | −1.29 | 0.89 | −0.57 | 0.08 | −0.84 |
| FTFNILCVGETGIGK | 556 | IL~V~ET | 585 | −1.22 | −0.06 | −1.45 | −0.83 | −0.78 | −1.45 | −0.10 | 0.10 | −0.05 | −0.59 | −0.22 | −1.08 |
| TFNILCVGETGIGKS | 557 | LC~G~TG | 586 | −1.70 | −0.14 | −1.86 | −0.92 | −0.73 | −1.00 | 0.46 | 0.02 | 0.38 | −0.29 | 0.75 | −0.04 |
| HDSRVHVCLYFISPT | 558 | VH~C~YF | 587 | −0.32 | −0.45 | −1.41 | −1.12 | −0.52 | −0.60 | 0.37 | −0.53 | −0.09 | −0.75 | −1.67 | −0.36 |
| DSRVHVCLYFISPTG | 559 | HV~L~FI | 588 | −0.81 | −0.95 | −1.04 | −0.11 | −1.05 | −1.16 | −0.52 | −1.39 | −0.03 | −1.70 | −0.97 | −1.65 |
| SRVHVCLYFISPTGH | 560 | VC~Y~IS | 589 | −0.96 | −0.01 | −1.73 | −1.10 | −1.57 | −1.19 | −1.55 | −1.35 | −1.61 | −1.27 | −0.46 | −0.81 |
| RVHVCLYFISPTGHS | 561 | CL~F~SP | 590 | −1.18 | 0.26 | −1.33 | −1.07 | −2.18 | −1.19 | −1.63 | −2.48 | −0.17 | −1.88 | −0.20 | −1.14 |
| VHVCLYFISPTGHSL | 562 | LY~I~PT | 591 | −1.35 | −0.81 | −1.83 | −1.35 | −2.35 | −2.60 | −3.42 | −2.75 | −1.71 | −1.69 | −0.95 | −1.41 |
| HVCLYFISPTGHSLK | 563 | YF~S~TG | 592 | −2.34 | −0.60 | −2.21 | −1.21 | −1.53 | −0.57 | −2.12 | −1.49 | −0.32 | −1.63 | −0.65 | −1.54 |
| VCLYFISPTGHSLKS | 564 | FI~P~GH | 593 | −2.64 | 0.22 | −1.55 | −1.11 | −1.40 | −2.05 | −1.97 | −1.24 | −2.27 | −1.46 | 0.23 | −2.67 |
| CLYFISPTGHSLKSL | 565 | IS~T~HS | 594 | −1.95 | −0.80 | −2.63 | −1.27 | −2.10 | −1.86 | −1.69 | −1.20 | −1.89 | −1.25 | −0.13 | −1.73 |
| LYFISPTGHSLKSLD | 566 | SP~G~SL | 595 | −2.90 | −0.67 | −1.30 | −1.00 | −0.88 | −1.52 | −0.92 | −1.01 | 1.44 | 0.04 | −0.34 | −1.62 |
| NLDSKVNIIPLIAKA | 567 | KV~I~PL | 596 | −0.79 | 0.88 | 0.51 | −0.73 | −0.88 | −0.77 | −0.51 | −0.52 | −0.22 | −1.53 | 0.34 | 0.26 |
| LDSKVNIIPLIAKAD | 568 | VN~I~LI | 597 | −0.89 | 1.43 | −0.78 | −0.08 | −1.12 | −1.08 | 0.11 | −1.92 | −0.32 | −0.58 | 0.82 | −0.02 |
| DSKVNIIPLIAKADT | 569 | NI~P~IA | 598 | −1.52 | 1.16 | −0.45 | −0.13 | −1.22 | −1.31 | −1.18 | 1.70 | −0.18 | −1.35 | 1.22 | −0.13 |
| SKVNIIPLIAKADTI | 570 | 11~L~AK | 599 | −1.98 | −0.40 | −2.69 | −1.64 | −2.10 | −1.72 | −1.48 | −0.93 | −1.15 | −1.42 | −0.21 | −0.89 |
| KVNIIPLIAKADTIS | 571 | IP~I~KA | 600 | −2.65 | 0.98 | −2.97 | −1.81 | −2.72 | −2.32 | −2.01 | −0.63 | −0.32 | −3.76 | 0.36 | 1.25 |
| VNIIPLIAKADTISK | 572 | PL~A~AD | 601 | −2.10 | −0.17 | −1.47 | −2.13 | −1.24 | −1.60 | −1.84 | −2.56 | −1.46 | −1.39 | −0.06 | −1.47 |
| NIIPLIAKADTISKN | 573 | LI~K~DT | 602 | −3.10 | −0.50 | −1.54 | −1.20 | −1.92 | −1.33 | −2.14 | −0.97 | −1.45 | −1.48 | 0.41 | −1.89 |
| IIPLIAKADTISKND | 574 | IA~A~TI | 603 | −1.63 | −0.67 | −0.65 | 0.07 | −1.26 | −1.13 | −1.08 | −0.73 | −1.55 | 0.60 | 0.77 | −0.70 |

#Footnotes as for Table 1

TABLE 12
Mutations commonly reported in EGFR include A289V, A289D, A289T and G598V or G598D in
glioblastomas and L585R in lunch cancer. Table 12 shows the T cell exposed
motifs which are tumor specific and associated with these mutations and those
arising from the common vIII variant.

| Mutant | | | | | | |
|---|---|---|---|---|---|---|
| EGFR | TCEM I | SEQ ID NO.: | TCEM IIa | SEQ ID NO.: | TCEMIIb | SEQ ID NO.: |
| A289D | | | GK~S~GD | 639 | P~~K~S~GD | 674 |
| A289D | | | KY~F~DT | 640 | E~~Y~F~DT | 675 |
| A289D | ~~~YSFGD~ | 604 | SF~D~CV | 641 | K~~F~D~CV | 676 |
| A289D | ~~~SFGDT~ | 605 | | | | |
| A289D | ~~~FGDTC~ | 606 | GD~C~KK | 642 | S~~D~C~KK | 677 |
| A289L | ~~~GDTCV~ | 607 | DT~V~KC | 643 | | |
| A289D | ~~~DTCVK~ | 608 | | | | |
| A289D | | | | | D~~V~K~PR | 678 |
| A289V | | | GK~S~GV | 644 | P~~K~S~GV | 679 |
| A289V | | | KY~F~VT | 645 | E~~Y~F~VT | 680 |
| A289V | ~~~YSFGV~ | 609 | SF~V~CV | 646 | K~~F~V~CV | 681 |
| A289V | ~~~SFGVT~ | 610 | | | | |
| A289V | ~~~FGVTC~ | 611 | GV~C~KK | 647 | S~~V~C~KK | 682 |
| A289V | ~~~GVTCV~ | 612 | VT~V~KC | 648 | | |
| A289V | ~~~VTCVK~ | 613 | | | | |
| A289V | | | | | V~~V~K~PR | 683 |
| G289T | | | GK~S~GT | 649 | P~~K~S~GT | 684 |
| G289T | | | KY~F~TT | 650 | E~~Y~F~TT | 685 |
| G289T | ~~~YSFGT~ | 614 | SF~T~CV | 651 | K~~F~T~CV | 686 |
| G289T | ~~~SFGTT~ | 615 | | | | |
| G289T | ~~~FGTTC~ | 616 | GT~C~KK | 652 | S~~T~C~KK | 687 |
| G289T | ~~~GTTCV~ | 617 | TT~V~KC | 653 | | |
| G289T | ~~~TTCVK~ | 618 | | | | |
| G289T | | | | | T~~V~K~PR | 688 |
| G598D | | | VK~C~AD | 654 | H~~K~C~AD | 689 |
| G598D | | | KT~P~DV | 655 | C~~T~P~DV | 690 |
| G598D | ~~~TCPAD~ | 619 | CP~D~MG | 656 | K~~P~D~MG | 691 |
| G598D | ~~~CPADV~ | 620 | | | | |
| G598D | ~~~PADVM~ | 621 | AD~M~EN | 657 | C~~D~M~EN | 692 |
| G598D | ~~~ADVMG~ | 622 | DV~G~NN | 658 | | |
| G598D | ~~~DVMGE~ | 623 | | | | |
| G598D | | | | | D~~G~N~TL | 693 |
| G598V | | | VK~C~AV | 659 | H~~K~C~AV | 694 |
| G598V | | | KT~P~VV | 660 | C~~T~P~VV | 695 |
| G598V | ~~~TCPAV~ | 624 | CP~V~MG | 661 | K~~P~V~MG | 696 |
| G598V | ~~~CPAVV~ | 625 | | | | |
| G598V | ~~~PAVVM~ | 626 | AV~M~EN | 662 | C~~V~M~EN | 697 |
| G598V | ~~~AVVMG~ | 627 | VV~G~NN | 663 | | |
| G598V | ~~~VVMGE~ | 628 | | | | |
| G598V | | | | | V~~G~N~TL | 698 |
| L858R | | | KI~D~GR | 664 | H~~~~D~GR | 699 |
| L858R | | | IT~F~RA | 665 | V~~T~F~RA | 700 |
| L858R | ~~~TDFGR~ | 629 | DF~R~KL | 666 | I~~F~R~KL | 701 |
| L858R | ~~~DFGRA~ | 630 | | | | |
| L858R | ~~~FGRAK~ | 631 | GR~K~LG | 667 | D~~R~K~LG | 702 |
| L858R | ~~~GRAKL~ | 632 | RA~L~GA | 668 | | |
| L858R | ~~~RAKLL~ | 633 | | | | |
| L858R | | | | | R~~L~G~EE | 703 |
| variant | TCEMI | | TCEMIla | | TCEMIlb | |
| vIII | | | AL~E~KG | 669 | S~~~E~KG | 704 |
| vIII | | | LE~K~GN | 670 | R~~E~K~GN | 705 |
| vIII | ~~~EEKKG~ | 634 | EK~G~YV | 671 | L~~K~G~YV | 706 |
| vIII | ~~~EKKGN~ | 635 | | | | |
| vIII | ~~~KKGNY~ | 636 | KG~Y~VT | 672 | E~~G~Y~VT | 707 |
| vIII | ~~~KGNYV~ | 637 | GN~V~TD | 673 | | |
| vIII | ~~~GNYVV~ | 638 | | | | |
| vIII | | | | | G~~V~T~HG | 708 |

TABLE 13
Peptides with customized groove exposed motifs to optimize
binding for representative alleles

| | | | | | Predicted | | | Predicted |
|---|---|---|---|---|---|---|---|---|
| | SEQ | | | SEQ | binding of | | SEQ | binding of |
| TCEM | ID | | Originating | ID | originating | proposed | ID | proposed |
| core | NO: | Allele | peptide | NO: | peptide | peptide | NO: | peptide |
| EEKKG | 709 | A_0301 | RALEEKKGN | 713 | −1.31 | RPAEEKKGK | 717 | −2.04 |
| EEKKG | 709 | A_0301 | RALEEKKGN | 713 | −1.31 | RFAEEKKGK | 718 | −2.02 |
| EEKKG | 709 | B_3501 | RALEEKKGN | 713 | −0.53 | FDSEEKKGW | 719 | −2.00 |
| EEKKG | 709 | B_3501 | RALEEKKGN | 713 | −0.53 | WACEEKKGG | 720 | −2.00 |
| EKKGN | 710 | A_0301 | ALEEKKGNY | 714 | −1.05 | FTPEKKGNK | 721 | −2.01 |
| EKKGN | 710 | A_0301 | ALEEKKGNY | 714 | −1.05 | KSDEKKGNK | 722 | −2.00 |
| EKKGN | 710 | A_0301 | ALEEKKGNY | 714 | −1.05 | RLAEKKGNY | 723 | −1.99 |
| EKKGN | 710 | A_0301 | ALEEKKGNY | 714 | −1.05 | NVWEKKGNR | 724 | −1.93 |
| EKKGN | 710 | B_3501 | ALEEKKGNY | 714 | −0.96 | WSEEKKGNL | 725 | −2.05 |
| EKKGN | 710 | B_3501 | ALEEKKGNY | 714 | −0.96 | FVKEKKGNY | 726 | −2.05 |
| EKKGN | 710 | C_0401 | ALEEKKGNY | 714 | −0.10 | SFEEKKGNL | 727 | −2.03 |
| EKKGN | 710 | C_0401 | ALEEKKGNY | 714 | −0.10 | PFEEKKGNA | 728 | −2.02 |
| KKGNY | 711 | B_4402 | LEEKKGNYV | 715 | −1.39 | RCEKKGNYS | 729 | −2.03 |
| KKGNY | 711 | B_4402 | LEEKKGNYV | 715 | −1.39 | TEPKKGNYV | 730 | −2.02 |
| KGNYV | 712 | B_4402 | EEKKGNYVV | 716 | −0.99 | YCSKGNYVA | 731 | −2.05 |
| KGNYV | 712 | B_4402 | EEKKGNYVV | 716 | −0.99 | AESKGNYVK | 732 | −2.04 |
| KGNYV | 712 | C_0401 | EEKKGNYVV | 716 | −0.58 | LEPKGNYVS | 733 | −2.01 |
| KGNYV | 712 | C_0401 | EEKKGNYVV | 716 | 0.58 | WKYKGNYVG | 734 | −2.00 |

1. Vogelstein B, Papadopoulos N, Velculescu V E, Zhou S, Diaz L A, Jr., Kinzler K W. Cancer genome landscapes. Science. 2013; 339(6127):1546-58. Epub 2013/03/30. doi: 10.1126/science.1235122. PubMed PMID: 23539594; PubMed Central PMCID: PMCPMC3749880.
2. Deshpande V, Luebeck J, Nguyen N D, Bakhtiari M, Turner K M, Schwab R, et al. Exploring the landscape of focal amplifications in cancer using AmpliconArchitect. Nature communications. 2019; 10(1):392. Epub 2019/01/25. doi: 10.1038/s41467-018-08200-y. PubMed PMID: 30674876; PubMed Central PMCID: PMCPMC6344493.
3. Turner K M, Deshpande V, Beyter D, Koga T, Rusert J, Lee C, et al. Extrachromosomal oncogene amplification drives tumour evolution and genetic heterogeneity. Nature. 2017; 543(7643):122-5. Epub 2017/02/09. doi: 10.1038/nature21356. PubMed PMID: 28178237; PubMed Central PMCID: PMCPMC5334176.
4. Lefranc M P, Giudicelli V, Ginestoux C, Jabado-Michaloud J, Folch G, Bellahcene F, et al. IMGT, the international ImMunoGeneTics information system. Nucleic acids research. 2009; 37 (Database issue):D1006-12. Epub 2008/11/04. doi: 10.1093/nar/gkn838. PubMed PMID: 18978023; PubMed Central PMCID: PMC2686541.
5. Vogt N, Lefevre S H, Apiou F, Dutrillaux A M, Cor A, Leuraud P, et al. Molecular structure of double-minute chromosomes bearing amplified copies of the epidermal growth factor receptor gene in gliomas. Proc Natl Acad Sci USA. 2004; 101(31):11368-73. Epub 2004/07/23. doi: 10.1073/pnas.0402979101. PubMed PMID: 15269346; PubMed Central PMCID: PMCPMC509208.
6. Vogt N, Gibaud A, Lemoine F, de la Grange P, Debatisse M, Malfoy B. Amplicon rearrangements during the extrachromosomal and intrachromosomal amplification process in a glioma. Nucleic acids research. 2014; 42(21):13194-205. Epub 2014/11/08. doi: 10.1093/nar/gku1101. PubMed PMID: 25378339; PubMed Central PMCID: PMCPMC4245956.
7. Gu X, Yu J, Chai P, Ge S, Fan X. Novel insights into extrachromosomal DNA: redefining the onco-drivers of tumor progression. J Exp Clin Cancer Res. 2020; 39(1):215. Epub 2020/10/14. doi: 10.1186/s13046-020-01726-4. PubMed PMID: 33046109; PubMed Central PMCID: PMCPMC7552444.
8. Zhou Y H, Chen Y, Hu Y, Yu L, Tran K, Giedzinski E, et al. The role of EGFR double minutes in modulating the response of malignant gliomas to radiotherapy. Oncotarget. 2017; 8(46):80853-68. Epub 2017/11/09. doi: 10.18632/oncotarget.20714. PubMed PMID: 29113349; PubMed Central PMCID: PMCPMC5655244.
9. Koche R P, Rodriguez-Fos E, Helmsauer K, Burkert M, MacArthur I C, Maag J, et al. Extrachromosomal circular DNA drives oncogenic genome remodeling in neuroblastoma. Nat Genet. 2020; 52(1):29-34. Epub 2019/12/18. doi: 10.1038/s41588-019-0547-z. PubMed PMID: 31844324; PubMed Central PMCID: PMCPMC7008131.
10. Nikolaev S, Santoni F, Garieri M, Makrythanasis P, Falconnet E, Guipponi M, et al. Extrachromosomal driver mutations in glioblastoma and low-grade glioma. Nature communications. 2014; 5:5690. Epub 2014/12/05. doi: 10.1038/ncomms6690. PubMed PMID: 25471132; PubMed Central PMCID: PMCPMC4338529.
11. Morales C, Garcia M J, Ribas M, Miro R, Munoz M, Caldas C, et al. Dihydrofolate reductase amplification and sensitization to methotrexate of methotrexate-resistant colon cancer cells. Mol Cancer Ther. 2009; 8(2):424-32. Epub 2009/02/05. doi: 10.1158/1535-7163.MCT-08-0759. PubMed PMID: 19190117.
12. Vicario R, Peg V, Morancho B, Zacarias-Fluck M, Zhang J, Martinez-Barriocanal A, et al. Patterns of HER2 Gene Amplification and Response to Anti-HER2 Therapies. PloS one. 2015; 10 (6):e0129876. Epub 2015/06/16. doi: 10.1371/journal.pone.0129876. PubMed PMID: 26075403; PubMed Central PMCID: PMCPMC4467984.
13. Jin Y, Liu Z, Cao W, Ma X, Fan Y, Yu Y, et al. Novel functional MAR elements of double minute chromosomes in human ovarian cells capable of enhancing gene expression. PloS one. 2012; 7 (2):e30419. Epub 2012/02/10. doi: 10.1371/journal.pone.0030419. PubMed PMID: 22319568; PubMed Central PMCID: PMCPMC3272018.
14. VanDevanter D R, Piaskowski V D, Casper J T, Douglass E C, Von Hoff D D. Ability of circular extrachromosomal DNA molecules to carry amplified MYCN proto-oncogenes in human neuroblastomas in vivo. Journal of the National Cancer Institute. 1990; 82(23):1815-21. Epub 1990/12/05. doi: 10.1093/jnci/82.23.1815. PubMed PMID: 2250296.
15. An Z, Aksoy O, Zheng T, Fan Q W, Weiss W A. Epidermal growth factor receptor and EGFRvIII in glioblastoma: signaling pathways and targeted therapies. Oncogene. 2018; 37(12):1561-75. Epub 2018/01/13. doi: 10.1038/s41388-017-0045-7. PubMed PMID: 29321659; PubMed Central PMCID: PMCPMC5860944.
16. Daubon T, Hemadou, A., Romero-Garmendia, I., Saleh, M. Glioblastoma Immune Landscape and the Potential of New Immunotherapies. Frontiers in immunology. 2020; 11:Article 585616. doi: doi: 10.3389/fimmu.2020.585616.
17. Lawson K A, Sousa C M, Zhang X, Kim E, Akthar R, Caumanns J J, et al. Functional genomic landscape of cancer-intrinsic evasion of killing by T cells. Nature. 2020; 586(7827):120-6. Epub 2020/09/25. doi: 10.1038/s41586-020-2746-2. PubMed PMID: 32968282.
18. Hobbs J, Nikiforova M N, Fardo D W, Bortoluzzi S, Cieply K, Hamilton R L, et al. Paradoxical relationship between the degree of EGFR amplification and outcome in glioblastomas. Am J Surg Pathol. 2012; 36(8):1186-93. Epub 2012/04/05. doi: 10.1097/PAS.0b013e3182518e12. PubMed PMID: 22472960; PubMed Central PMCID: PMCPMC3393818.
19. Francis J M, Zhang C Z, Maire C L, Jung J, Manzo V E, Adalsteinsson V A, et al. EGFR variant heterogeneity in glioblastoma resolved through single-nucleus sequencing. Cancer Discov. 2014; 4(8):956-71. Epub 2014/06/05. doi: 10.1158/2159-8290.CD-13-0879. PubMed PMID: 24893890; PubMed Central PMCID: PMCPMC4125473.
20. Lu Z, Zhou L, Killela P, Rasheed A B, Di C, Poe W E, et al. Glioblastoma proto-oncogene SEC61gamma is required for tumor cell survival and response to endoplasmic reticulum stress. Cancer Res. 2009; 69(23):9105-11. Epub 2009/11/19. doi: 10.1158/0008-5472.CAN-09-2775. PubMed PMID: 19920201; PubMed Central PMCID: PMCPMC2789175.
21. Liu B, Liu J, Liao Y, Jin C, Zhang Z, Zhao J, et al. Identification of SEC61G as a Novel Prognostic Marker for Predicting Survival and Response to Therapies in Patients with Glioblastoma. Med Sci Monit. 2019; 25:3624-35. Epub 2019/05/17. doi: 10.12659/MSM.916648. PubMed PMID: 31094363; PubMed Central PMCID: PMCPMC6536036.
22. Richters M M, Xia H, Campbell K M, Gillanders W E, Griffith O L, Griffith M. Best practices for bioinformatic characterization of neoantigens for clinical utility. Genome Med. 2019; 11(1):56. Epub 2019/08/30. doi: 10.1186/s13073-019-0666-2. PubMed PMID: 31462330; PubMed Central PMCID: PMCPMC6714459.
23. Szolek A, Schubert B, Mohr C, Sturm M, Feldhahn M, Kohlbacher O. OptiType: precision HLA typing from next-generation sequencing data. Bioinformatics. 2014; 30(23):3310-6. Epub 2014/08/22. doi: 10.1093/bioinformatics/btu548. PubMed PMID: 25143287; PubMed Central PMCID: PMCPMC4441069.
24. Osborne A R, Rapoport T A, van den Berg B. Protein translocation by the Sec61/SecY channel. Annu Rev Cell Dev Biol. 2005; 21:529-50. Epub 2005/10/11. doi: 10.1146/annurev.cellbio.21.012704.133214. PubMed PMID: 16212506.
25. Liao H J, Carpenter G. Role of the Sec61 translocon in EGF receptor trafficking to the nucleus and gene expression. Mol Biol Cell. 2007; 18(3):1064-72. Epub 2007/01/12. doi: 10.1091/mbc.e06-09-0802. PubMed PMID: 17215517; PubMed Central PMCID: PMCPMC1805100.
26. Servidei T, Meco D, Muto V, Bruselles A, Ciolfi A, Trivieri N, et al. Novel SEC61G-EGFR Fusion Gene in Pediatric Ependymomas Discovered by Clonal Expansion of Stem Cells in Absence of Exogenous Mitogens. Cancer Res. 2017; 77(21):5860-72. Epub 2017/11/03. doi: 10.1158/0008-5472.CAN-17-0790. PubMed PMID: 29092923.
27. Neidert M C, Schoor O, Trautwein C, Trautwein N, Christ L, Melms A, et al. Natural HLA class I ligands from glioblastoma: extending the options for immunotherapy. J Neurooncol. 2013; 111(3):285-94. Epub 2012/12/25. doi: 10.1007/s11060-012-1028-8. PubMed PMID: 23263746.
28. Park S, James C D. ECop (EGFR-coamplified and overexpressed protein), a novel protein, regulates NF-kappaB transcriptional activity and associated apoptotic response in an IkappaBalpha-dependent manner. Oncogene. 2005; 24(15):2495-502. Epub 2005/03/01. doi: 10.1038/sj.onc.1208496. PubMed PMID: 15735698.
29. Baras A, Moskaluk C A. Intracellular localization of GASP/ECOP/VOPP1. J Mol Histol. 2010; 41 (2-3):153-64. Epub 2010/06/24. doi: 10.1007/s10735-010-9272-8. PubMed PMID: 20571887.
30. Eley G D, Reiter J L, Pandita A, Park S, Jenkins R B, Maihle N J, et al. A chromosomal region 7p11.2 transcript map: its development and application to the study of EGFR amplicons in glioblastoma. Neuro Oncol. 2002; 4(2):86-94. Epub 2002/03/28. doi: 10.1093/neuonc/4.2.86. PubMed PMID: 11916499; PubMed Central PMCID: PMCPMC1920657.
31. Baras A S, Solomon A, Davidson R, Moskaluk C A. Loss of VOPP1 overexpression in squamous carcinoma cells induces apoptosis through oxidative cellular injury. Lab Invest. 2011; 91(8):1170-80. Epub 2011/04/27. doi: 10.1038/labinvest.2011.70. PubMed PMID: 21519330.
32. Park S, James C D. Lanthionine synthetase components C-like 2 increases cellular sensitivity to adriamycin by decreasing the expression of P-glycoprotein through a transcription-mediated mechanism. Cancer Res. 2003; 63(3):723-7. Epub 2003/02/05. PubMed PMID: 12566319.
33. Rozenkrantz L, Gan-Or Z, Gana-Weisz M, Mirelman A, Giladi N, Bar-Shira A, et al. SEPT14 Is Associated with a Reduced Risk for Parkinson's Disease and Expressed in Human Brain. J Mol Neurosci. 2016; 59(3):343-50. Epub 2016/04/27. doi: 10.1007/s12031-016-0738-3. PubMed PMID: 27115672.
34. Zhu Y C, Wang W X, Li X L, Xu C W, Chen G, Zhuang W, et al. Identification of a Novel Icotinib-Sensitive EGFR-SEPTIN14 Fusion Variant in Lung Adenocarcinoma by Next-Generation Sequencing. J Thorac Oncol. 2019; 14 (8):e181-e3. Epub 2019/07/28. doi: 10.1016/j.jtho.2019.03.031. PubMed PMID: 31345345.

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

1. A method for treating cancer in a subject, comprising:
designing a group of one or more T-cell stimulating peptides, or nucleic acids encoding T cell stimulating peptides, which have a desired predicted binding affinity for the MHC alleles of the subject, comprising the following steps:
obtaining a biopsy of the subject's tumor;
obtaining sequences for nucleic acids and proteins in the biopsy;
comparing the copy number differential of genes encoding each protein between tumor and normal tissue;
identifying proteins from the biopsy comprising an oncogene which is upregulated;
identifying bystander proteins of the proteins that are transcribed;
determining T cell exposed motifs in each of the bystander proteins;
determining the predicted binding affinity to the subject's MHC alleles of peptides which comprises each of the T cell exposed motifs, or a subset thereof;
selecting a group of one or more the peptides which have a desired predicted binding affinity for one or more of the subject's MHC alleles;
synthesizing the group of one or more selected peptides, or nucleic acids encoding the selected peptides from the bystander proteins; and
administering the selected peptides or nucleic acids to the subject.

2. The method of claim 1, further comprising generating one or more alternative peptides not present in the tumor biopsy, wherein each alternative peptide comprises a T cell exposed motif identified in the bystander proteins, and in which the amino acids not within the T cell exposed motif are substituted to change the predicted binding affinity to the MHC alleles.

3. The method of claim 1, wherein the oncogene is mutated in the tumor biopsy relative to the normal tissue.

4. The method of claim 1, wherein the genes encoding the bystander proteins are present in increased copy number in the tumor biopsy.

5. The method of claim 1, wherein the copy number in the tumor biopsy of the oncogene is increased by more than five-fold over that in the normal tissue.

6. The method of claim 1, wherein the copy number in the tumor biopsy of the oncogene is increased by more than ten-fold over that in the normal tissue.

7. The method of claim 1, wherein the MHC allele is an MHC I allele.

8. The method of claim 1, wherein the MHC allele is an MHC II allele.

9. The method of claim 1, wherein the selected peptides are 9 or 10 amino acids long.

10. The method of claim 1, wherein the selected peptides are 13 to 20 amino acids long.

11. The method of claim 1, wherein the selected peptides are from 8 to 30 amino acids long.

12. The method of claim 2, wherein the predicted binding MHC affinity is to an MHC I allele carried by the subject.

13. The method of claim 2, wherein the predicted binding MHC affinity is to an MHC II allele carried by the subject.

14. The method of claim 1, wherein the desired predicted binding affinity of each selected peptide is less than 20 nanomolar.

15. The method of claim 1, wherein the desired predicted binding affinity of each selected peptide is less than 50 nanomolar.

16. The method of claim 1, wherein the desired predicted binding affinity of each selected peptide is less than 100 nanomolar.

17. The method of claim 1, wherein the desired predicted binding affinity of each selected peptide is less than 500 nanomolar.

18. The method of claim 1, wherein the cancer with which the subject is afflicted with is selected from the group consisting of lung cancer, breast cancer, brain cancer, liver cancer, prostate cancer, pancreatic cancer, renal cancer, ovarian or uterine cancer, gastrointestinal tract cancer and a hematologic cancer.

19. The method of claim 18, wherein the brain cancer is selected from the group consisting of glioma, glioblastoma, meningioma, astrocytoma, medulloblastoma, schwannoma and a metastasis from an extracranial site.

20. The method of claim 1, wherein the oncogene is selected from the group consisting of EGFR, PDGFA, ERRB2, MDM2, MYC, MYON, and CDK4 and combinations thereof.

21-57. (canceled)
